(19) United States
(12) Patent Application Publication
(10) Pub. No.: US 2009/0062253 A1
(43) Pub. Date: Mar. 5, 2009

(54) HETEROCYCLODIAZEPINE CANNABINOID RECEPTOR MODULATORS FOR TREATMENT OF DISEASE

(75) Inventors: Timothy C. Gahman, Encinitas, CA (US); Cunxiang Zhao, San Diego, CA (US); Hengyuan Lang, San Diego, CA (US); Mark Eben Massari, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 12/200,554

(22) Filed: Aug. 28, 2008

Related U.S. Application Data
(60) Provisional application No. 60/969,174, filed on Aug. 31, 2007.

(51) Int. Cl. A61K 31/551; C07D 471/04; C07D 487/04; A61K 31/55; A61P 25/00; A61K 31/553; C07D 498/04; A61K 31/4985

(52) U.S. Cl. 514/211.1; 544/349; 540/567; 540/568; 540/593; 540/552; 514/249; 514/221; 514/214.02

(57) ABSTRACT

The present invention relates to compounds and methods useful as modulators of CB2 for the treatment or prevention of disease states including, but not limited to pain, autoimmune disease, malabsorption syndrome, pulmonary disease, osteoporosis, muscle spasm in cancer, neuromuscular disorder, and atherosclerosis progression.

This application claims the benefit of priority of U.S. provisional application No. 60/969,174, filed Aug. 31, 2007, the disclosure of which is hereby incorporated by reference as if written herein in its entirety.

Disclosed herein are new heterocyclic compounds and compositions and their application as pharmaceuticals for the treatment of disease. Methods of modulation of CB2 activity in a human or animal subject are also provided for the treatment diseases mediated by CB2.

Preparations of Cannabis sativa have been used for medicinal and recreational purposes for at least 4,000 years. Recently, cannabinoids have been the subject of renewed interest for their potential therapeutic applications (Mechoulam, R. in “Cannabinoids as Therapeutic Agents” CRC Press, Boca Raton, Fla., 1-19, 1986). The native active constituent, Delta 9-tetrahydrocannabinol (Δ9-THC), is prescribed today, under the generic name Dronabinol, as an anti-emetic and for enhancement of appetite, mainly in AIDS patients. However, separation between the clinically undesirable psychotropic effects and the therapeutically desirable effects, such as vascular hypotension and immunomodulation, has only recently been accomplished. The discovery and molecular cloning of the cannabinoid receptors has helped to elucidate the diverse cannabinoid effects.

Cannabinoids exert their effects by binding to specific receptors located in the cell membrane. Two types of high-affinity cannabinoid receptors have been identified to date by molecular cloning: 1) CB1 receptors (Devane et al., 1988, Mol. Pharmacol., 34:605-613; Matsuda et al., 1990, Nature, 346:561-564; Shire et al., 1995, J. Biol. Chem., 270:3726-373 1; Ishac et al., 1996, Br. J. Pharmacol., 118:2023-2028), and 2) CB2 receptors (Munro et al., 1993, Nature, 365:61-65). CB1 and CB2, which share 44% identity at the amino acid level, are members of the G protein-coupled receptor (GPCRs) family. Both CB1 and CB2 couple to the inhibitory G-protein alpha-subunit Gi. Receptor activation thus leads to inhibition of adenylate cyclase as well as to activation of mitogen activated protein kinase (MAPK) (Parolaro, D., Life Sci. 65: 637-44, 1999). CB1 receptors can also modulate ion channels, inhibiting N-, and P/R-type calcium channels, stimulating inwardly rectifying K channels and enhancing the activation of the A-type K channel.

CB1 receptors are primarily, but not exclusively, expressed in the CNS and are believed to mediate the CNS effects of endogenous (e.g., anandamide, 2-arachidonoylglycerol [2-AG]) and exogenously applied cannabinoids. Peripheral areas of expression include, but are not restricted to, the pituitary gland, immune cells, reproductive tissues, gastrointestinal tissues, superior cervical ganglion, heart, lung, urinary bladder, and adrenal gland. CB1 receptors are also located on central and peripheral nerve terminals and, when activated, seem to suppress the neuronal release of a number of excitatory and inhibitory transmitters including acetyicholine, noradrenaline, dopamine, 5-hydroxytryptamine, γ-aminobutyric acid, glutamate and aspartate (Pertwee, 1997, Pharmacol. Ther., 129:74; Ong & Macide, 1999, Neuroscience, 92:1177; Pertwee, 2001, Progr. Neurobiol., 63:569). CB2 receptor expression was originally thought to be restricted to the periphery, mainly in lymphoid organs and cells of the immune system, including spleen, thymus, tonsils, bone marrow, pancreas and mast cells with particularly high levels in B-cells and natural killer cells (Galiègue et al., 1995, Bur. J. Biochein, 54:232). However, recent studies demonstrate that CB2 is expressed in the brain stem, cortex, cerebellum and hippocampus (Onaivi et al., 2006, Ann. N.Y. Acad. Sci., 1074:514-36; Van Sickle et al. 2005, Science, 310 329-32). In addition, there are both electophysiological and in situ hybridization data that demonstrate expression of CB2 receptors in the dorsal root ganglion and primary sensory afferent fibers in the spinal cord (Elmes et al., 2004, Eur. J. Neurosci. 20: 2311-20; Wotherspoon et al., 2005, Neuroscience 135: 235-45; Zhang et al., 2003, Eur. J. Neurosci. 17: 2750-54).

The location of CB2 receptors on the surface of immune cells suggests a role for these receptors in immunomodulation and inflammation. Endogenous cannabinoids have been shown to act as immuno-modulators, generally exerting a negative action on the onset of a variety of parameters of the immune response (Parolaro et al., 2002, Prostaglandins Leukot. Essent. Fatty Acids, 66:319-32). Previous studies have shown that the CB2 receptor plays a very important role in the stimulation of growth of several, if not all, hematopoietic lineages (Valk et al., 1997, Blood, 90:1448-1457; Derocq, 2000, J. Biol. Chem, 275: 15621-15628). The role of the endocannabinoid system in immunosuppression is the focus of many studies (Berdyshev, E. V., Chem. Phys. Lipids 108: 169-90, 2000). Anandamide, Palmitoylethanolamide (PEA) and 2-AG were shown to down-regulate the immune response in a variety of experimental systems and function as anti-inflammatory and immunosuppressive agents.

Analysis of the CB2 knockout mouse has corroborated the evidence for the function of CB2 receptors in modulating the immune system. CB2 does not affect immune cell development and differentiation as determined by FACS analysis of cells from the spleen, lymph node and thymus from CB2 knockout mice, but rather mediates the suppressive effect of Δ9-THC. Therefore, compounds that selectively interact with CB2 receptors offer a unique pharmacotherapy for the treatment of immune and inflammatory disorders.

The psychotropic side-effects caused by Δ9-THC and other nonselective CB agonists are mediated by CB1 receptors. CB1 knockout mice have been shown to be unresponsive to cannabinoids in behavioral assays providing molecular evidence that the psychotropic effects, including sedation, hallucinations and delirium and anti-nociception are manifested through activation of the CB1 receptor, present primarily in the CNS. These CB1 receptor-mediated effects have limited the development and clinical utility of nonselective CB agonists.

Pain is the most common symptom of disease and the most frequent complaint with which patients present to physicians. Pain is commonly segmented by duration (acute vs. chronic), intensity (mild, moderate, and severe), and type (nociceptive vs. neuropathic).

Nociceptive pain is the most well known type of pain, and is caused by tissue injury detected by nociceptors at the site of injury. After the injury, the site becomes a source of ongoing pain and tenderness. This pain and tenderness are considered “acute” nociceptive pain. This pain and tenderness gradually diminish as healing progresses and disappear when healing is complete. Examples of acute nociceptive pain include surgical procedures (post-op pain) and bone fractures. Even though there may be no permanent nerve damage, “chronic” nociceptive pain results from some conditions when pain extends beyond six months. Examples of chronic nociceptive pain include osteoarthritis, rheumatoid arthritis, and musculoskeletal conditions (e.g., back pain), cancer pain, etc.

Neuropathic pain is defined as “pain initiated or caused by a primary lesion or dysfunction in the nervous system” by the International Association for the Study of Pain. Neuropathic pain is not associated with nociceptive stimulation, although the passage of nerve impulses that is ultimately perceived as pain by the brain is the same in both nociceptive and neuropathic pain. The term neuropathic pain encompasses a wide range of pain syndromes of diverse etiologies. The three most commonly diagnosed pain types of neuropathic nature are diabetic neuropathy, cancer neuropathy, and HIV pain. In addition, neuropathic pain is diagnosed in patients with a wide range of other disorders, including trigeminal neuralgia, post-herpetic neuralgia, traumatic neuralgia, phantom limb, as well as a number of other disorders of ill-defined or unknown origin.

Managing the spectrum of pain etiologies remains a major public health problem and both patients and clinicians are seeking improved strategies to effectively manage pain. No currently available therapies or drugs effectively treat all types of nociceptive and neuropathic pain states. The compounds of the present invention are novel CB2 receptor modulators that have utility in treating pain, including nociceptive and neuropathic pain.

Numerous studies have demonstrated that CB2-selective modulators are analgesic in preclinical models of nociceptive and neuropathic pain without causing the adverse side-effects associated with CB1 receptor activation (Malan et al., 2003, Curr. Opin. Pharmacol. 3: 62-7; Ibrahim et al., 2003, Proc. Natl. Acad. Sci. USA 100: 10529-33; Hanus et al., 1999, Proc. Natl. Acad. Sci. USA 96: 14228-33; Elmes et al., 2004, Eur. J. Neurosci. 20: 2311-20; Fox and Bevan, 2005, Expert Opin. Invest. Drugs 14: 695-703). For example, the CB2 receptor-selective compound AM1241 has been shown to be active in several animal models of pain, including spinal nerve ligation, acute thermal pain, carrageenan-induced thermal hyperalgesia and intradermal capsaicin-evoked hyperalgesia (Quartilho et al., 2003, Anesthesiology 99: 955-60; Hohmann et al., 2004, J. Pharmacol. Exp. Ther.: 308, 446-53). The CB2 receptor-selective partial agonist GW405833 has also been shown to be efficacious in inflammatory, neuropathic, and surgical models of pain (Valenzano et al., 2005, Neuropharmacology 48:658-72). A recent study revealed that oral delivery of Lactobacillus acidophilus induced the expression of CB2 receptors in the intestinal epithelium suggesting that CB2 receptor modulators may be useful for the treatment of abdominal pain associated with gastrointestinal diseases such as irritable bowel syndrome (Rousseaux et al., 2007, Nat. Med. 13: 35-37). Therefore, compounds that selectively target CB2 receptors represent an attractive approach for the development of novel analgesics.

Due to the restricted expression of the CB2 receptor in subsets of immune cells and neurons, selective CB2 ligands have therapeutic value (Pertwee, R. G., Curr. Med. Chem. 6: 63 5-64, 1999). Of particular interest are those compounds with high affinity and high specificity for the CB2 receptor. These compounds could afford the benefits of CB2 agonism while avoiding the adverse side effects seen in compounds with affinity for the CB1 receptor. Such compounds could be effective in the treatment of pain as well as autoimmune diseases including but not limited to multiple sclerosis, rheumatoid arthritis, systemic lupus erythematosus, myasthenia gravis, diabetes mellitus type I, inflammatory bowel disease or irritable bowel syndrome, psoriasis and other immune related disorders including but not limited to tissue rejection in organ transplants, malabsorption syndromes such as celiac disease, pulmonary diseases such as asthma and Sjogren's syndrome. The discovery of cannabinoid receptors and the more recent identification of endocannabinoids, endogenous ligands capable of activating the CB receptors, has led to the understanding of the multiplicity of effects exerted by cannabinoids and related compounds. On top of a general neuroprotective effect of certain cannabinoid agonists more specific applications can be found. Thus, for example, evidence for the tonic control of spasticity by the endocannabinoid system suggests that cannabinoid agonists may help in the treatment of muscle spasm and tremor in multiple sclerosis (Baker D. et al., FASEB 3. 15: 300-2, 2001), in addition to the possible moderation of the disease by immuno-modulation through an action on CB2 receptors expressed by immune cells. Cannabinoid agonists may also prove to be of help in the treatment muscle spasm in cancer and REV/AIDS (Hall W. D., Degenhardt L. J. & Currow D., Med. J. Aust. 175: 39-40, 2001) and of neuromuscular disorders.

Recently, studies have demonstrated a potential therapeutic benefit for CB2-selective agonists for the treatment of osteoporosis. CB2 is expressed in osteoblasts, osteocytes and osteoclasts. The CB2-selective agonist HU-308 mitigates ovariectomy-induced bone loss in mice (Ofek et al., 2006, Proc. Natl. Acad. Sci. USA 103 696-701). Consistent with these findings, CB2 knockout mice were shown to have reduced bone mass.

CB2 agonists are also of potential benefit for the treatment of atherosclerosis. Low dose treatment of apoE knockout mice with Δ9-THC has been shown to reduce atherosclerosis progression. Furthermore, these effects are abrogated by treatment with a CB2-selective antagonist (Steffens et al., 2005, Nature 434 782-86).

Liver fibrosis is driven by chronic liver injury and ultimately leads to the development of cirrhosis. Recent studies have shown that CB2 modulators may be of benefit for the treatment of liver diseases such as liver fibrosis, ischemia-reperfusion injury, hepatic encephalopathy and non-alcoholic fatty liver disease (NAFLD). CB2 receptors are expressed in hepatocytes derived from individuals diagnosed with NAFLD but not from normal liver samples (Mendez-Sanchez et al., 2007, Liver Int. 27(2) 215-219). Expression of CB2 has also been shown to be highly upregulated in myofibroblasts isolated from cirrhotic human livers (Julien et al. 2006, Gastroenterology 128 742-755). In a mouse model of liver fibrosis, CB2 knockout animals displayed a significantly enhanced fibrotic phenotype as compared to wild type controls (Lotersztajn et al. 2008, Br. J. Pharmacol. 153(2):286-89 ). Interestingly, treatment of liver myofibroblasts with a CB2 agonist results in inhibition of cell growth and triggers apoptosis (Julien et al. 2006, Gastroenterology 128 742-755). Thus, activation of CB2 may limit fibrosis by interfering with the growth of liver fibrogenic cells. Taken together, these data suggest that CB2-selective agonists hold promise as therapeutics for a range of liver diseases.

Several synthetic compounds have been shown to bind to the CB2 receptor with a higher affinity than to the CB1 receptor (Pertwee, R. G., Expert Opin. Investig. Drugs 9: 1553- 71, 2000). Cannabinoid receptor agonists comprise four main groups of compounds. The classic cannabinoids maintain the dibenzopyran ring system of THC while the non-classical cannabinoids include bicydic or tricyclic analogs lacking the pyran ring. The aminoalkylindoles and analogs make up the third family and the endocannabinoids including anandamide and other fatty acid derivatives comprise the fourth family. For instance, L5 759656 is a classical cannabinoid analog and HTJ-308 is a bicyclic analog. Both have CB2/CB1 binding affinity ratios of 300-400 and both have been shown to behave as potent and specific CB2 agonists in functional assays (Hand, L. et al., Proc. Natl. Acad. Sci. USA 96: 14228-33, 1999; Ross, R. A. et al., Br. J. Pharmacol. 126: 665-72, 1999).

Compounds disclosed herein are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds are also useful in the treatment of pain associated with acute herpes zoster (shingles), postherpetic neuralgia (PHN), and associated pain syndromes such as ocular pain. The compounds are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, bunionectomy, pain resulting from cancer, muscular pain, mastalgia, pain resulting from dermal injuries, lower back pain, headaches of various etiologies, including migraine, and the like. The compounds are also useful for the treatment of pain-related disorders such as tactile allodynia and hyperalgesia. The compounds are also useful for the treatment of glaucoma. The pain may be somatogenic (either nociceptive or neuropathic), acute and/or chronic.

Novel compounds and pharmaceutical compositions, certain of which have been found to modulate CB2 have been discovered, together with methods of synthesizing and using the compounds including methods for the treatment of CB2-mediated diseases in a patient by administering the compounds.

In certain embodiments of the present invention, compounds have structural Formula I:

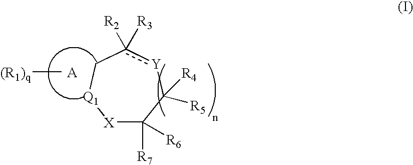

Or a salt, ester, or prodrug thereof, wherein:

A is a five- or six-membered monocyclic heterocycloalkyl or heteroaryl ring;

X is selected from the group consisting of CR₈R₉ and O;

Y is selected from the group consisting of NR₁₀ and CR₁₁R₁₂;

Q₁ is selected from the group consisting of N and CR₁₃;

n is an integer from 0 to 2;

q is an integer from 0 to 4;

each R₁ is independently selected from the group consisting of hydrogen, null, acyl, alkyl, alkenyl, alkynyl, alkoxy, amido, amino, aryl, aryloxy, carbamate, carboxy, cyano, cycloalkyl, halo, heteroalkyl, heteroaryl, heterocycloalkyl, hydroxyl, nitro, perhaloalkoxy, perhaloalkyl, and sulfonamide, any of which may be optionally substituted;

R₂ and R₃ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₄, R₅, R₆, R₇, R₈, and R₉ are each independently selected from the group consisting of hydrogen, null, alkyl, alkenyl, alkynyl, aryl, heteroaryl, cycloalkyl, and heterocycloalkyl, any of which may be optionally substituted; or R₆ and R₇ are taken together to form oxo (═O);

R₁₀ is selected from the group consisting of hydrogen, alkyl, alkenyl, alkynyl, aryl, arylalkyl, heteroaryl, cycloalkyl, heterocycloalkyl, —C(O)R₁₄, —C(O)NR₁₅R₁₆, and sulfonyl, any of which may be optionally substituted;

R₁₁ and R₁₂ are each independently selected from the group consisting of hydrogen, null, alkyl, alkenyl, alkynyl, aryl, arylalkyl, heteroaryl, cycloalkyl, heterocycloalkyl, —C(O)NR₁₅R₁₆, —NR₁₇C(O)NR₁₈R₁₉, —NR₂₀C(O)OR₂₁, and sulfonyl, any of which may be optionally substituted;

R₁₃, R₁₅, R₁₇, R₁₉ and R₂₀ are each independently selected from the group consisting of hydrogen, null, and lower alkyl; and

R₁₄, R₁₆, R₁₈ and R₂₁ are each independently selected from the group consisting of hydrogen, alkyl, alkenyl, alkynyl, aryl, heteroaryl, cycloalkyl, heterocycloalkyl, arylalkyl, heteroarylalkyl, and cycloalkylalkyl, any of which may be optionally substituted.

Compounds disclosed herein possess useful CB2 modulating activity, and may be used in the treatment or prophylaxis of a disease or condition in which CB2 plays an active role. Thus, in broad aspect, also provided herein are pharmaceutical compositions, comprising one or more compounds, disclosed herein together with a pharmaceutically acceptable carrier, as well as methods of making and using the compounds and compositions. In certain embodiments are provided methods for modulating CB2. In other embodiments are provided methods for treating a CB2-mediated disorder in a patient in need of such treatment comprising administering to said patient a therapeutically effective amount of a compound or composition according to the present invention. Also provided is the use of compounds disclosed herein for use in the manufacture of a medicament for the treatment of a disease or condition ameliorated by the modulation of CB2.

In certain embodiments, the compounds have structural Formula II:

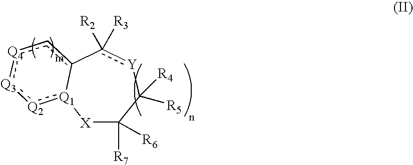

Or a salt, ester, or prodrug thereof, wherein:

X is selected from the group consisting of CR₈R₉ and O;

Y is selected from the group consisting of NR₁₀ and CR₁₁R₁₂;

Q₁ is selected from the group consisting of N and CR₁₃;

Q₂ is selected from the group consisting of N, NR₂₂, CR₂₃, and CR₂₃R₂₄;

Q₃ is selected from the group consisting of N, NR₂₅, CR₂₆, CR₂₆R₂₇, S, and O;

Q₄ is selected from the group consisting of N, NR₂₈, CR₂₉, CR₂₉R₃₀, S, and O;

m is an integer from 0 to 2;

n is an integer from 0 to 2;

m is an integer from 0 to 2;

R₂ and R₃ are independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₄, R₅, R₆, R₇, R₈, and R₉ are each independently selected from the group consisting of hydrogen, null, alkyl, alkenyl, alkynyl, aryl, heteroaryl, cycloalkyl, and heterocycloalkyl, any of which may be optionally substituted; or R₆ and R₇ are taken together to form oxo (═O);

R₁₀ is selected from the group consisting of hydrogen, alkyl, alkenyl, alkynyl, aryl, arylalkyl, heteroaryl, cycloalkyl, heterocycloalkyl, —C(O)R₁₄, —C(O)NR₁₅R₁₆, and sulfonyl, any of which may be optionally substituted;

R₁₁ and R₁₂ are each independently selected from the group consisting of hydrogen, null, alkyl, alkenyl, alkynyl, aryl, arylalkyl, heteroaryl, cycloalkyl, heterocycloalkyl, —C(O)NR₁₅R₁₆, —NR₁₇C(O)NR₁₈R₁₉, —NR₂₀C(O)OR₂₁, and sulfonyl, any of which may be optionally substituted;

R₁₃, R₁₅, R₁₇, R₁₉, R₂₀, R₂₂, R₂₅, and R₂₈ are each independently selected from the group consisting of hydrogen, null, and lower alkyl; and

R₁₄, R₁₆, R₁₈, R₂₁, R₂₃, R₂₄, R₂₆, R₂₇, R₂₉, and R₃₀ are each independent selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

In further embodiments provided herein,

Y is NR₁₀; and

n is 1.

In further embodiments provided herein,

R₂, R₄, R₅, R₆, and R₇ are hydrogen; and

R₁₀ is —C(O)NR₁₅R₁₆.

In further embodiments provided herein, R₁₅ is hydrogen.

In certain embodiments, the compounds have structural Formula III:

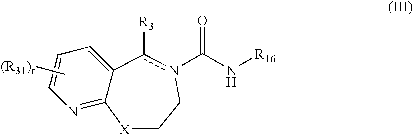

Or a salt, ester, or prodrug thereof, wherein:

X is selected from the group consisting of CR₈R₉ and O;

r is an integer from 0 to 3;

R₃ is selected from the group consisting of hydrogen alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₈ and R₉ are each independently selected from the group consisting of hydrogen, null, alkyl, alkenyl, alkynyl, aryl, heteroaryl, cycloalkyl, and heterocycloalkyl, any of which may be optionally substituted;

R₁₆ is selected from the group consisting of aryl, heteroaryl, and arylalkyl, any of which may be optionally substituted; and

each R₃₁ is independently selected from the group consisting of hydrogen, null, acyl, alkyl, alkenyl, alkynyl, alkoxy, amido, amino, aryl, aryloxy, carbamate, carboxy, cyano, cycloalkyl, halo, heteroalkyl, heteroaryl, heterocycloalkyl, hydroxyl, nitro, perhaloalkoxy, perhaloalkyl, and sulfonamide, any of which may be optionally substituted.

In further embodiments provided herein, R₃ is aryl, which may be optionally substituted with one or more substituents selected from the group consisting of hydrogen, lower alkyl, and halo

In yet further embodiments provided herein, X is O.

In other embodiments provided herein,

X is CR₈R₉; and

R₈ and R₉ are each independently hydrogen.

In further embodiments provided herein, m is 0.

In further embodiments provided herein,

Q₁ is N; and

R₂, R₄, R₅, R₆, and R₇ are each independently hydrogen.

In further embodiments provided herein,

X is CR₈R₉; and

R₈ and R₉ are hydrogen.

In yet further embodiments provided herein, R₃ is selected from the group consisting of aryl, cycloalkyl, and arylalkyl, any of which may be optionally substituted.

In certain embodiments, the compounds have structural Formula IV:

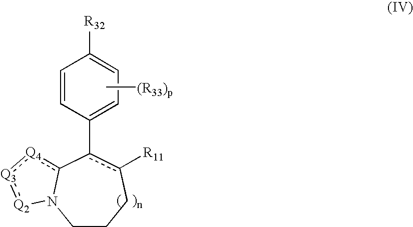

Or a salt, ester, or prodrug thereof, wherein:

Q₂ is selected from the group consisting of N, NR₂₂, CR₂₃, and CR₂₃R₂₄;

Q₃ is selected from the group consisting of N, NR₂₅, CR₂₆, CR₂₆R₂₇, S, and O;

Q₄ is selected from the group consisting of N, NR₂₈, CR₂₉, and CR₂₉R₃₀;

n is an integer from 0 to 2;

p is an integer from 0 to 4;

R₁₁ is selected from the group consisting of —C(O)NR₁₅R₁₆, —NR₁₇C(O)NR₁₈R₁₉, and, —NR₂₀C(O)OR₂₁;

R₁₅, R₁₇, R₁₉, R₂₀, R₂₂, R₂₅, and R₂₈ are each independently selected from the group consisting of hydrogen, null, and lower alkyl; and

R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and

each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

In further embodiments provided herein,

Q₂ is selected from the group consisting of N, and CR₂₃;

Q₃ is selected from the group consisting of N and CR₂₆;

Q₄ is selected from the group consisting of N and CR₂₉;

the optional double bonds between Q₂ and Q₃, and between Q₄ and the adjacent carbon, are each present;

the optional double bond between Q₃ and Q₄ is absent; and

R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

In further embodiments provided herein,

n is an integer from 0 to 1; and

p is 0.

In further embodiments provided herein,

R₁₅, R₁₇, and R₁₉, and R₂₀ are each independently hydrogen; and

R₁₆, R₁₈, R₂₀, and R₂₁ are each independently selected from the group consisting of aryl and arylalkyl, any of which may be optionally substituted with a substituent selected from the group consisting of hydrogen, alkoxy, lower alkyl, halo, perhaloalkoxy, and perhaloalkyl.

In further embodiments provided herein,

Q₂ is CR₂₃;

Q₃ is CR₂₆;

Q₄ is CR₂₉;

R₂₃, R₂₆, and R₂₉ are each independently hydrogen; and

R₃₂ is selected from the group consisting of hydrogen, lower alkyl, alkoxy, cyanoalkyl, and haloalkyl.

In further embodiments provided herein,

Q₂ is CR₂₃;

Q₃ is CR₂₆;

Q₄is N;

R₂₃ and R₂₆ are each independently hydrogen; and

R₃₂ is selected from the group consisting of hydrogen, lower alkyl, alkoxy, cyanoalkyl, and haloalkyl.

In further embodiments provided herein,

Q₂ is CR₂₃;

Q₃ is N;

Q₄ is CR₂₉;

R₂₃ and R₂₉ are each independently hydrogen; and

R₃₂ is selected from the group consisting of hydrogen, lower alkyl, alkoxy, cyanoalkyl, and haloalkyl.

In further embodiments provided herein,

Q₂ is N;

Q₃ is CR₂₃;

Q₄ is CR₂₄;

R₂₃ and R₂₄ are each independently hydrogen; and

R₂₅ is selected from the group consisting of hydrogen, lower alkyl, alkoxy, cyanoalkyl, and haloalkyl.

In certain embodiments, the compounds have structural Formula V:

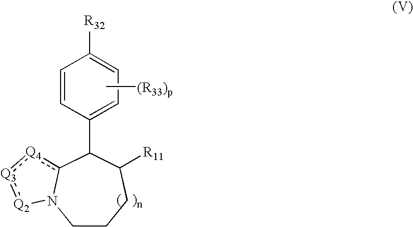

Or a salt, ester, or prodrug thereof, wherein:

Q₂ is selected from the group consisting of N, NR₂₂, CR₂₃, and CR₂₃R₂₄;

Q₃ is selected from the group consisting of N, NR₂₅, CR₂₆, CR₂₆R₂₇, S, and O;

Q₄ is selected from the group consisting of N, NR₂₈, CR₂₉, and CR₂₉R₃₀;

n is an integer from 0 to 2;

p is an integer from 0 to 4;

R₁₁ is selected from the group consisting of —C(O)NR₁₅R₁₆, —NR₁₇C(O)NR₁₈R₁₉, and, —NR₂₀C(O)OR₂₁;

R₁₅, R₁₇, R₁₉, R₂₀, R₂₂, R₂₅, and R₂₈ are each independently selected from the group consisting of hydrogen, null, and lower alkyl; and

R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and

each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

In further embodiments provided herein,

Q₂ is selected from the group consisting of N, and CR₂₃;

Q₃ is selected from the group consisting of N and CR₂₆;

Q₄ is selected from the group consisting of N and CR₂₉;

the optional double bonds between Q₂ and Q₃ and Q₄ and the adjacent carbon are each present;

the optional double bond between Q₃ and Q₄ is absent; and

R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

In further embodiments provided herein,

n is an integer from 0 to 1 ; and

p is 0.

In further embodiments provided herein,

R₁₅, R₁₇, and R₁₉ are each independently hydrogen; and

R₁₆, R₁₈, R₂₀, and R₂₁ are each independently selected from the group consisting of aryl and arylalkyl, any of which may be optionally substituted with a substituent selected from the group consisting of hydrogen, alkoxy, lower alkyl, halo, perhaloalkoxy, and perhaloalkyl.

In further embodiments provided herein,

Q₂ is CR₂₃;

Q₃ is CR₂₆;

Q₄ is CR₂₉;

R₂₃, R₂₆, and R₂₉ are each independently hydrogen; and

R₃₂ is selected from the group consisting of hydrogen, lower alkyl, alkoxy, cyanoalkyl, and haloalkyl.

In further embodiments provided herein,

Q₂ is CR₂₃;

Q₃ is CR₂₆;

Q₄ is N;

R₂₃ and R₂₆ are each independently hydrogen; and

R₃₂ is selected from the group consisting of hydrogen, lower alkyl, alkoxy, cyanoalkyl, and haloalkyl.

In further embodiments provided herein,

Q₂ is N;

Q₃ is CR₂₃;

Q₄ is CR₂₄;

R₂₃ and R₂₄ are each independently hydrogen; and

R₂₅ is selected from the group consisting of hydrogen, lower alkyl, alkoxy, cyanoalkyl, and haloalkyl.

In yet further embodiments provided herein,

Y is NR₁₀;

R₁₀ is selected from the group consisting of aryl, arylalkyl, heteroaryl, cycloalkyl, heterocycloalkyl, —C(O)R₁₄, —C(O)NR₁₅R₁₆, any of which may be optionally substituted.

In other embodiments provided herein,

R₁₀ is selected from the group consisting of aryl, —C(O)R₁₄, —C(O)NR₁₅R₁₆, any of which may be optionally substituted.

In certain embodiments, the compounds have structural Formula VI

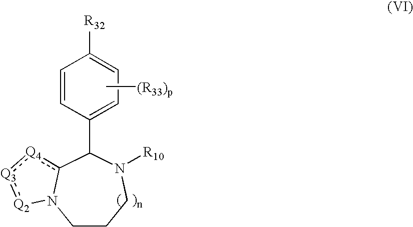

Or a salt, ester, or prodrug thereof, wherein:

Q₂ is selected from the group consisting of N, NR₂₂, CR₂₃, and CR₂₃R₂₄;

Q₃ is selected from the group consisting of N, NR₂₅, CR₂₆, CR₂₆R₂₇, S, and O;

Q₄ is selected from the group consisting of N, NR₂₈, CR₂₉, and CR₂₉R₃₀;

n is an integer from 0 to 2;

p is an integer from 0 to 4;

R₁₀ is selected from the group consisting of —C(O)R₁₄, —C(O)NR₁₅R₁₆, and aryl, which may be optionally substituted;

R₁₅, R₁₇, R₁₉, R₂₀, R₂₂, R₂₅, and R₂₈ are each independently selected from the group consisting of hydrogen, null, and lower alkyl;

R₁₄, R₁₆, R₁₈, R₂₁, R₂₃, R₂₄, R₂₆, R₂₇, R₂₉, and R₃₀ are each independent selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;

R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and

each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

In further embodiments provided herein,

Q₂ is selected from the group consisting of N and CR₂₃;

Q₃ is selected from the group consisting of N and CR₂₆;

Q₄ is selected from the group consisting of N and CR₂₉;

the optional double bonds between Q₂ and Q₃ and Q₄ and the adjacent carbon are each present;

the optional double bond between Q₃ and Q₄ is absent; and

R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

In further embodiments provided herein,

n is an integer from 0 to 1;

p is 0;

R₁₄ and R₁₆ are each independently selected from the group consisting of aryl, arylalkyl, heteroaryl, any of which may be optionally substituted; and

R₁₅ is hydrogen;

R₃₂ is independently selected from the group consisting of hydrogen, lower alkyl, alkoxy,cyanoalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and

each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

In yet further embodiments provided herein, n is 1.

In further embodiments provided herein, R₁₀ is —C(O)R₁₄.

In other embodiments provided herein,

Q₂ is CR₂₃;

Q₃ is CR₂₆;

Q₄ is CR₂₉;

R₂₃, R₂₆, and R₂₉ are each independently hydrogen; and

R₃₂ is selected from the group consisting of hydrogen, lower alkyl, alkoxy, cyanoalkyl, and haloalkyl.

In certain embodiments provided herein,

Q₂ is CR₂₃;

Q₃ is CR₂₆;

Q₄ is N;

R₂₃ and R₂₆ are each independently hydrogen; and

R₃₂ is selected from the group consisting of hydrogen, lower alkyl, alkoxy, cyanoalkyl, and haloalkyl.

In further embodiments provided herein,

Q₂ is CR₂₃;

Q₃ is N;

Q₄ is CR₂₉;

R₂₃ and R₂₉ are each independently hydrogen; and

R₃₂ is selected from the group consisting of hydrogen, lower alkyl, alkoxy, cyanoalkyl, and haloalkyl.

In yet further embodiments provided herein,

Q₂ is N;

Q₃ is CR₂₃;

Q₄ is CR₂₄;

R₂₃ and R₂₄ are each independently hydrogen; and

R₂₅ is selected from the group consisting of hydrogen, lower alkyl, alkoxy, cyanoalkyl, and haloalkyl.

In other embodiments provided herein, R₂, R₄, R₅, R₆, and R₇ are hydrogen.

In certain embodiments provided herein,

X is CR₈R₉; and

R₈ and R₉ are each independently hydrogen.

In certain embodiments provided herein,

Y is NR₁₀; and

R₃ is selected from the group consisting of aryl and arylalkyl, any of which may be optionally substituted.

In certain embodiments provided herein, Q₁ is N.

In other embodiments provided herein, R₃ is aryl, which may be optionally substituted in the para-position with a substituent selected from the group consisting of hydrogen, lower alkyl, alkoxy, cyanoalkyl, and haloalkyl.

In further embodiments provided herein,

R₁₀ is selected from the group consisting of —C(O)R₁₄ and —C(O)NR₁₅R₁₆;

R₁₄ and R₁₆ are each independently selected from the group consisting of lower alkyl, aryl, and arylalkyl, any of which may be optionally substituted; and

R₁₅ is hydrogen.

In further embodiments provided herein,

n is an integer from 0 to 1;

m is 0; and

the optional double bonds between Q₁ and Q₂, Q₂ and Q₃, and Q₃ and Q₄ are each absent.

In yet further embodiments provided herein,

Q₂ is CR₂₃R₂₄;

Q₃ is selected from the group consisting of NR₂₂, CR₂₆R₂₇, S, and O;

Q₄ is CR₂₉R₃₀;

R₂₂ is selected from the group consisting of hydrogen and lower alkyl; and

R₂₃, R₂₄, R₂₆, R₂₇, R₂₉, and R₃₀ are each independently selected from the group consisting of hydrogen, lower alkyl, alkenyl, and alkynyl, any of which may be optionally substituted.

In further embodiments provided herein,

Q₃ is NR₂₂; and

NR₂₂ is selected from the group consisting of hydrogen and lower alkyl.

In other embodiments provided herein, n is 0.

In other embodiments provided herein, n is 1.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms,as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“CB2 modulator” is used herein to refer to a compound that exhibits an EC₅₀ with respect to CB2 activity of no more than about 100 μM and more typically not more than about 50 μM, as measured in the CB2 radioligand binding assay and CB2 GTPγ[³⁵S] functional assay_described generally hereinbelow. “EC₅₀” is that concentration of modulator which activates an enzyme (e.g., CB2) to half-maximal level. Certain compounds disclosed herein have been discovered to exhibit modulatory activity against CB2. In certain embodiments, compounds will exhibit an EC₅₀ with respect to CB2 of no more than about 10 μM; in further embodiments, compounds will exhibit an EC₅₀ with respect to CB2 of no more than about 5 μM; in yet further embodiments, compounds will exhibit an EC₅₀ with respect to CB2 of not more than about 1 μM; in yet further embodiments, compounds will exhibit an EC₅₀ with respect to CB2 of not more than about 200 nM, as measured in the CB2 assay described herein. In certain embodiments, said modulators are agonists. The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate(isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6)alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds disclosed herein with: a) corticosteroids including betamethasone dipropionate (augmented and nonaugmented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocaine, and EMLA Cream [Eutectic Mixture of Local Anesthetics (lidocaine 2.5% and prilocaine 2.5%; an emulsion in which the oil phase is a eutectic mixture of lidocaine and prilocaine in a ratio of 1:1 by weight. This eutectic mixture has a melting point below room temperature and therefore both local anesthetics exist as a liquid oil rather then as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g, duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) other cannabinoids including selective and non-selective CB1/CB2 ligands; and other agents, such as capsaicin.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating CB2-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of CB2-mediated disorders.

The compounds disclosed herein are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds are also useful in the treatment of pain associated with acute herpes zoster (shingles), postherpetic neuralgia (PHN), and associated pain syndromes such as ocular pain. The compounds are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds are also useful for the treatment of pain-related disorders such as tactile allodynia and hyperalgesia. The pain may be somatogenic (either nociceptive or neuropathic), acute and/or chronic.

Furthermore, the compounds disclosed herein can be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods disclosed herein are useful in the treatment or prevention of drug withdrawal symptoms, for example treatment or prevention of symptoms of withdrawal from opiate, alcohol, or tobacco addiction.

Other disorders or conditions which can be advantageously treated by the compounds disclosed herein include inflammation. The compounds disclosed herein are useful as anti-inflammatory agents with the additional benefit of having significantly less harmful side effects. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as reflux esophagitis, diarrhea, inflammatory bowel disease, Crohn's disease, gastritis, irritable bowel syndrome and ulcerative colitis. The compounds may also be used in the treatment of pulmonary inflammation, such as that associated with viral infections and cystic fibrosis. In addition, compounds disclosed herein are also useful in organ transplant patients either alone or in combination with conventional immunomodulators. Yet further, the compounds disclosed herein are useful in the treatment of pruritis and vitaligo. In addition, the compounds can be used to treat insulin resistance and other metabolic disorders such as atherosclerosis that are typically associated with an exaggerated inflammatory signaling.

The compounds disclosed herein can be used in the treatment ischemia, retinitis of ophthalmic diseases, such as glaucoma, retinal ganglion degeneration, ocular irritation, retinopathies, uveitis, ocular photophobia, and of inflammation and pain associated with acute injury to the eye tissue. Specifically, the compounds can be used to treat glaucomatous retinopathy and/or diabetic retinopathy. The compounds can also be used to treat post-operative inflammation or pain as from ophthalmic surgery such as cataract surgery and refractive surgery.

Still other disorders or conditions advantageously treated by the compounds disclosed herein include the prevention or treatment of hypreproliferative diseases, especially cancers. Hematological and non-hematological malignancies which may be treated or prevented include but are not limited to multiple myeloma, acute and chronic leukemias including Acute Lymphocytic Leukemia (ALL), Chronic Lymphocytic Leukemia (CLL), and Chronic Myelogenous Leukemia (CLL), lymphomas, including Hodgkin's lymphoma and non-Hodgkin's lymphoma (low, intermediate, and high grade), as well as solid tumors and malignancies of the brain, head and neck, breast, lung, reproductive tract, upper digestive tract, pancreas, liver, renal, bladder, prostate and colorectal. The compounds can also be used to treat fibrosis, such as that which occurs with radiation therapy. The present compounds can also be used to treat subjects having adenomatous polyps, including those with familial adenomatous polyposis (FAP). Additionally, the present compounds can be used to prevent polyps from forming in patients at risk of FAP. The compounds may also be used to treat malignancies of the skin including, but not limited to, melanomas.

The compounds disclosed herein may also be used in the treatment of autoimmune diseases including but not limited to multiple sclerosis, rheumatoid arthritis, systemic lupus erythematosus, myasthenia gravis, diabetes mellitus type I, inflammatory bowel disease or irritable bowel syndrome, psoriasis and other immune related disorders including but not limited to tissue rejection in organ transplants, malabsorption syndromes such as celiac disease, pulmonary diseases such as asthma and Sjogren's syndrome.

Besides being useful for human treatment, certain compounds and formulations, disclosed herein may also be useful for veterinary treatment of companion animals, exotic animals and farm animals, including mammals, rodents, rabbits, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Synthetic Methods for Preparing Compounds

The following schemes can be used to practice the present invention.

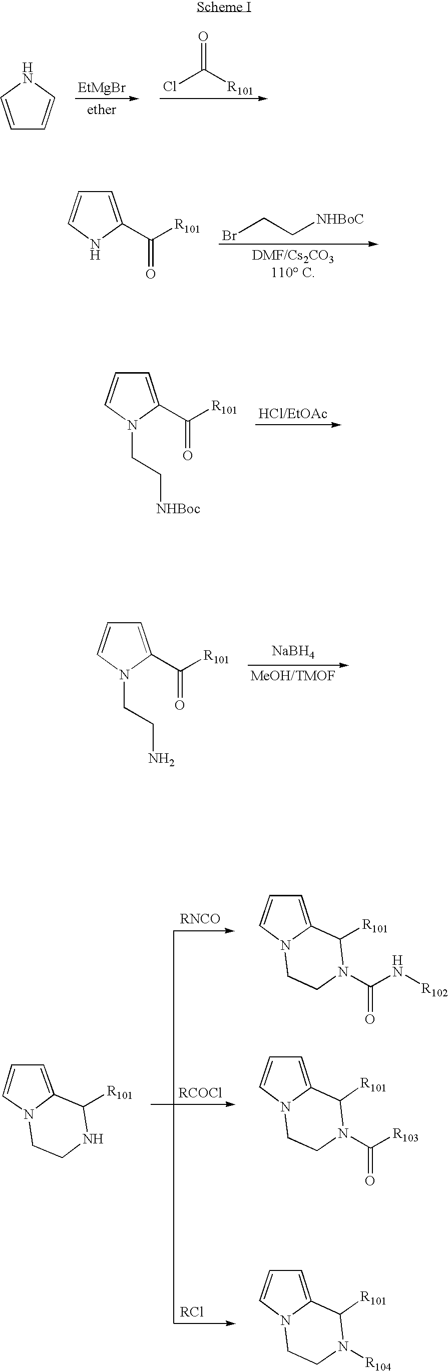

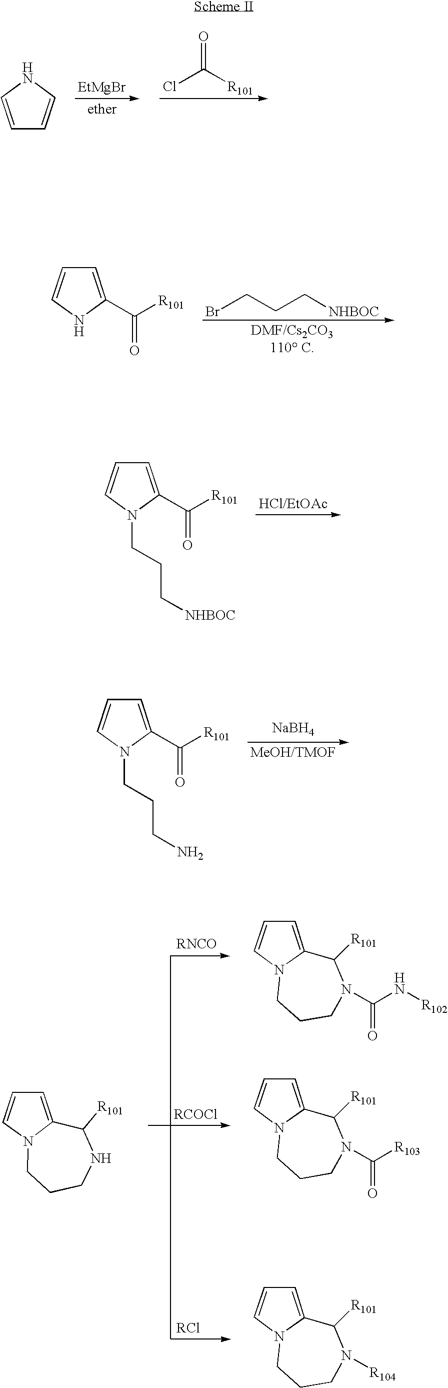

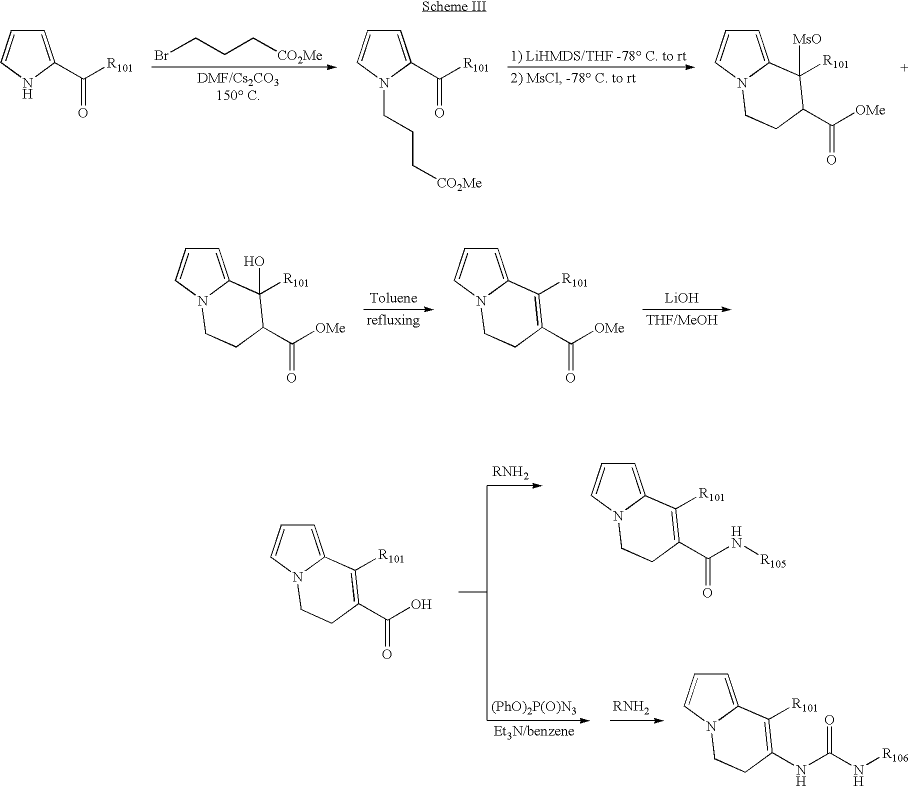

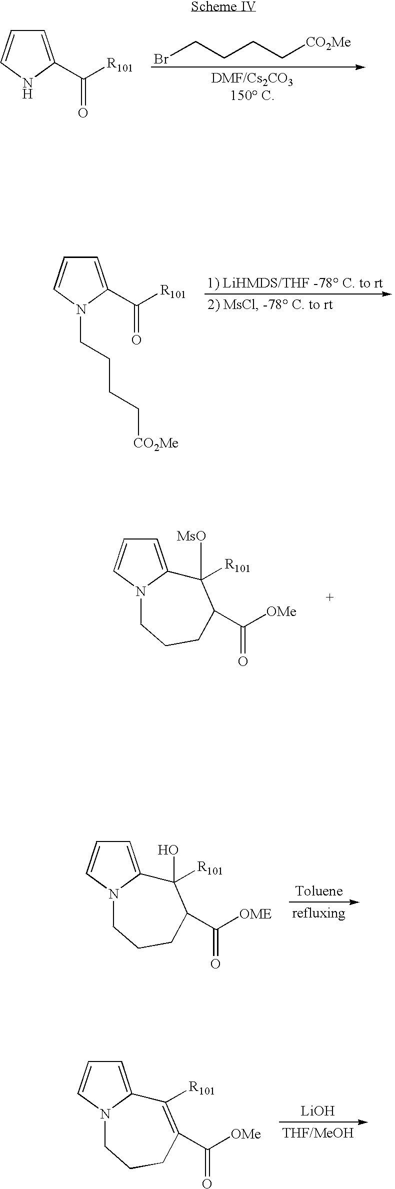

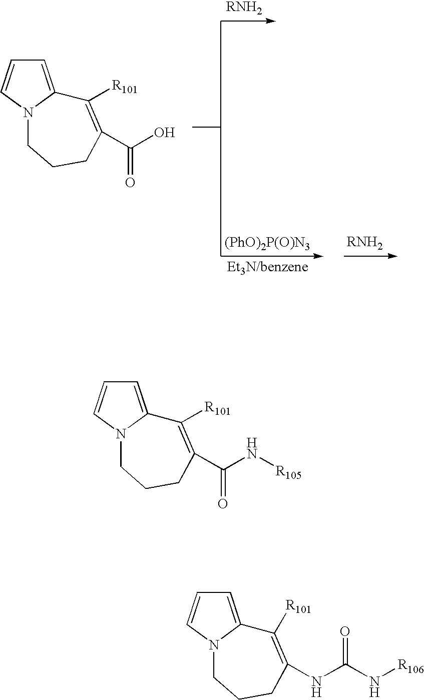

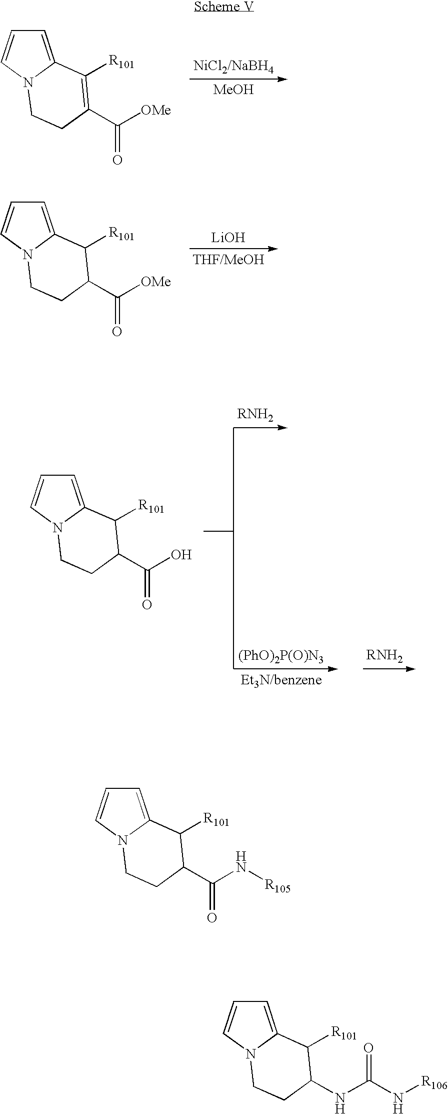

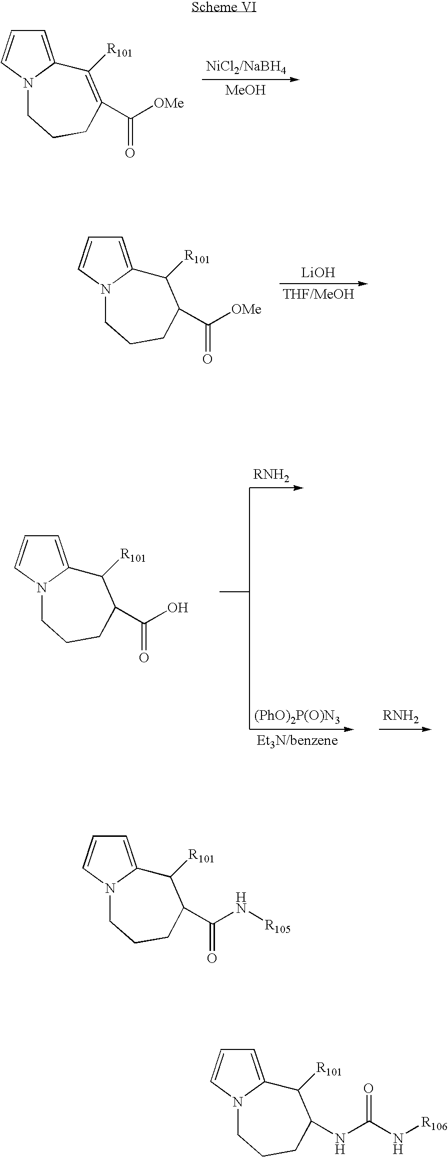

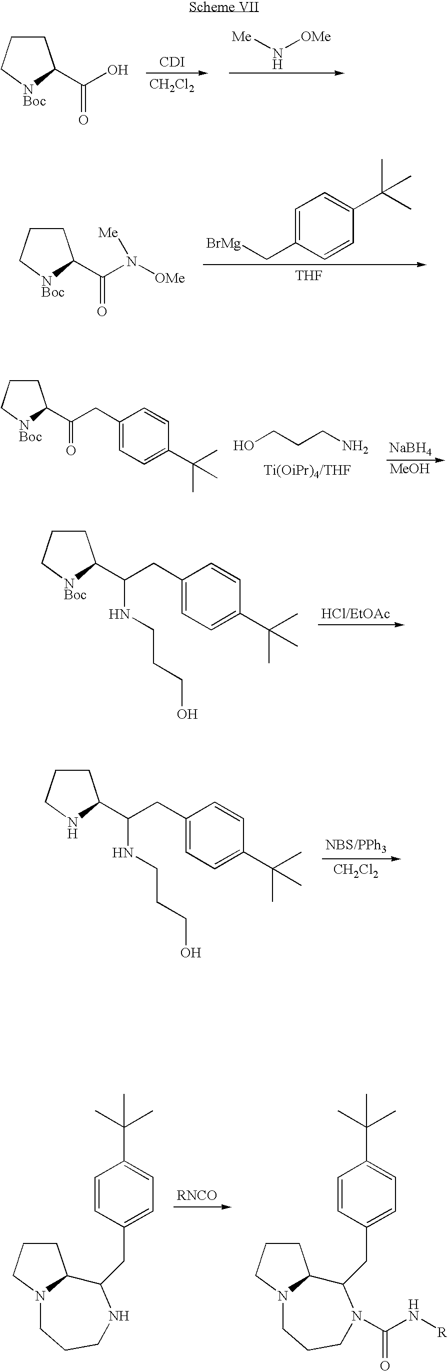

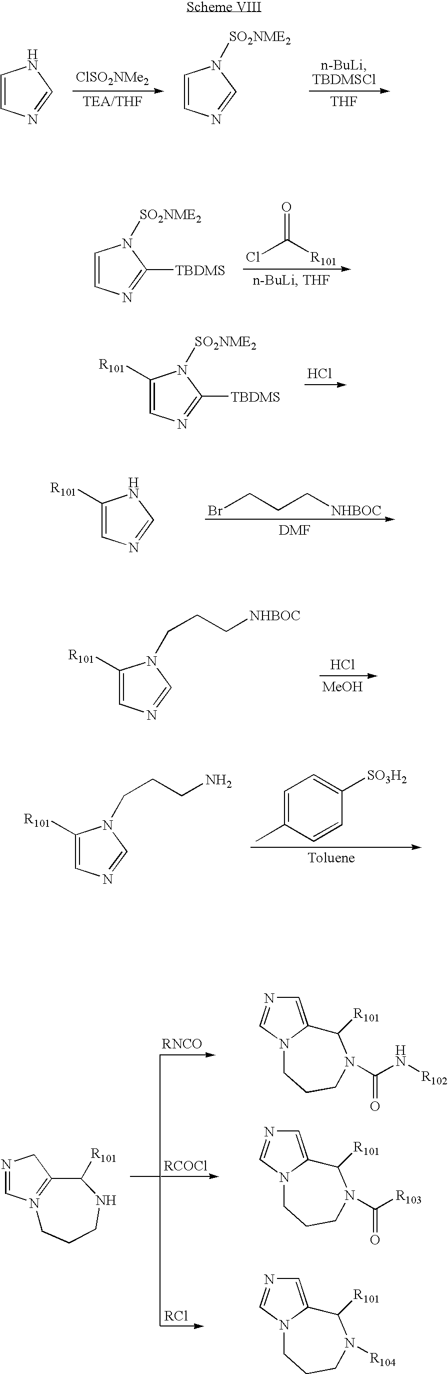

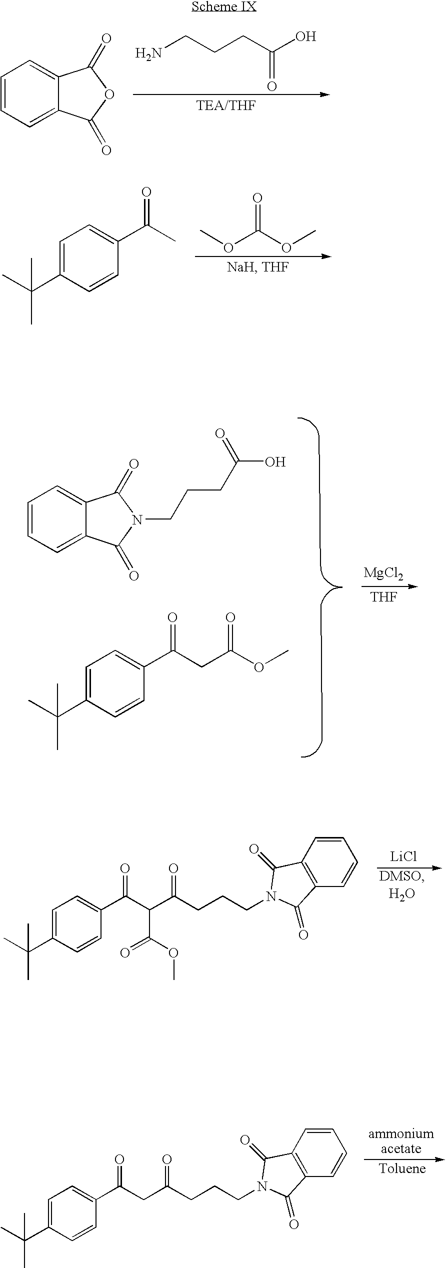

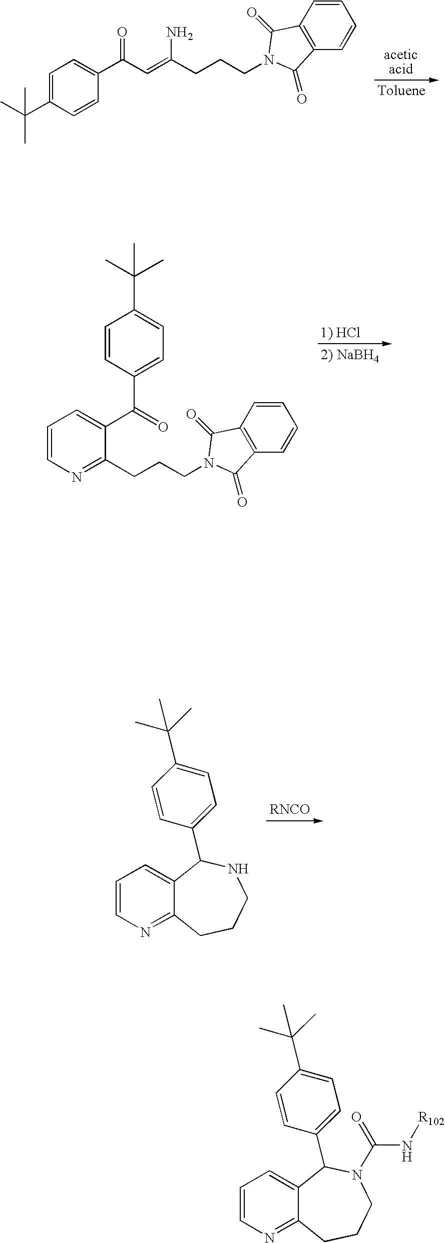

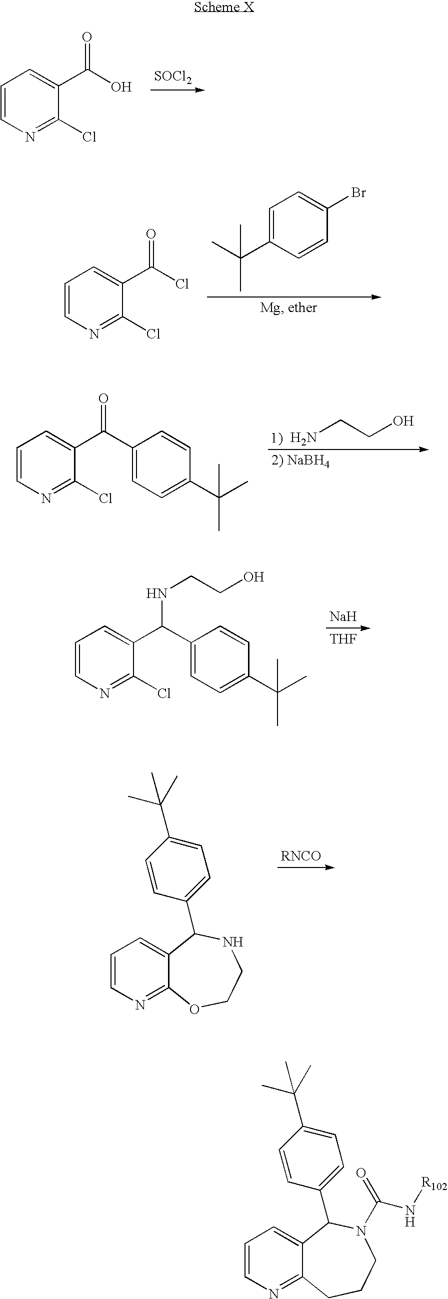

The invention is further illustrated by the following examples. All IUPAC names were generated using CambridgeSoft's ChemDraw 10.0.

### EXAMPLE 1

### 1-(4-tert-Butylphenyl)-1,2,3,4-tetrahydropyrrolo[1,2-a]pyrazine

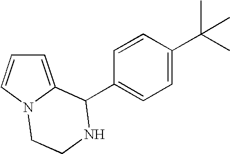

### Step 1

To a solution of ethyl magnesium bromide/THF (1.0 M, 30 mL) was slowly added a solution of pyrrole (2.1 mL, 30.0 mmol) in ether (15 mL). The mixture was stirred at room temperature for 30 minutes then 4-tert-butylbenzoyl chloride (7.04 g, 35.8 mmol) was slowly added to the mixture at room temperature. The mixture was stirred overnight. The reaction mixture was diluted with ethyl acetate, washed with saturated NH₄Cl, water, brine, dried over Na₂SO₄, and concentrated in vacuo. The crude material was purified by silica gel chromatography to give 3.48 g (51% yield) of (4-tert-butylphenyl)(1H-pyrrol-2-yl)methanone. ¹H NMR (400 MHz, CDCl₃) δ 9.82 (b, 1H), 7.87 (d, 2H), 7.50 (d, 2H), 7.15 (m, 1H), 6.93(m, 1H), 6.34 (m, 1H), 1.37 (s, 9H).

### Step 2

A mixture of (4-tert-butylphenyl)(1H-pyrrol-2-yl)methanone (2.84 g, 12.5 mmol), tert-butyl 2-bromoethylcarbamate (2.8 g, 12.5 mmol) and Cs₂CO₃ (6.1 g, 18.8 mmol) in DMF 950 mL) was heated to 120° C. with stirring. The mixture was stirred at 120° C. for 12 hours and then diluted with water. The resulting mixture was extracted with ethyl acetate. The organic layer was washed with brine, dried over Na₂SO₄ and concentrated in vacuo. The crude material was purified by silica gel chromatography to give 3.63 g (78% yield) of tert-butyl 2-(2-(4-tert-butylbenzoyl)-1H-pyrrol-1-yl)ethylcarbamate. ¹H NMR (400 MHz, CDCl₃) δ 7.74 (d, 2H), 7.46 (d, 2H), 6.97 (m, 1H), 6.79 (m, 1H), 6.18 (m, 1H), 4.49 (t, 2H), 3.55 (t, 2H), 1.42 (s, 9H), 1.36 (s, 9H).

### Step 3

A mixture of tert-butyl 2-(2-(4-tert-butylbenzoyl)-1H-pyrrol-1-yl)ethylcarbamate (680 mg, 1.83 mmol) and concentrated HCl (2 ML) in ethyl acetate (20 mL) was stirred at room temperature for 5 hours. The mixture was diluted with saturated NaHCO₃. After separation, the aqueous solution was extracted with ethyl acetate and the combined organic layers were washed with water, brine, dried over Na₂SO₄ and concentrated in vacuo to give 497 mg (99% yield) of (1-(2-aminoethyl)-1H-pyrrol-2-yl)(4-tert-butylphenyl)methanone. ¹H NMR (400 MHz, CDCl₃) δ 7.78 (d, 2H), 7.49 (d, 2H), 7.02 (m, 1H), 6.67 (m, 1H), 6.33 (m, 1H), 4.17 (t, 2H), 4.08 (t, 2H), 1.33 (s, 9H).

### Step 4

A solution of (1-(2-aminoethyl)-1H-pyrrol-2-yl)(4-tert-butylphenyl)methanone (1.34 g, 4.97 mmol) in MeOH/TMOF (3:1, 80 mL) was stirred at room temperature for 30 minutes. NaBH₄ (376 mg, 10.0 mmol) was added to the solution in portions and stirred 3.5 hours. The reaction mixture was concentrated in vacuo and the residue taken up in 3 N NaOH (50 mL). The aqueous mixture was extracted with ethyl acetate. The organic layer was washed with water, brine, dried over Na₂SO₄, and concentrated in vacuo to give 1.08 g (86% yield) of the title compound as a white solid. ¹H NMR (400 MHz, CDCl₃) δ 7.34 (m, 4H), 6.60 (m, 1H), 6.12 (m, 1H), 5.60 (m, 1H), 5.10 (s, 1H), 4.10 (m, 1H), 4.01 (m, 1H), 3.35 (m, 1H), 3.25 (m, 1H), 1.31 (s, 9H).

### EXAMPLE 2

### 1-(4-tert-Butylphenyl)-N-(2,4-difluorophenyl)-3,4-dihydropyrrolo[1,2-a]pyrazine-2(1H)-carboxamide

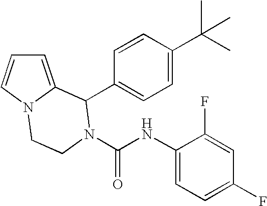

A solution of 1-(4-tert-butylphenyl)-1,2,3,4-tetrahydropyrrolo[1,2-a]pyrazine (123 mg, 0.48 mmol) and 2,4-difluorophenylisocyanate (57.3 μL, 0.48 mmol) in THF (5 mL) was stirred for 2 hours. The mixture was concentrated in vacuo to give the title compound. ¹H NMR (400 MHz, CDCl₃) δ 7.96 (m, 1H), 7.39 (d, 2H), 7.30 (d, 2H), 6.80 (m, 2H), 6.65 (m, 1H), 6.55 (d, 1H), 6.15 (m, 2H), 5.90 (m, 1H), 4.43 (m, 1H), 4.10 (m, 2H), 3.68 (m, 1H), 1.31 (s, 9H).

### EXAMPLE 3

### 1-(4-tert-Butylphenyl)-N-(2,4-dichlorophenyl)-3,4-dihydropyrrolo[1,2-a]pyrazine-2(1H)-carboxamide

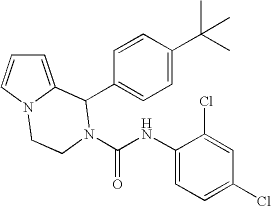

The compound 1-(4-tert-butylphenyl)-N-(2,4-dichlorophenyl)-3,4-dihydropyrrolo[1,2-a]pyrazine-2(1H)-carboxamide was prepared following the procedures described for Example 2 using 2,4-dichlorophenylisocyanate. ¹H NMR (400 MHz, DMSO) δ 8.45 (s, 1H), 7.61 (d, 1H), 7.54 (d, 1H), 7.38 (dd, 1H), 7.35 (d, 2H), 7.21 (d, 2H), 6.76 (m, 1H), 6.52 (s, 1H), 6.07 (t, 1H), 6.15 (m, 2H), 5.92 (m, 1H), 4.16 (m, 1H), 4.03 (m, 2H), 3.46 (m, 1H), 1.26 (s, 9H).

### EXAMPLE 4

### 1-Phenyl-1,2,3,4-tetrahydropyrrolo[1,2-a]pyrazine

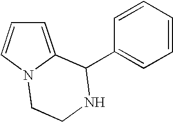

The compound 1-phenyl-1,2,3,4-tetrahydropyrrolo[1,2-a]pyrazine was prepared following the procedures described for Example 2 using benzoyl chloride. ¹H NMR (400 MHz, CDCl₃) δ 7.33 (m, 5H), 6.54 (m, 1H), 6.05 (m, 1H), 5.51 (m, 1H), 5.07 (s, 1H), 4.08 (m, 1H), 3.28 (m, 1H), 3.18 (m, 1H), 1.77 (m, 1H), 1.59 (m, 1H), 1.27 (s, 9H).

### EXAMPLE 5

### N-(2,4-Difluorophenyl)-1-phenyl-3,4-dihydropyrrolo[1,2-a]pyrazine-2(1H)-carboxamide

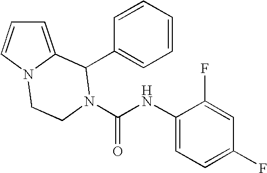

The compound N-(2,4-difluorophenyl)-1-phenyl-3,4-dihydropyrrolo[1,2-a]pyrazine-2(1H)-carboxamide was prepared following the procedures described for Example 2 using 2,4-difluorophenylisocyante. ¹H NMR (400 MHz, DMSO) δ 8.58 (s, 1H), 7.41 (dt, 1H), 7.38 (m, 2H), 7.27 (m, 4H), 7.03 (m, 1H), 6.77 (dd, 1H), 6.56 (s, 1H), 6.09 (t, 1H), 5.94 (dd, 1H), 4.16 (td, 1H), 4.02 (m, 2H), 3.40 (m, 1H)

### EXAMPLE 6

### 1-(4-tert-Butylphenyl)-2,3,4,5-tetrahydro-1H-pyrrolo[1,2-a][1,4]diazepine

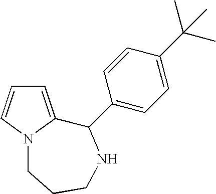

The compound 1-(4-tert-butylphenyl)-2,3,4,5-tetrahydro-1H-pyrrolo[1,2-a][1,4]diazepine was prepared following the procedures described for Example 1 using tert-butyl 2-bromopropylcarbamate. ¹H NMR (400 MHz, DMSO) δ 7.33 (d, 2H), 7.22 (d, 2H), 6.61 (s, 1H), 5.66 (m, 1H), 5.03 (s, 1H), 4.78 (s, 1H), 4.04 (m, 2H), 3.15 (m, 1H), 2.87 (t, 1H), 1.77 (m, 1H), 1.59 (m, 1H), 1.27 (s, 9H).

### EXAMPLE 7

### 1-(4-tert-Butylphenyl)-N-(2,4-difluorophenyl)-4,5-dihydro-1H-pyrrolo[1,2-a][1,4]diazepine-2(3H)-carboxamide

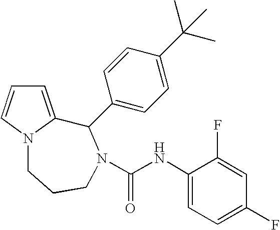

The compound 1-(4-tert-butylphenyl)-N-(2,4-difluorophenyl)-4,5-dihydro-1H-pyrrolo[1,2-a][1,4]diazepine-2(3H)-carboxamide was prepared following the procedures described for Example 2 using 1-(4-tert-butylphenyl)-2,3,4,5-tetrahydro-1H-pyrrolo[1,2-a][1,4]diazepine and 2,4-difluorophenylisocyanate. ¹H NMR (400 MHz, CDCl₃) δ 8.03 (dt, 1H), 7.42 (d, 2H), 7.22 (d, 2H), 6.80 (m, 1H), 6.75 (m, 1H), 6.66(t, 1H), 6.63 (d, 1H), 6.12 (m, 1H), 6.02 (t, 1H), 5.65 (m, 1H), 4.25 (m, 1H), 4.05 (m, 2H), 3.06 (m, 1H), 2.13 (m, 1H), 1.80 (m, 1H), 1.34 (s, 9H).

### EXAMPLE 8

### N-(2,4-Difluorophenyl)-1-p-tolyl-4,5-dihydro-1H-pyrrolo[1,2-a][1,4]diazepine-2(3H)-carboxamide

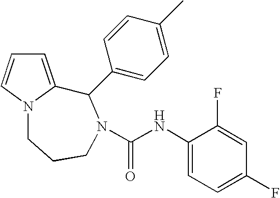

The compound N-(2,4-difluorophenyl)-1-p-tolyl-4,5-dihydro-1H-pyrrolo[1,2-a][1,4]diazepine-2(3H)-carboxamide was prepared following the procedures described for Example 2 using 2,4-difluorophenylisocyanate. ¹H NMR (400 MHz, CDCl₃) δ 8.28 (s, 1H), 7.42 (dt, 1H), 7.23 (m, 1H), 7.17 (d, 2H), 6.99 (m, 1H), 6.90 (d, 2H), 6.75 (t, 1H), 6.61 (s, 1H), 5.88 (t, 1H), 5.77 (m, 1H), 4.07 (m, 2H), 3.77 (m, 1H), 2.93 (m, 1H), 2.28 (s, 3H), 1.76 (m, 2H).

### EXAMPLE 9

### N-(2,4-Dichlorophenyl)-1-p-tolyl-4,5-dihydro-1H-pyrrolo[1,2-a][1,4]diazepine-2(3H)-carboxamide

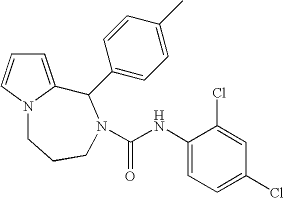

The compound N-(2,4-dichlorophenyl)-1-p-tolyl-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.09 (b, 1H), 7.30 (m, 1H), 7.57 (d, 1H), 7.35 (dd, 1H), 7.19 (d, 2H), 6.98 (d, 2H), 6.76 (t, 1H), 6.55 (s, 1H), 5.87 (t, 1H), 5.67 (s, 1H), 4.10 (m, 2H), 3.84 (m, 1H), 2.92 (m, 1H), 2.29 (s, 3H), 1.82 (m, 1H), 1.75 (m, 1H).

### EXAMPLE 10

### N-(2,4-Difluorophenyl)-1-m-tolyl-4,5-dihydro-1H-pyrrolo[1,2-a][1,4]diazepine-2(3H)-carboxamide

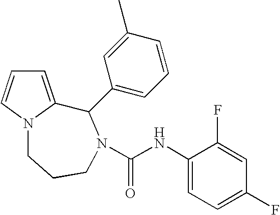

The compound N-(2,4-difluorophenyl)-1-m-tolyl-4,5-dihydro-1H-pyrrolo[1,2-a][1,4]diazepine-2(3H)-carboxamide was prepared following the procedures described for Example 2 using 2,4-difluorophenylisocyanate. ¹H NMR (400 MHz, CDCl₃) δ 8.09 (dt, 1H), 7.31 (t, 1H), 7.19 (d, 1H), 7.14 (s, 1H), 7.10 (d, 1H), 6.75 (m, 2H), 6.67 (t, 1H), 6.63 (d, 1H), 6.09 (b, 1H), 6.02 (dd, 1H), 5.60 (s, 1H), 4.25 (m, 1H), 4.05 (m, 2H), 3.03 (m, 1H), 2.37 (s, 3H), 2.15 (m, 1H), 1.80 (m, 1H).

### EXAMPLE 11

### N-(2,4-Dichlorophenyl)-1-m-tolyl-4,5-dihydro-1H-pyrrolo[1,2-a][1,4]diazepine-2(3H)-carboxamide

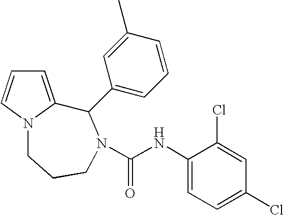

The compound N-(2,4-dichlorophenyl)-1-m-tolyl-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.14 (d, 1H), 7.30 (t, 1H), 7.24 (d, 1H), 7.18 (d, 1H), 7.17 (dd, 1H), 7.14 (s, 1H), 7.11 (d, 1H), 6.98 (s, 1H), 6.66 (t, 1H), 6.11 (b, 1H), 6.09 (dd, 1H). 5.55 (s, 1H), 4.25 (m, 1H), 4.05 (m, 2H), 3.05 (m, 1H), 2.37 (s, 3H), 2.20 (m, 1H), 1.81 (m, 1H).

### EXAMPLE 12

### N-(2,4-Difluorophenyl)-1-phenyl-4,5-dihydro-1H-pyrrolo[1,2-a][1,4]diazepine-2(3H)-carboxamide

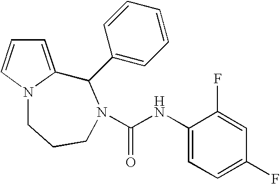

The compound N-(2,4-difluorophenyl)-1-phenyl-4,5-dihydro-1H-pyrrolo[1,2-a][1,4]diazepine-2(3H)-carboxamide was prepared following the procedures described for Example 2 using 2,4-difluorophenylisocyanate. ¹H NMR (400 MHz, DMSO) δ 8.32 (s, 1H), 7.42 (dt, 1H), 7.36 (m, 2H), 7.25 (m, 2H), 7.01 (m, 3H), 6.76 (t, 1H), 6.66 (s, 1H), 5.88 (t, 1H), 5.75 (s, 1H), 4.07 (m, 2H), 3.78 (m, 1H), 2.93 (t, 1H), 1.80 (m, 2H), 1.59 (m, 1H).

### EXAMPLE 13

### N-(2,4-Dichlorophenyl)-1-phenyl-4,5-dihydro-1H-pyrrolo[1,2-a][1,4]diazepine-2(3H)-carboxamide

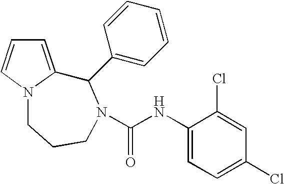

The compound N-(2,4-dichlorophenyl)-1-phenyl-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, DMSO) δ 8.44 (s, 1H), 7.62 (s, 1H), 7.39 (m, 2H), 7.25 (m, 2H), 7.01 (m, 3H), 6.76 (t, 1H), 6.66 (s, 1H), 5.88 (t, 1H), 5.75 (s, 1H), 4.07 (m, 2H), 3.78 (m, 1H), 2.93 (t, 1H), 1.80 (m, 2H), 1.59 (m, 1H).

### EXAMPLE 14

### 1-(4-(Trifluoromethoxy)phenyl)-2,3,4,5-tetrahydro-1H-pyrrolo[1,2-a][1,4]diazepine

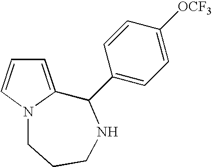

The compound 1-(4-(trifluoromethoxy)phenyl)-2,3,4,5-tetrahydro-1H-pyrrolo[1,2-a][1,4]diazepine was prepared following the procedures described for Example 1 using 4-trifluoromethoxybenzoyl chloride. ¹H NMR (300 MHz, DMSO) δ 7.44 (d, 2H), 7.31 (d, 2H), 6.65 (t, 1H), 5.69 (t, 1H), 5.00 (t, 1H), 4.91 (s, 1H), 4.09 (m, 2H), 3.17 (m, 1H), 2.90 (m, 1H), 2.56 (b, 1H), 1.80 (m, 1H), 1.59 (m, 1H).

### EXAMPLE 15

### N-(2,4-Difluorophenyl)-1-(4-(trifluoromethoxy)phenyl)-4,5-dihydro-1H-pyrrolo[1,2-a][1,4]diazepine-2(3H)-carboxamide

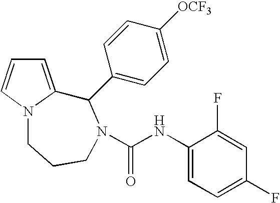

The compound N-(2,4-difluorophenyl)-1-(4-(trifluoromethoxy)phenyl)-4,5-dihydro-1H-pyrrolo[1,2-a][1,4]diazepine-2(3H)-carboxamide was prepared following the procedures described for Example 2 using 1-(4-(trifluoromethoxy)phenyl)-2,3,4,5-tetrahydro-1H-pyrrolo[1,2-a][1,4]diazepine and 2,4-difluorophenylisocyanate. ¹H NMR (400 MHz, CDCl₃) δ 8.01 (dt, 1H), 7.33 (d, 2H), 7.27 (d, 2H), 6.83 (m, 2H), 6.67 (dd, 1H), 6.56 (d, 1H), 6.26 (b, 1H), 6.04 (dd, 1H), 5.67 (b, 1H), 4.19 (m, 1H), 4.10 (m, 1H), 4.00 (m, 1H), 3.08 (t, 1H), 2.14 (m, 1H), 1.84 (m, 1H).

### EXAMPLE 16

### 1-(4-(Trichloromethoxy)phenyl)-2,3,4,5-tetrahydro-1H-pyrrolo[1,2-a][1,4]diazepine

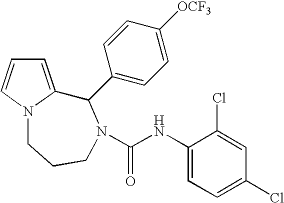

The compound N-(2,4-dichlorophenyl)-1-(4-(trifluoromethoxy)phenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.13 (d, 1H), 7.36 (d, 2H), 7.27 (m, 3H), 7.18 (dd, 1H), 6.94 (s, 1H), 6.68 (dd, 1H), 6.27 (b, 1H), 6.03 (dd, 1H), 5.61 (b, 1H), 4.20 (m, 1H), 4.11 (m, 1H), 4.01 (m, 1H), 3.06 (t, 1H), 2.20 (m, 1H), 1.85 (m, 1H).

### EXAMPLE 17

### 1-(3-tert-Butylphenyl)-2,3,4,5-tetrahydro-1H-pyrrolo[1,2-a][1,4]diazepine

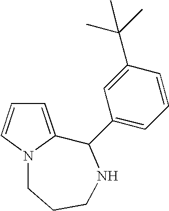

The compound 1-(3-tert-butylphenyl)-2,3,4,5-tetrahydro-1H-pyrrolo[1,2-a][1,4]diazepine was prepared following the procedures described for Example 1 using 3-tert-butylbenzoyl chloride. ¹H NMR (300 MHz, CDCl₃) δ 7.41 (s, 1H), 7.33 (m, 2H), 7.17 (d, 1H), 6.58 (t, 1H), 5.89 (t, 1H), 5.34 (b, 1H), 4.86 (s, 1H), 4.12 (m, 2H), 3.43 (m, 1H), 3.07 (m, 1H), 1.93 (m, 2H), 1.32 (s, 9H).

### EXAMPLE 18

### 1-(3-tert-Butylphenyl)-N-(2,4-difluorophenyl)-4,5-dihydro-1H-pyrrolo[1,2-a][1,4]diazepine-2(3H)-carboxamide

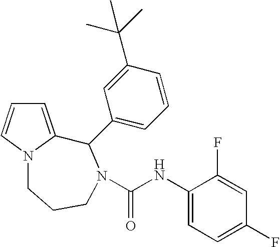

The compound 1-(3-tert-butylphenyl)-N-(2,4-difluorophenyl)-4,5-dihydro-1H-pyrrolo[1,2-a][1,4]diazepine-2(3H)-carboxamide was prepared following the procedures described for Example 2 using 1-(3-tert-butylphenyl)-2,3,4,5-tetrahydro-1H-pyrrolo[1,2-a][1,4]diazepine and 2,4-difluorophenylisocyanate. ¹H NMR (400 MHz, CDCl₃) δ 8.06 (dt, 1H), 7.33(m, 3H), 7.11 (d, 1H), 6.81 (m, 2H), 6.67 (dd, 1H), 6.65 (d, 1H), 6.09 (b, 1H), 6.02 (dd, 1H), 5.55 (b, 1H), 4.28 (m, 1H), 4.09 (m, 2H), 3.03 (m, 1H), 2.17 (m, 1H), 1.83 (m, 1H), 1.32 (s, 9H).

### EXAMPLE 19

### 1-(3-tert-Butylphenyl)-N-(2,4-dichlorophenyl)-4,5-dihydro-1H-pyrrolo[1,2-a][1,4]diazepine-2(3H)-carboxamide

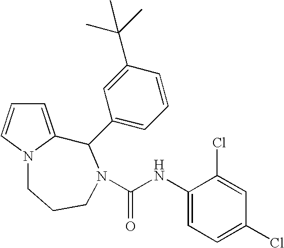

The compound 1-(3-tert-butylphenyl)-N-(2,4-dichlorophenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.14 (d, 1H), 7.36 (m, 3H), 7.22 (d, 1H), 7.17 (dd, 1H), 7.13 (d, 1H), 6.98 (s, 1H), 6.67 (dd, 1H), 6.14 (b, 1H), 6.01 (dd, 1H), 5.49 (b, 1H), 4.27 (m, 1H), 4.10 (m, 2H), 3.00 (t, 1H), 2.20 (m, 1H), 1.81 (m, 1H), 1.32 (s, 9H).

### EXAMPLE 20

### 1-Benzyl-2,3,4,5-tetrahydro-1H-pyrrolo[1,2-a][1,4]diazepine

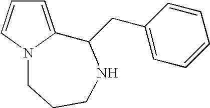

The compound 1-benzyl-2,3,4,5-tetrahydro-1H-pyrrolo[1,2-a][1,4]diazepine was prepared following the procedures described for Example 1 using 2-phenylacetyl chloride. ¹H NMR (300 MHz, DMSO) δ 7.27 (m, 5H), 6.60 (s, 1H), 5.86 (s, 1H), 5.77 (s, 1H), 4.04 (m, 2H), 3.87 (m, 1H), 3.20 (m, 2H), 2.86 (m, 1H), 2.66 (m, 1H), 1.46 (m, 3H).

### EXAMPLE 21

### 1-Benzyl-N-(2,4-dichlorophenyl)-4,5-dihydro-1H-pyrrolo[1,2-a][1,4]diazepine-2(3H)-carboxamide

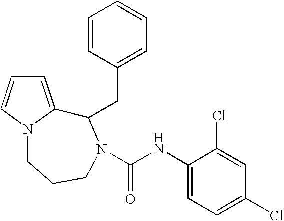

The compound 1-benzyl-N-(2,4-dichlorophenyl)-4,5-dihydro-1H-pyrrolo[1,2-a][1,4]diazepine-2(3H)-carboxamide was prepared following the procedures described for Example 2 using 1-benzyl-2,3,4,5-tetrahydro-1H-pyrrolo[1,2-a][1,4]diazepine and 2,4-dichlorophenylisocyanate. ¹H NMR (400 MHz, CDCl₃) δ 7.97 (d, 1H), 7.27 (m, 6H), 7.16 (dd, 1H), 6.80 (b, 1H), 6.58 (m, 1H), 6.18 (b, 1H), 6.03 (dd, 1H), 4.12 (m, 3H), 3.38 (m, 3H), 2.10 (m, 1H), 1.93 (m, 1H).

### EXAMPLE 22

### 1-(4-tert-Butylbenzyl)-2,3,4,5-tetrahydro-1H-pyrrolo[1,2-a][1,4]diazepine

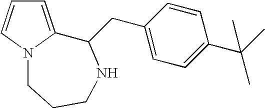

The compound 1-(4-tert-butylbenzyl)-2,3,4,5-tetrahydro-1H-pyrrolo[1,2-a][1,4]diazepine was prepared following the procedures described for Example 1 using 2-(4-tert-butylphenyl)acetyl chloride. ¹H NMR (400 MHz, CDCl₃) δ 7.34 (d, 2H), 7.20 (d, 2H), 6.58 (dd, 1H), 6.13 (m, 1H), 6.01 (dd, 1H), 4.07 (m, 3H), 3.39 (dd, 1H), 3.33 (td, 1H), 3.08 (dd, 1H), 2.89 (dt, 1H), 1.91 (m, 1H), 1.81 (m, 1H) 1.31 (s, 9H).

### EXAMPLE 23

### 1-(4-tert-Butylbenzyl)-N-(2,4-difluorophenyl)-4,5-dihydro-1H-pyrrolo[1,2-a][1,4]diazepine-2(3H)-carboxamide

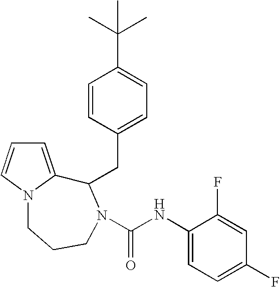

The compound 1-(4-tert-butylbenzyl)-N-(2,4-difluorophenyl)-4,5-dihydro-1H-pyrrolo[1,2-a][1,4]diazepine-2(3H)-carboxamide was prepared following the procedures described for Example 2 using 1-(4-tert-butylbenzyl)-2,3,4,5-tetrahydro-1H-pyrrolo[1,2-a][1,4]diazepine and 2,4-difluorophenylisocyanate. ¹H NMR (400 MHz, CDCl₃) δ 7.64 (b, 1H), 7.33 (d, 2H), 7.19 (d, 2H), 6.75 (m, 2H), 6.59 (m, 1H), 6.20 (b, 1H), 6.07 (dd, 1H), 4.08 (dd, 2H), 3.39 (dd, 2H), 3.16 (m, 2H), 2.16 (m, 1H), 1.87 (m, 2H), (1.25 (s, 9H).

### EXAMPLE 24

### 1-(4-tert-Butylbenzyl)-N-(2,4-dichlorophenyl)-4,5-dihydro-1H-pyrrolo[1,2-a][1,4]diazepine-2(3H)-carboxamide

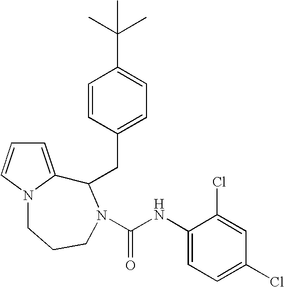

The compound 1-(4-tert-butylbenzyl)-N-(2,4-dichlorophenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 7.92 (d, 1H), 7.29 (d, 2H), 7.28 (d, 1H), 7.18 (d, 2H), 7.14 (dd, 1H), 6.59 (s, 1H), 6.20 (b, 1H), 6.06 (dd, 1H), 4.10 (dd, 2H), 3.38 (dd, 2H), 3.22 (m, 2H), 2.14 (m, 1H), 1.87 (m, 2H), 1.24 (s, 9H).

### EXAMPLE 25

### 4-(4-tert-Butylphenyl)-N-(2,4-dichlorophenyl)-7,8-dihydro-4H-pyrazolo [1,5-a][1,4]diazepine-5(6H)-carboxamide

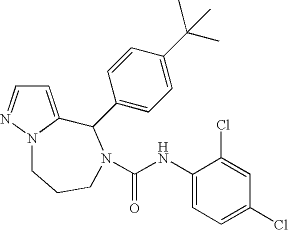

The compound 4-(4-tert-butylphenyl)-N-(2,4-dichlorophenyl)-7,8-dihydro-4H-pyrazolo[1,5-a][1,4]diazepine-5(6H)-carboxamide was prepared following the procedures described for Example 2 using 2,4-dichlorophenylisocyanate. ¹H NMR (400 MHz, CDCl₃) δ 8.14 (d, 1H), 7.43 (d, 2H), 7.39 (d, 1H), 7.28 (d, 1H), 7.21 (dd, 1H), 7.17 (d, 2H), 7.00 (s, 1H), 6.50 (b, 1H), 5.95 (s, 1H), 4.45 (m, 1H), 4.37 (m, 1H), 3.16 (m, 1H), 2.20 (m, 1H), 1.93 (m, 1H), 1.33 (s, 9H).

### EXAMPLE 26

### 9-(4-tert-Butylphenyl)-6,7,8,9-tetrahydro-5H-imidazo[1,2-a][1,4]diazepine

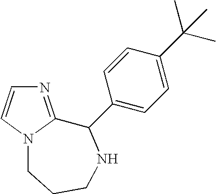

The compound 9-(4-tert-butylphenyl)-6,7,8,9-tetrahydro-5H-imidazo[1,2-a][1,4]diazepine was prepared following the procedures described for Example 1 using 4-tert-butylbenzoyl chloride and imidazole ¹H NMR (300 MHz, CDCl₃) δ 7.39 (d, 2H), 7.21 (d, 2H), 6.94 (s, 1H), 6.88 (s, 1H), 5.30 (s, 1H), 4.08 (m, 2H), 3.30 (m, 1H), 3.15 (m, 1H), 1.89 (m, 2H) 1.33 (s, 9H).

### EXAMPLE 27

### 9-(4-tert-Butylphenyl)-N-(2,4-difluorophenyl)-6,7-dihydro-5H-imidazo [1,2-a][1,4]diazepine-8(9H)-carboxamide

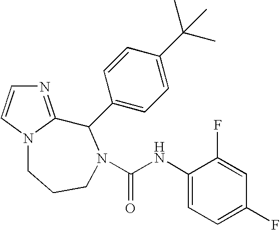

The compound 9-(4-tert-butylphenyl)-N-(2,4-difluorophenyl)-6,7-dihydro-5H-imidazo[1,2-a][1,4]diazepine-8(9H)-carboxamide was prepared following the procedures described for Example 2 using 9-(4-tert-butylphenyl)-6,7,8,9-tetrahydro-5H-imidazo[1,2-a][1,4]diazepine and 2,4-difluorophenylisocyanate. ¹H NMR (400 MHz, CDCl₃) δ 8.03 (dt, 1H), 7.97 (s, 1H), 7.38 (d, 2H), 7.04 (d, 2H), 7.02 (d, 1H), 6.94 (d, 1H), 6.78 (m, 1H), 6.48 (s, 1H), 4.36 (m, 1H), 4.10 (m, 1H), 3.98 (m, 1H), 3.20 (m, 1H), 1.97 (m, 1H), 1.81 (m, 1H), 1.30 (s, 9H).

### EXAMPLE 28

### 9-(4-tert-Butylphenyl)-N-(3,4-difluorophenyl)-6,7-dihydro-5H-imidazo[1,2-a][1,4]diazepine-8(9H)-carboxamide

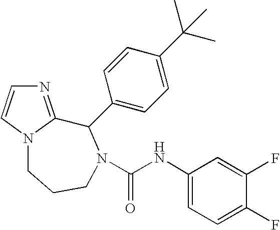

The compound 9-(4-tert-butylphenyl)-N-(3,4-difluorophenyl)-6,7-dihydro-5H-imidazo[1,2-a][1,4]diazepine-8(9H)-carboxamide was prepared following the procedures described for Example 2 using 3,4-difluorophenylisocyanate. ¹H NMR (400 MHz, CDCl₃) δ 8.12 (m, 1H), 7.36 (d, 2H), 7.32 (m, 1H), 7.23 (m, 1H), 7.00 (s, 1H), 6.96 (s, 1H), 6.94 (d, 2H), 6.93 (m, 1H), 6.65 (s, 1H), 4.30 (m, 1H), 4.11 (m, 1H), 3.98 (m, 1H), 3.21 (m, 1H), 1.91 (m, 1H), 1.81 (m, 1H), 1.30 (s, 9H).

### EXAMPLE 29

### 9-(4-tert-Butylphenyl)-N-(2,4-dichlorophenyl)-6,7-dihydro-5H-imidazo[1,2-a][1,4]diazepine-8(9H)-carboxamide

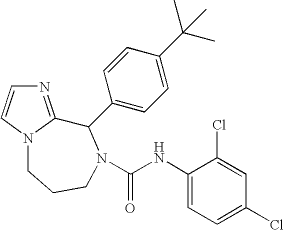

The compound 9-(4-tert-butylphenyl)-N-(2,4-dichlorophenyl)-6,7-dihydro-5H-imidazo[1,2-a][1,4]diazepine-8(9H)-carboxamide was prepared following the procedures described for Example 2 using 2,4-dichlorophenylisocyanate. ¹H NMR (400 MHz, CDCl₃) δ 8.03 (d, 1H), 7.41 (d, 2H), 7.30 (d, 1H), 7.27 (d, 1H), 7.19 (dd, 1H), 7.07 (d, 2H), 7.01 (d, 1H), 6.92 (d, 1H), 6.50 (s, 1H), 4.33 (m, 1H), 4.13 (m, 1H), 4.00 (m, 1H), 3.21 (m, 1H), 2.06 (m, 1H), 1.85 (m, 1H), 1.32 (s, 9H).

### EXAMPLE 30

### 9-(4-tert-Butylphenyl)-6,7,8,9-tetrahydro-5H-imidazo[1,5-a][1,4]diazepine

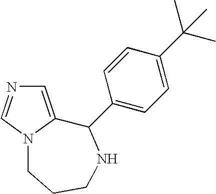

### Step 1

1H-Imidazole (10 g, 147 mmol) was dissolved in THF (150 mL). Dimethylsulfamoyl chloride (19 g, 132 mmol) was added followed by the dropwise addition of triethylamine (20 g, 198 mmol). The mixture was stirred at room temperature overnight. The mixture was poured into H₂O (200 mL) and extracted with EtOAc three times. The combined organic layers were dried over MgSO₄, filtered, and concentrated in vacuo to give crude N,N-dimethyl-1H-imidazole-1-sulfonamide (27 g) that was used without further purification.

### Step 2

N,N-Dimethyl-1H-imidazole-1-sulfonamide (6.26 g, 35.7 mmol) was dissolved in dry THF (100 mL) under N₂ and cooled to −78° C. n-BuLi (16 mL, 42.1 mmol) was added and the mixture was stirred cold for 30 minutes. tert-Butyldimethylsilyl chloride (6.36 g, 42.2 mmol) in THF (50 mL) was added dropwise at −78° C. The mixture was warmed to room temperature for 3 hours. The mixture was cooled to −78° C. and n-BuLi (16 mL, 42.1 mmol) was added dropwise. After one hour, 4-tert-butylbenozylchloride (8.7 g, 44.2 mmol) in THF (50 mL) was added dropwise. The mixture was warmed to room temperature and stirred overnight. Na₂CO₃ (sat. aq., 5 mL) was added. The mixture was washed with CH₂Cl₂. The combined organic layers were washed with brine, dried over Na₂SO₄, and concentrated in vacuo. The crude material was purified by silica gel chromatography to give 5-(4-tert-butylbenozyl)-2-(tert-butyldimethylsilyl)-N,N-dimethyl-1H-imidazole-1-sulfonamide (0.56 g, 3.5%).

### Step 3

HCl (1.5 N, 50 mL) was added to 5-(4-tert-butylbenozyl)-2-(tert-butyldimethylsilyl)-N,N-dimethyl-1H-imidazole-1-sulfonamide (560 mg, 1.25 mmol). The mixture was brought to reflux for one hour. Aqueous NH₃ was added to bring the pH to 8-9 and the mixture was extracted three times with CH₂Cl₂. The combined organic layers were washed with brine, dried over MgSO₄, filtered, and concentrated in vacuo to give (4-tert-butylphenyl)(1H-imidazole-5-yl)methanone. The crude material was used without further purification.

### Step 4

(4-tert-Butylphenyl)(1H-imidazole-5-yl)methanone was dissolved in DMF (30 mL) and NaH (120 mg, 5.0 mmol) was added in portions. tert-Butyl 3-bromopropylcarbamate (700 mg, 2.94 mmol) was added dropwise. The mixture was stirred at room temperature overnight. H₂O (30 mL) was added and the mixture was extracted with EtOAc three times. The combined organic layers were dried over MgSO₄, filtered, and concentrated in vacuo. The crude material was purified by silica gel to give tert-butyl 3-(5-(4-tert-butylbenzoyl)-1H-imidazol-1-yl)propylcarbamate (0.7 g, 70%).

### Step 5

tert-Butyl 3-(5-(4-tert-butylbenzoyl)-1H-imidazol-1-yl)propylcarbamate (700 mg, 1.82 mmol) was dissolved in methanol (60 mL). HCl gas was bubbled through the mixture. The mixture was stirred at room temperature overnight. The mixture was then concentrated, the crude taken up in EtOAc and poured into 3 N NaOH. The mixture was extracted three times with EtOAc. The combined organic layers were dried over MgSO₄, filtered, and concentrated in vacuo to give (1-(3-aminopropyl)-1H-imidazol-5-yl)(4-tert-butylphenyl)methanone. The crude material was used without further purification.

### Step 6

(1-(3-Aminopropyl)-1H-imidazol-5-yl)(4-tert-butylphenyl)methanone (1.3 g, 4.56 mmol), 4-methylbenzenesulfonic acid (100 mg, 0.58 mmol) and toluene (70 mL) were heated to reflux under Dean Stark conditions for 38 hours. The mixture was concentrated and the residue taken up in EtOAc and poured into NaHCO₃. The aqueous phase was extracted with EtOAc three times and the combined organic layers were dried over MgSO₄, filtered, and concentrated in vacuo. The crude material was purified by silica gel chromatography to give (Z)-9-(4-tert-butylphenyl)-6,7-dihydro-5H-imidazo[1,5-a][1,4]diazepine (0.15 g, 12.5%).

### Step 7

(Z)-9-(4-tert-Butylphenyl)-6,7-dihydro-5H-imidazo[1,5-a][1,4]diazepine (150 mg, 0.56 mmol) was dissolved in methanol (30 mL) and NaBH₄ (80 mg, 2.11 mmol) was added in portions at −20° C. The mixture was stirred one hour at −20° C. then H₂O (30 mL) was added. The mixture was extracted three times with EtOAc and the combined organic layers were dried over MgSO₄, filtered, and concentrated in vacuo to give 9-(4-tert-butylphenyl)-6,7,8,9-tetrahydro-5H-imidazo[1,5-a][1,4]diazepine (108 mg, 71%). ¹H NMR (300 MHz, CDCl₃) δ 7.44 (s, 1H), 7.39 (d, 2H), 7.28 (d, 2H), 6.31 (s, 1H), 4.87 (s, 1H), 4.23 (dd, 1H), 4.08 (m, 1H), 3.46 (m, 1H), 3.07 (m, 1H), 1.89 (m, 2H) 1.33 (s, 9H).

### EXAMPLE 31

### 9-(4-tert-Butylphenyl)-N-(2,4-dichlorophenyl)-6,7-dihydro-5H-imidazo[1,5-a][1,4]diazepine-8(9H)-carboxamide

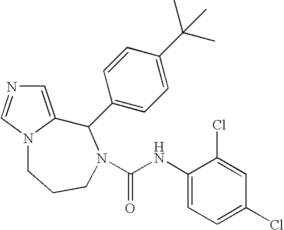

The compound 9-(4-tert-butylphenyl)-N-(2,4-dichlorophenyl)-6,7-dihydro-5H-imidazo[1,5-a][1,4]diazepine-8(9H)-carboxamide was prepared following the procedures described for Example 2 using 9-(4-tert-butylphenyl)-6,7,8,9-tetrahydro-5H-imidazo[1,5-a][1,4]diazepine and 2,4-dichlorophenylisocyanate. ¹H NMR (400 MHz, CDCl₃) δ 8.11 (d, 1H), 7.53 (s, 1H), 7.44 (d, 2H), 7.27 (d, 1H), 7.22 (d, 2H), 7.19 (dd, 1H), 6.98 (s, 1H), 6.64 (b, 1H), 6.32 (b, 1H), 4.16 (m, 3H), 3.18 (m, 1H), 2.21 (m, 2H), 1.34 (s, 9H).

### EXAMPLE 32

### 1-(4-tert-Butylcyclohexyl)-2,3,4,5-tetrahydro-1H-pyrrolo[1,2-a][1,4]diazepine

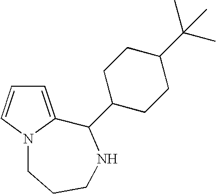

The compound 1-(4-tert-butylcyclohexyl)-2,3,4,5-tetrahydro-1H-pyrrolo[1,2-a][1,4]diazepine was prepared following the procedures described for Example 1 using 4-tert-butylcyclohexanecarbonyl chloride. ¹H NMR (300 MHz, CD₃OD) δ 6.76 (s, 1H), 6.23 (s, 1H), 6.01 (s, 1H), 4.28 (m, 3H), 3.42 (m, 2H), 2.00 (m, 6H), 1.31 (m, 6H), 0.90 (s, 9H).

### EXAMPLE 33

### 1-(4-tert-Butylcyclohexyl)-N-(3,4-difluorophenyl)-4,5-dihydro-1H-pyrrolo[1,2-a][1,4]diazepine-2(3H)-carboxamide

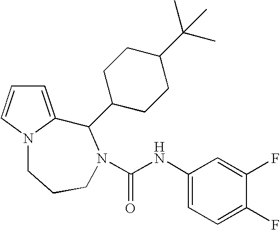

The compound 1-(4-tert-butylcyclohexyl)-N-(3,4-difluorophenyl)-4,5-dihydro-1H-pyrrolo[1,2-a][1,4]diazepine-2(3H)-carboxamide was prepared following the procedures described for Example 2 using 1-(4-tert-butylcyclohexyl)-2,3,4,5-tetrahydro-1H-pyrrolo[1,2-a][1,4]diazepine and 3,4-difluorophenylisocyanate. ¹H NMR (400 MHz, CDCl₃) δ 7.28 (m, 1H), 7.01 (q, 1H), 6.80 (m, 1H), 6.59 (s, 1H), 6.54 (s, 1H), 6.06 (s, 1H), 5.98 (s, 1H), 4.50 (m, 1H), 4.33 (m, 1H), 4.17 (m, 2H), 3.21 (m, 1H), 2.01 (m, 2H), 1.84 (m, 4H), 1.51 (m, 1H), 0.98 (m, 5H), 0.84 (s, 9H).

### EXAMPLE 34

### 1-(4-tert-Butylcyclohexyl)-N-(2,4-difluorophenyl)-4,5-dihydro-1H-pyrrolo[1,2-a][1,4]diazepine-2(3H)-carboxamide

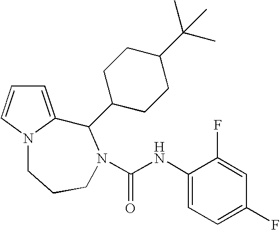

The compound 1-(4-tert-butylcyclohexyl)-N-(2,4-difluorophenyl)-4,5-dihydro-1H-pyrrolo[1,2-a][1,4]diazepine-2(3H)-carboxamide was prepared following the procedures described for Example 2 using 2,4-difluorophenylisocyanate. ¹H NMR (400 MHz, CDCl₃) δ 7.95 (m, 1H), 6.81 (m, 2H), 6.73 (s, 1H), 6.53 (s, 1H), 6.08 (s, 1H), 5.97 (s, 1H), 4.45 (m, 2H), 4.15 (m, 2H), 3.22 (m, 1H), 2.01 (m, 2H), 1.82 (m, 4H), 1.51 (m, 1H), 0.96 (m, 5H), 0.84 (s, 9H).

### EXAMPLE 35

### 1-(4-tert-Butylcyclohexyl)-N-(2,4-dichlorophenyl)-4,5-dihydro-1H-pyrrolo[1,2-a][1,4]diazepine-2(3H)-carboxamide

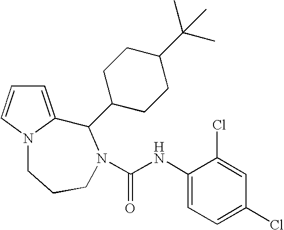

The compound 1-(4-tert-butylcyclohexyl)-N-(2,4-dichlorophenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.08 (d, 1H), 7.31 (d, 1H), 7.18 (dd, 1H), 7.09 (s, 1H), 6.53 (s, 1H), 6.11 (s, 1H), 5.94 (m, 1H), 4.49 (m, 2H), 4.15 (m, 2H), 3.23 (m, 1H), 1.93 (m, 4H), 1.85 (m, 1H), 1.78 (m, 1H), 1.52 (m, 1H), 0.98 (m, 5H), 0.84 (s, 9H).

### EXAMPLE 36

### 4-(4-tert-Butylphenyl)-N-(2,4-difluorophenyl)-7,8-dihydro-4H-pyrazolo[1,5-a][1,4]diazepine-5(6H)-carboxamide

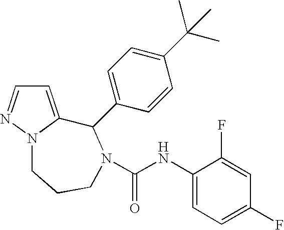

The compound 4-(4-tert-butylphenyl)-N-(2,4-difluorophenyl)-7,8-dihydro-4H-pyrazolo[1,5-a][1,4]diazepine-5(6H)-carboxamide was prepared following the procedures described for Example 2 using 2,4-difluorophenylisocyanate. ¹H NMR (400 MHz, DMSO) δ 8.02 (m, 1 H), 7.42 (m, 3 H), 7.13 (d, 2 H, J=8.0 Hz), 6.77-6.86 (m, 2 H), 6.55 (m,2 H), 6.00 (s, 1 H), 4.48 (m, 1 H), 4.36 (m, 1 H), 4.08 (m, 1 H), 3.20 (m, 1 H0, 2.15 (m,1 H), 1.90 (m, 1 H), 1.29 (s, 9 H).

### EXAMPLE 37

### 9-(4-tert-Butylphenyl)-N-(2,4-difluorophenyl)-6,7-dihydro-5H-imidazo[1,5-a][1,4]diazepine-8(9H)-carboxamide

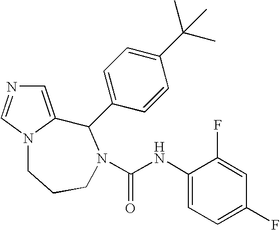

The compound 9-(4-tert-butylphenyl)-N-(2,4-difluorophenyl)-6,7-dihydro-5H-imidazo[1,5-a][1,4]diazepine-8(9H)-carboxamide was prepared following the procedures for Example 2 using 9-(4-tert-butylphenyl)-6,7,8,9-tetrahydro-5H-imidazo[1,5-a][1,4]diazepine and 2,4-difluorophenylisocyanate. ¹H NMR (400 MHz, DMSO) δ 8.00 (m, 1 H), 7.51 (s, 1 H), 7.43 (m, 2 H), 7.19 (d, 2 H, J=8.4 Hz), 6.70-6.85(m, 2 H), 6.67 (s, 1 H), 6.57 (d, 1 H, J=3.2 Hz), 6.32 (b, 1 H), 4.06-4.30 (m, 3 H), 3.20 (m, 1 H), 2.16 (m, 1 H), 1.86 (m, 1 H), 1.34 (s, 9 H).

### EXAMPLE 38

### 1-(4-(Trifluoromethyl)phenyl)-2,3,4,5-tetrahydro-1H-pyrrolo[1,2-a][1,4]diazepine

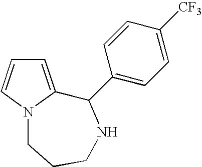

The compound 1-(4-(trifluoromethyl)phenyl)-2,3,4,5-tetrahydro-1H-pyrrolo[1,2-a][1,4]diazepine was prepared following the procedures described for Example 1 using 4-trifluoromethylbenzoyl chloride. ¹H NMR (300 MHz, CDCl₃) δ 7.61 (d, 2H), 7.49 (d, 2H), 6.60 (s, 1H), 5.90 (t, 1H), 5.33 (s, 1H), 4.98 (s, 1H), 4.14 (m, 2H), 3.42 (m, 1H), 3.08 (m, 1H), 1.87 (m, 3H).

### EXAMPLE 39

### N-(2,4-Difluorophenyl)-1-(4-(trifluoromethyl)phenyl)-4,5-dihydro-1H-pyrrolo[1,2-a][1,4]diazepine-2(3H)-carboxamide

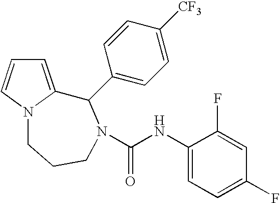

The compound N-(2,4-difluorophenyl)-1-(4-(trifluoromethyl)phenyl)-4,5-dihydro-1H-pyrrolo[1,2-a][1,4]diazepine-2(3H)-carboxamide was prepared following the procedures described for Example 2 using 1-(4-(trifluoromethyl)phenyl)-2,3,4,5-tetrahydro-1H-pyrrolo[1,2-a][1,4]diazepine and 2,4-difluorophenylisocyanate. ¹H NMR (400 MHz, CDCl₃) δ 8.01 (dt, 1H), 7.67 (d, 2H), 7.42 (d, 2H), 6.80 (m, 2H), 6.69 (dd, 1H), 6.56 (d, 1H), 6.34 (b, 1H), 6.05 (dd, 1H), 5.69 (b, 1H), 4.16 (m, 1H), 4.12 (m, 1H), 4.00 (m, 1H), 3.10 (t, 1H), 2.13 (m, 1H), 1.87 (m, 1H).

### EXAMPLE 40

### N-(2,4-Difluorophenyl)-1-(4-(trifluoromethoxy)phenyl)-4,5-dihydro-1H-pyrrolo[1,2-a][1,4]diazepine-2(3H)-carboxamide

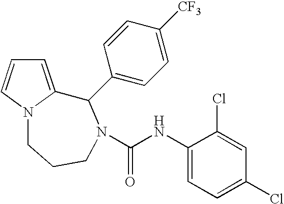

The compound N-(2,4-difluorophenyl)-1-(4-(trifluoromethoxy)phenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.14 (d, 1H), 7.68 (d, 2H), 7.44 (d, 2H), 7.28 (d, 1H), 7.19 (dd, 1H), 6.95 (s, 1H), 6.69 (dd, 1H), 6.36 (b, 1H), 6.04 (dd, 1H), 5.64 (b, 1H), 4.18 (m, 1H), 4.13 (m, 1H), 4.00 (m, 1H), 3.10 (t, 1H), 2.19 (m, 1H), 1.85 (m, 1H).

### EXAMPLE 41

### 1-Benzyl-N-(2,4-difluorophenyl)-4,5-dihydro-1H-pyrrolo[1,2-a][1,4]diazepine-2(3H)-carboxamide

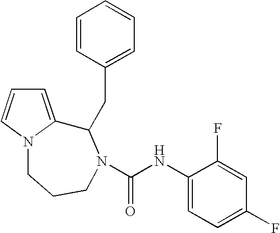

The compound 1-benzyl-N-(2,4-difluorophenyl)-4,5-dihydro-1H-pyrrolo[1,2-a][1,4]diazepine-2(3H)-carboxamide was prepared following the procedures described for Example 2 using 2,4-difluorophenylisocyanate. ¹H NMR (300 MHz, CDCl₃) δ 7.72 (m, 1H), 7.30 (m, 5H), 6.77 (m, 2H), 6.80 (b, 1H), 6.59 (m, 1H), 6.19 (b, 1H), 6.05 (dd, 1H), 4.11 (m, 3H), 3.41 (dd, 1H), 3.25 (m, 1H), 2.10 (m, 1H), 1.86 (m, 1H).

### EXAMPLE 42

### 1-(4-Chlorophenyl)-2,3,4,5-tetrahydro-1H-pyrrolo[1,2-a][1,4]diazepine

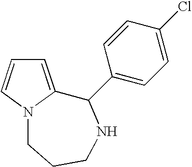

The compound 1-(4-chlorophenyl)-2,3,4,5-tetrahydro-1H-pyrrolo[1,2-a][1,4]diazepine was prepared following the procedures described for Example 1 using 4-chlorobenzoyl chloride. ¹H NMR (400 MHz, CDCl₃) δ 7.31 (m, 4H), 6.59 (s, 1H), 5.89 (t, 1H), 5.35 (s, 1H), 4.88 (s, 1H), 4.12 (m, 2H), 3.39 (m, 1H), 3.06 (m, 1H), 1.90 (m, 3H).

### EXAMPLE 43

### 1-(4-Chlorophenyl)-N-(2,4-difluorophenyl)-4,5-dihydro-1H-pyrrolo[1,2-a][1,4]diazepine-2(3H)-carboxamide

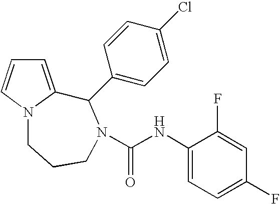

The compound 1-(4-chlorophenyl)-N-(2,4-difluorophenyl)-4,5-dihydro-1H-pyrrolo[1,2-a][1,4]diazepine-2(3H)-carboxamide was prepared following the procedures described for Example 2 using 1-(4-chlorophenyl)-2,3,4,5-tetrahydro-1H-pyrrolo[1,2-a][1,4]diazepine and 2,4-difluorophenylisocyanate. ¹H NMR (400 MHz, CDCl₃) δ 8.21 (dt, 1H), 7.39 (d, 2H), 7.24 (d, 2H), 6.80 (m, 2H), 6.67 (dd, 1H), 6.56 (d, 1H), 6.20 (b, 1H), 6.03 (dd, 1H), 5.66 (b, 1H), 4.19 (m, 1H), 4.08 (m, 1H), 3.96 (m, 1H), 3.05 (b, 1H), 2.13 (m, 1H), 1.81 (m, 1H).

### EXAMPLE 44

### 1-(4-Chlorophenyl)-N-(2,4-dichlorophenyl)-4,5-dihydro-1H-pyrrolo[1,2-a][1,4]diazepine-2(3H)-carboxamide

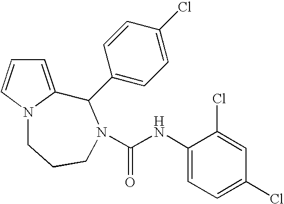

The compound 1-(4-chlorophenyl)-N-(2,4-dichlorophenyl)-4,5-dihydro-1H-pyrrolo[1,2-a][1,4]diazepine-2(3H)-carboxamide was prepared following the procedures described for Example 2 using 2,4-dichlorophenylisocyanate. ¹H NMR (400 MHz, CDCl₃) δ 8.15 (d, 1H), 7.39 (d, 2H), 7.28 (d, 1H), 7.25 (d, 2H), 7.19 (dd, 1H), 6.96 (s, 1H), 6.67 (dd, 1H), 6.23 (b, 1H), 6.03 (dd, 1H), 5.62 (b, 1H), 4.20 (m, 1H), 4.09 (m, 1H), 4.00 (m, 1H), 3.05(b, 1H), 2.17 (m, 1H), 1.85 (m, 1H).

### EXAMPLE 45

### (1-(4-tert-Butylcyclohexyl)-4,5-dihydro-1H-pyrrolo[1,2-a][1,4]diazepin-2(3H)-yl)(2,4-difluorophenyl)methanone

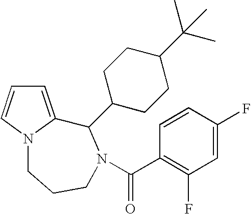

A mixture of 1-(4-tert-butylcyclohexyl)-2,3,4,5-tetrahydro- 1H-pyrrolo[1,2-a][1,4]diazepine (32 mg, 0.12 mmol), 2,4-difluorobenzoyl chloride (25 mg, 0.14 mmol) and triethylamine (21 μL, 0.18 mmol) in THF (2.5 mL) was stirred for 2 hours. The mixture was diluted with ethyl acetate, washed with water, brine, dried over Na₂SO₄ and concentrated in vacuo. The crude material was purified by reverse phase HPLC to give 22.8 mg (46% yield) of the title compound. ¹H NMR (400 MHz, CDCl₃) δ 8.04 (m, 1H), 6.89 (m, 2H), 6.51(s, 1H), 5.88 (m, 1H), 5.47 (s, 1H), 4.98 (d, 1H), 4.14 (m, 3H), 3.11 (t, 2H), 1.87(m, 8H), 1.26 (d, 1H), 1.00 (m, 2H), 0.81 (s, 9H). MS (M/z, M+1): 415.

### EXAMPLE 46

### 1-(1-(4-tert-Butylphenyl)-4,5-dihydro-1H-pyrrolo[1,2-a][1,4]diazepin-2(3H)-yl)ethanone

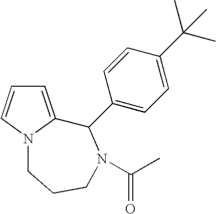

The compound 1-(1-(4-tert-butylphenyl)-4,5-dihydro-1H-pyrrolo[1,2-a][1,4]diazepin-2(3H)-yl)ethanone was prepared following the procedures described for Example 45 using acetyl chloride. ¹H NMR (400 MHz, CDCl₃) δ 7.36 (d, 2H), 7.06 (s, 1H), 6.96 (d, 2H), 6.63 (t, 1H), 6.16 (s, 1H), 6.04 (s, 1H), 4.61 (m. 1H), 3.95 (m, 2H), 3.37 (m, 1H), 2.25 (s, 3H), 1.86 (m, 2H), 1.31 (s, 9H).

### EXAMPLE 47

### (9-(4-tert-Butylphenyl)-6,7-dihydro-5H-imidazo[1,2-a][1,4]diazepin-8(9H)-yl)(2,4-difluorophenyl)methanone

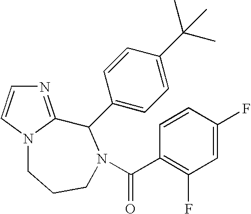

The compound (9-(4-tert-butylphenyl)-6,7-dihydro-5H-imidazo[1,2-a][1,4]diazepin-8(9H)-yl)(2,4-difluorophenyl)methanone was prepared following the procedures for Example 45 using 2,4-difluorobenzoyl chloride. ¹H NMR (400 MHz, CDCl₃) δ 7.86 (q, 1H), 7.36 (d, 2H), 7.04 (d, 1H), 6.92 (d, 1H), 6.88 (d, 2H), 6.83 (m, 2H), 6.40 (s, 1H), 4.83 (m, 1H), 4.13 (m, 1H), 3.91 (m, 1H), 3.12 (m, 1H), 2.00 (m, 2H), 1.30 (s, 9H).

### EXAMPLE 48

### (9-(4-tert-Butylphenyl)-6,7-dihydro-5H-imidazo[1,2-a][1,4]diazepin-8(9H)-yl)(2,4-dichlorophenyl)methanone

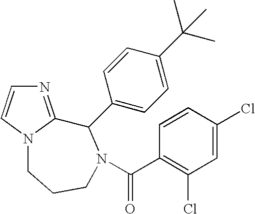

The compound (9-(4-tert-butylphenyl)-6,7-dihydro-5H-imidazo[1,2-a][1,4]diazepin-8(9H)-yl)(2,4-dichlorophenyl)methanone was prepared following the procedures described for Example 45 using 2,4-dichlorobenzoyl chloride. ¹H NMR (400 MHz, CDCl₃) δ 7.73 (d, 1H), 7.40 (m, 1H), 7.35 (d, 2H), 7.26 (m, 1H), 7.08 (d, 1H), 6.94 (d, 1H), 6.90 (d, 2H), 6.88 (m, 1H), 6.27 (s, 1H), 4.89 (m, 1H), 4.15 (m, 1H), 3.94 (m, 2H), 3.12 (m, 1H), 2.00 (m, 2H), 1.29 (s, 9H).

### EXAMPLE 49

### (1-(4-tert-Butylphenyl)-4,5-dihydro-1H-pyrrolo[1,2-a][1,4]diazepin-2(3H)-yl)(2,4-dichlorophenyl)methanone

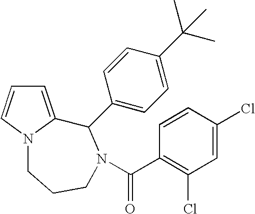

The compound (1-(4-tert-butylphenyl)-4,5-dihydro-1H-pyrrolo[1,2-a][1,4]diazepin-2(3H)-yl)(2,4-dichlorophenyl)methanone was prepared following the procedures described for Example 45 using 2,4-dichlorobenzoyl chloride. LCMS: Calc. MW=441.39; Found (M/z, M+1): 442.25.

### EXAMPLE 50

### 1-(1-(4-tert-Butylphenyl)-4,5-dihydro-1H-pyrrolo[1,2-a][1,4]diazepin-2(3H)-yl)-2-(4-fluorophenyl)ethanone

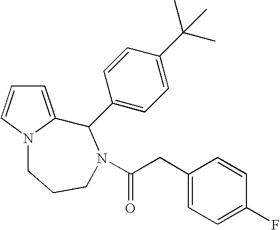

The compound 1-(1-(4-tert-butylphenyl)-4,5-dihydro-1H-pyrrolo[1,2-a][1,4]diazepin-2(3H)-yl)-2-(4-fluorophenyl)ethanone was prepared following the procedures described for Example 45 using 2-(4-fluorophenyl)acetyl chloride. LCMS: Calc. MW=404.52; Found (M/z, M+1): 405.27.

### EXAMPLE 51

### 1-(1-(4-tert-Butylphenyl)-4,5-dihydro-1H-pyrrolo[1,2-a][1,4]diazepin-2(3H)-yl)-2-(2,6-difluorophenyl)ethanone

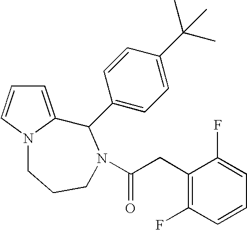

The compound 1-(1-(4-tert-butylphenyl)-4,5-dihydro-1H-pyrrolo[1,2-a][1,4]diazepin-2(3H)-yl)-2-(2,6-difluorophenyl)ethanone was prepared following the procedures described for Example 45 using 2′-(2,6-difluorophenyl)acetyl chloride. LCMS: Calc. MW=422.51; Found (M/z, M+1): 423.23.

### EXAMPLE 52

### 1-(1-(4-tert-Butylphenyl)-4,5-dihydro-1H-pyrrolo[1,2-a][1,4]diazepin-2(3H)-yl)-2-morpholinoethanone

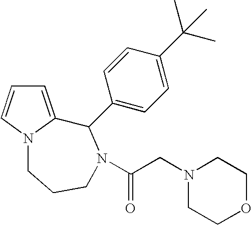

The compound 1-(1-(4-tert-butylphenyl)-4,5-dihydro-1H-pyrrolo[1,2-a][1,4]diazepin-2(3H)-yl)-2-morpholinoethanone was prepared following the procedures described for Example 45 using 2-morpholinoacetyl chloride. ¹H NMR (400 MHz, CDCl₃) δ 7.35 (m, 3H), 7.09 (d, 1H), 6.87 (m, 4H), 6.40 (d, 1H), 4.89 (m, 1H), 4.13 (m, 1H), 3.94 (m, 2H), 3.12 (m, 1H), 2.00 (m, 2H), 1.29 (s, 9H).

### EXAMPLE 53

### 2-(1-(4-tert-Butylphenyl)-4,5-dihydro-1H-pyrrolo[1,2-a][1,4]diazepin-2(3H)-yl)benzo[d]oxazole

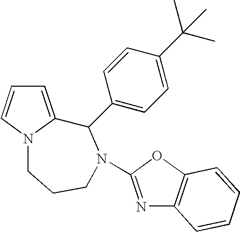

A mixture of 1-(4-tert-butylphenyl)-2,3,4,5-tetrahydro-1H-pyrrolo[1,2-a][1,4]diazepine (136 mg, 0.51 mmol), 2-chlorobenzo[d]oxazole (60 μL, 0.51 mmol) and diisopropylethylamine (175 μL, 1.0 mmol) in toluene (3 mL) was sealed in a high pressure tube. The mixture was heated to 150° C. in a microwave reactor for 2 hours. The mixture was diluted with ethyl acetate (20 mL) and washed with water, brine, dried over Na₂SO₄ and concentrated in vacuo. The crude material was purified by silica gel chromatography to give the title compound (23 mg, 11% yield). ¹H NMR (400 MHz, DMSO) δ 7.40 (m, 3H), 7.28 (d, 1H), 7.13 (t, 1H), 7.00 (m, 3H), 6.77 (s, 1H), 6.62 (s, 1H), 5.89 (m, 2H), 4.22 (m, 1H), 4.11 (m, 1H), 3.80 (m, 1H), 3.17 (m, 1H), 1.80 (m, 2H), 1.26 (s, 9H).

### EXAMPLE 54

### 2-(6-(4-tert-Butylbenzyl)-3,4-dihydropyrrolo[1,2-a]pyrimidin-1(2H)-yl)benzo[d]oxazole

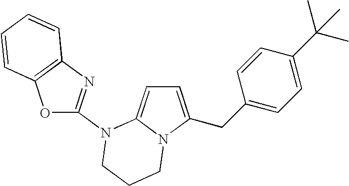

The compound 2-(6-(4-tert-butylbenzyl)-3,4-dihydropyrrolo[1,2-a]pyrimidin-1(2H)-yl)benzo[d]oxazole was isolated by silica gel chromatography from the procedure described for Example 53 to give 25.2 mg (12% yield). ¹H NMR (400 MHz, DMSO) δ 7.49 (d, 1H), 7.38 (d, 1H), 7.29 (d, 2H), 7.19 (t, 1H), 7.10 (d, 2H), 7.07 (t, 1H), 6.31 (d, 1H), 5.75 (d, 1H), 4.01 (t, 2H), 3.85 (s, 2H), 3.77 (t, 2H), 2.10 (m, 2H), 1.24 (s, 9H).

### EXAMPLE 55

### 8-(4-tert-Butylphenyl)-5,6-dihydroindolizine-7-carboxylic acid

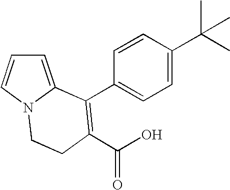

### Step 1

A mixture of (4-tert-butylphenyl)(1H-pyrrol-2-yl)methanone (2.7 g, 11.9 mmol), methyl 4-bromobutanoate (1.65 mL, 11.9 mmol) and Cs₂CO₃ (5.8 g, 17.8 mmol) in DMF (50 mL) was heated to 150° C. The mixture was stirred at 150° C. for 12 hours then diluted with water. The mixture was cooled to room temperature and extracted with ethyl acetate. The organic layers were washed with brine, dried over Na₂SO₄, and concentrated in vacuo. The crude material was purified by silica gel chromatography to give 3.07 g (79% yield) of methyl 4-(2-(4-tert-butylbenzoyl)-1H-pyrrol-1-yl)butanoate. ¹H NMR (400 MHz, CDCl₃) δ 7.74 (d, 2H), 7.45 (d, 2H), 6.95 (dd, 1H), 6.76 (dd, 1H), 6.14 (dd, 1H), 4.45 (t, 2H), 3.64 (s, 3H), 2.33 (t, 2H), 2.15 (m, 2H), 1.34 (s, 9H).

### Step 2

A solution of methyl 4-(2-(4-tert-butylbenzoyl)-1H-pyrrol-1-yl)butanoate (2.24 g, 6.86 mmol) in THF (100 mL) was cooled to −78° C. and LiHMDS/THF (1.0 M, 7.54 mL) was added. The mixture was stirred at −78° C. for 30 minutes then warmed to room temperature and stirred for 1 hour. The mixture was cooled to −78° C.; mesyl chloride (0.58 mL, 7.54 mmol) was added and the mixture warmed to room temperature and stirred for 1 hour. The mixture was diluted with ethyl acetate, washed with water, brine, dried over Na₂SO₄ and concentrated in vacuo. The crude material was suspended in toluene (100 mL) and p-toluene-sulfonyl chloride (50 mg) was added. The mixture was refluxed for 3 hours. The mixture was concentrated in vacuo and the residue was purified by silica gel chromatography to give 1.4 g (66% yield) of methyl 8-(4-tert-butylphenyl)-5,6-dihydroindolizine-7-carboxylate. ¹H NMR (400 MHz, CDCl₃) δ 7.37 (d, 2H), 7.15 (d, 2H), 6.76 (dd, 1H), 6.12 (dd, 1H), 5.88 (dd, 1H), 4.08 (t, 2H), 3.48 (s, 3H), 2.96 (t, 2H), 1.34 (s, 9H).

### Step 3

To a solution of methyl 8-(4-tert-butylphenyl)-5,6-dihydroindolizine-7-carboxylate (223 mg, 0.72 mmol) in THF/MeOH (3:1, 10 mL) was added 1 N LiOH (2.5 mL, 2.5 mmol). The mixture was heated to 65° C. and stirred for 3 hours. The mixture was concentrated in vacuo. To the aqueous mixture was added 1 N HCl (2.5 mL) and the mixture extracted with ethyl acetate. The organic layer was washed with brine, dried over Na₂SO₄ and concentrated in vacuo to give 220 mg (99% yield) of the title compound. ¹H NMR (400 MHz, CDCl₃) δ 7.35 (d, 2H), 7.16 (d, 2H), 6.77 (dd, 1H), 6.12 (dd, 1H), 5.85 (dd, 1H), 4.07 (t, 2H), 2.95 (t, 2H), 1.35 (s, 9H).

### EXAMPLE 56

### 8-(4-tert-Butylphenyl)-N-(2,4-difluorophenyl)-5,6-dihydroindolizine-7-carboxamide

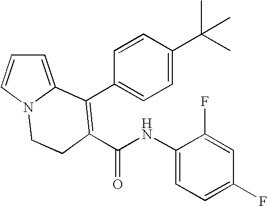

To a solution of 8-(4-tert-butylphenyl)-5,6-dihydroindolizine-7-carboxylic acid (82 mg, 0.28 mmol) in CH₃CN (10 mL) was added 1-(3-dimethylaminopropyl)-3-ethylcarbodiimide hydrochloride (64 mg, 0.33 mmol), benzotriaz-1-ol (56 mg, 0.42 mmol) and triethylamine (77 μL, 0.55 mmol). The mixture was stirred for 10 minutes and then 2,4-difluoroaniline was added. The mixture was stirred overnight at 65° C. Ethyl acetate was added and the mixture was washed with water, brine, dried over Na₂SO₄ and concentrated in vacuo. The crude material was purified by prep TLC to give the title compound (22 mg, 5% yield). ¹H NMR (400 MHz, CDCl₃) δ 7.98 (d, 1H), 7.44 (m, 1H), 7.41 (d, 2H), 7.37 (m, 1H), 7.32 (d, 2H), 7.19 (d, 1H), 6.92 (dd, 1H), 6.22 (dd, 1H), 6.06 (dd, 1H), 4.24 (t, 2H), 3.21 (t, 2H), 1.29 (s, 9H).

### EXAMPLE 57

### 8-(4-tert-Butylphenyl)-N-(2,4-dichlorophenyl)-5,6-dihydroindolizine-7-carboxamide

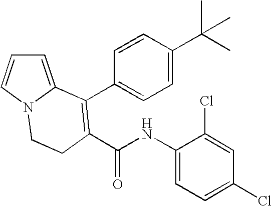

The compound 8-(4-tert-butylphenyl)-N-(2,4-dichlorophenyl)-5,6-dihydroindolizine-7-carboxamide was prepared following the procedures described for Example 56 using 2,4-dichloroaniline. ¹H NMR (400 MHz, CDCl₃) δ 7.98 (d, 1H), 7.44 (m, 1H), 7.41 (d, 2H), 7.37 (m, 1H), 7.33 (d, 2H), 7.19 (d, 1H), 6.92 (dd, 1H), 6.22 (dd, 1H), 6.06 (dd, 1H), 4.24 (t, 2H), 3.21 (t, 2H), 1.29 (s, 9H).

### EXAMPLE 58

### 8-(4-tert-Butylphenyl)-N-(4-(trifluoromethoxy)phenyl)-5,6-dihydroindolizine-7-carboxamide

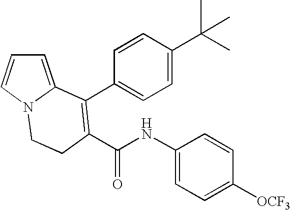

The compound 8-(4-tert-butylphenyl)-N-(4-(trifluoromethoxy)phenyl)-5,6-dihydroindolizine-7-carboxamide was prepared following the procedures described for Example 56 using 4-trifluoromethoxyaniline. ¹H NMR (400 MHz, CDCl₃) δ 7.52 (d, 2H), 7.35 (d, 2H), 6.97 (d, 2H), 6.88 (d, 2H), 6.82 (s, 1H), 6.80 (dd, 1H), 6.17 (dd, 1H), 5.91 (dd, 1H), 4.13 (t, 2H), 3.08 (t, 2H), 1.39 (s, 9H).

### EXAMPLE 59

### 8-(4-tert-Butylphenyl)-N-(3,4-difluorophenyl)-5,6-dihydroindolizine-7-carboxamide

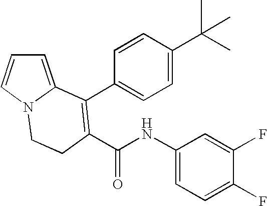

The compound 8-(4-tert-butylphenyl)-N-(3,4-difluorophenyl)-5,6-dihydroindolizine-7-carboxamide was prepared following the procedures described for Example 56 using 3,4-difluoroaniline. ¹H NMR (400 MHz, CDCl₃) δ 7.53 (d, 2H), 7.35 (d, 2H), 6.99 (m, 1H), 6.88 (q, 1H), 6.80 (dd, 1H), 6.75 (s, 1H), 6.36 (m, 1H), 6.16 (dd, 1H), 5.91 (dd, 1H), 4.15 (t, 2H), 3.08 (t, 2H), 1.39 (s, 9H).

### EXAMPLE 60

### 8-(4-tert-Butylphenyl)-N-(4-chlorophenyl)-5,6-dihydroindolizine-7-carboxamide

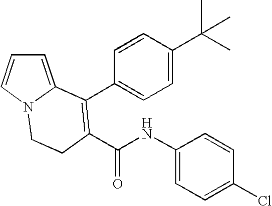

The compound 8-(4-tert-butylphenyl)-N-(4-chlorophenyl)-5,6-dihydroindolizine-7-carboxamide was prepared following the procedures described for Example 56 using 4-chloroaniline. ¹H NMR (400 MHz, CDCl₃) δ 7.51 (d, 2H), 7.35 (d, 2H), 7.08 (d, 2H), 6.81 (d, 1H), 6.80 (dd, 1H), 6.78 (s, 1H), 6.16 (dd, 1H), 5.91 (dd, 1H), 4.13 (t, 2H), 3.07 (t, 2H), 1.39 (s, 9H).

### EXAMPLE 61

### (E)-9-(4-tert-Butylphenyl)-6,7-dihydro-5H-pyrrolo[1,2-a]azepine-8-carboxylic acid

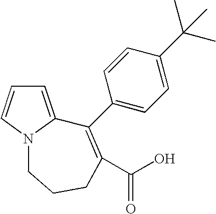

The compound (E)-9-(4-tert-butylphenyl)-6,7-dihydro-5H-pyrrolo[1,2-a]azepine-8-carboxylic acid was prepared following the procedures described for Example 55 using methyl 5-bromopentanoate. ¹H NMR (400 MHz, CDCl₃) δ 7.30 (d, 2H), 7.16 (d, 2H), 6.85 (dd, 1H), 6.10 (dd, 1H), 5.85 (dd, 1H), 4.11 (t, 2H), 2.66 (t, 2H), 2.24 (m, 2H), 1.33 (s, 9H).

### EXAMPLE 62

### (E)-9-(4-tert-Butylphenyl)-N-(2,4-difluorophenyl)-6,7-dihydro-5H-pyrrolo[1,2-a]azepine-8-carboxamide

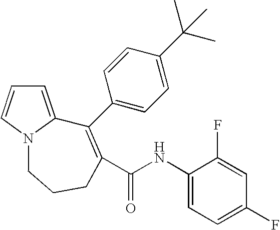

The compound (E)-9-(4-tert-butylphenyl)-N-(2,4-difluorophenyl)-6,7-dihydro-5H-pyrrolo[1,2-a]azepine-8-carboxamide was prepared following the procedures for Example 56 using 2,4-difluoroaniline. ¹H NMR (400 MHz, CDCl₃) δ 7.98 (d, 1H), 7.43 (d, 2H), 7.37 (d, 2H), 7.36 (m, 2H), 6.98 (dd, 1H), 6.92 (d, 1H), 6.17 (dd, 1H), 5.97 (dd, 1H), 4.26 (t, 2H), 2.90 (t, 2H), 2.42 (m, 2H), 1.33 (s, 9H).

### EXAMPLE 63

### (E)-9-(4-tert-Butylphenyl)-N-(2,4-dichlorophenyl)-6,7-dihydro-5H-pyrrolo[1,2-a]azepine-8-carboxamide

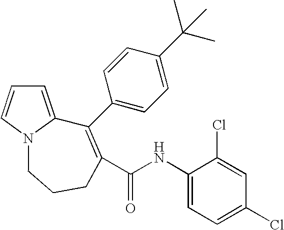

The compound (E)-9-(4-tert-butylphenyl)-N-(2,4-dichlorophenyl)-6,7-dihydro-5H-pyrrolo[1,2-a]azepine-8-carboxamide was prepared following the procedures described for Example 56 using 2,4-dichloroaniline. ¹H NMR (400 MHz, CDCl₃) δ 8.41 (d, 1H), 7.39 (b, 1H), 7.26 (m, 4H), 7.15 (m, 2H), 6.87 (dd, 1H), 6.13 (dd, 1H), 5.92 (dd, 1H), 4.14 (t, 2H), 2.79 (t, 2H), 2.28 (m, 2H), 1.26 (s, 9H).

### EXAMPLE 64

### (E)-9-(4-tert-Butylphenyl)-N-(4-(trifluoromethyl)phenyl)-6,7-dihydro-5H-pyrrolo[1,2-a]azepine-8-carboxamide

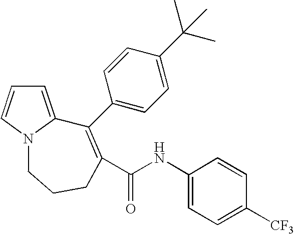

The compound (E)-9-(4-tert-butylphenyl)-N-(4-(trifluoromethyl)phenyl)-6,7-dihydro-5H-pyrrolo[1,2-a]azepine-8-carboxamide was prepared following the procedures described for Example 56 using 4-trifluoromethylaniline. ¹H NMR (400 MHz, CDCl₃) δ 7.38(d, 2H), 7.37(d, 2H), 7.27 (d, 2H), 6.99 (d, 2H), 6.88 (dd, 1H),6.85 (s, 1H), 6.13 (dd, 1H), 5.92 (dd, 1H), 4.14 (t, 2H), 2.82 (t, 2H), 2.29 (m, 2H), 1.30 (s, 9H).

### EXAMPLE 65

### (E)-9-(4-tert-Butylphenyl)-N-(3,4-difluorophenyl)-6,7-dihydro-5H-pyrrolo[1,2-a]azepine-8-carboxamide

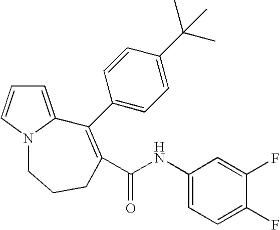

The compound (E)-9-(4-tert-butylphenyl)-N-(3,4-difluorophenyl)-6,7-dihydro-5H-pyrrolo[1,2-a]azepine-8-carboxamide was prepared following the procedures described for Example 56 using 3,4-difluoroaniline. ¹H NMR (400 MHz, CDCl₃) δ 7.38 (d, 2H), 7.27 (d, 2H), 6.95 (m, 1H), 6.88 (dq, 1H), 6.87 (dd, 1H), 6.67 (s, 1H), 6.38 (m, 1H), 6.13 (dd, 1H), 5.91 (dd, 1H), 4.13 (t, 2H), 2.80 (t, 2H), 2.28 (m, 2H), 1.31 (s, 9H).

### EXAMPLE 66

### (E)-9-(4-tert-Butylphenyl)-N-(4-chlorophenyl)-6,7-dihydro-5H-pyrrolo[1,2-a]azepine-8-carboxamide

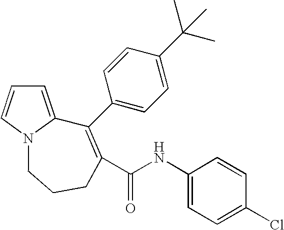

The compound (E)-9-(4-tert-butylphenyl)-N-(4-chlorophenyl)-6,7-dihydro-5H-pyrrolo[1,2-a]azepine-8-carboxamide was prepared following the procedures described for Example 56 using 4-chloroaniline. ¹H NMR (400 MHz, CDCl₃) δ 7.37 (d, 2H), 7.27 (d, 2H), 7.08 (d, 2H), 6.87 (dd, 2H), 6.79 (d, 2H), 6.70 (s, 1H), 6.13 (dd, 1H), 5.91 (dd, 1H), 4.13 (t, 2H), 2.80 (t, 2H), 2.28 (m, 2H), 1.31 (s, 9H).

### EXAMPLE 67

### 8-Phenyl-5,6-dihydroindolizine-7-carboxylic acid

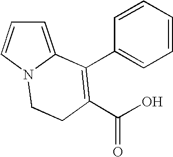

The compound 8-phenyl-5,6-dihydroindolizine-7-carboxylic acid was prepared following the procedures described for Example 55 using benzoyl chloride. 1H NMR (400 MHz, CDCl₃) δ 7.34 (m, 3H), 7.24 (m, 2H), 6.78 (dd, 1H), 6.13 (dd, 1H), 5.82 (dd, 1H), 4.07 (t, 2H), 2.94 (t, 2H).

### EXAMPLE 68

### 8-Phenyl-N-(4-(trifluoromethyl)phenyl)-5,6-dihydroindolizine-7-carboxamide

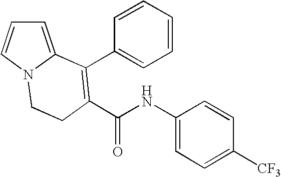

The compound 8-phenyl-N-(4-(trifluoromethyl)phenyl)-5,6-dihydroindolizine-7-carboxamide was prepared following the procedures described for Example 56 using 4-trifluoromethylaniline. ¹H NMR (400 MHz, CDCl₃) δ 7.52 (m, 3H), 7.45 (m, 2H), 7.40 (d, 2H), 7.03 (d, 2H), 6.93 (s, 1H), 6.81 (dd, 1H), 6.17 (dd, 1H), 5.86 (dd, 1H), 4.14 (t, 2H), 3.09 (t, 2H).

### EXAMPLE 69

### 8-Phenyl-N-(4-(trifluoromethyl)phenyl)-5,6-dihydroindolizine-7-carboxamide

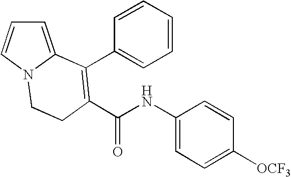

The compound 8-phenyl-N-(4-(trifluoromethyl)phenyl)-5,6-dihydroindolizine-7-carboxamide was prepared following the procedures described for Example 56 using 4-trifluoromethoxyaniline. ¹H NMR (400 MHz, CDCl₃) δ 7.51 (m, 3H), 7.44 (m, 2H), 7.01 (d, 2H), 6.94 (d, 2H), 6.82 (s, 1H), 6.80 (dd, 1H), 6.16 (dd, 1H), 5.85 (dd, 1H), 4.14 (t, 2H), 3.09 (t, 2H).

### EXAMPLE 70

### N-(2,4-Difluorophenyl)-8-phenyl-5,6-dihydroindolizine-7-carboxamide

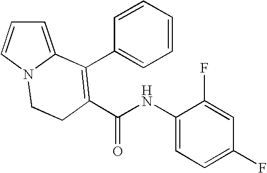

The compound N-(2,4-difluorophenyl)-8-phenyl-5,6-dihydroindolizine-7-carboxamide was prepared following the procedures described for Example 56 using 2,4-difluoroaniline. ¹H NMR (400 MHz, CDCl₃), δ 8.23 (m, 1H), 7.43 (m, 5H), 7.02 (b, 1H), 6.79 (dd, 1H), 6.76 (m, 1H), 6.64 (m, 1H), 6.15 (dd, 1H), 5.82 (dd, 1H), 4.11 (t, 2H), 3.08 (t, 2H).

### EXAMPLE 71

### (E)-9-Phenyl-6,7-dihydro-5H-pyrrolo[1,2-a]azepine-8-carboxylic acid

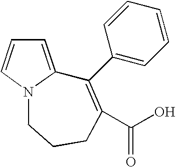

The compound (E)-9-phenyl-6,7-dihydro-5H-pyrrolo[1,2-a]azepine-8-carboxylic acid was prepared following the procedures described for Example 55 using benzoyl chloride and methyl 5-bromopentanoate. ¹H NMR (400 MHz, CDCl₃) δ 7.30 (m, 5H), 6.84 (t, 1H), 6.10 (dd, 1H), 5.82 (dd, 1H), 4.12 (t, 2H), 2.69 (t, 2H), 2.25 (m, 2H).

### EXAMPLE 72

### (E)-9-Phenyl-N-(4-(trifluoromethoxy)phenyl)-6,7-dihydro-5H-pyrrolo[1,2-a]azepine-8-carboxamide

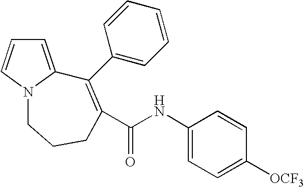

The compound (E)-9-phenyl-N-(4-(trifluoromethoxy)phenyl)-6,7-dihydro-5H-pyrrolo[1,2-a]azepine-8-carboxamide was prepared following the procedures described for Example 56 using 4-trifluoromethoxyaniline. ¹H NMR (400 MHz, CDCl₃) δ 7.37 (m, 5H), 7.02 (d, 2H), 6.96 (d, 2H), 6.87 (dd, 1H), 6.77 (s, 1H), 6.12 (dd, 1H), 5.85 (dd, 1H), 4.15 (t, 2H), 2.81 (t, 2H), 2.29 (m, 2H).

### EXAMPLE 73

### (E)-N-(2,4-Difluorophenyl)-9-phenyl-6,7-dihydro-5H-pyrrolo[1,2-a]azepine-8-carboxamide

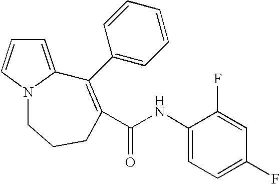

The compound (E)-N-(2,4-Difluorophenyl)-9-phenyl-6,7-dihydro-5H-pyrrolo[1,2-a]azepine-8-carboxamide was prepared following the procedures described for Example 56 using 2,4-difluoroaniline. ¹H NMR (400 MHz, CDCl₃) δ 8.13 (m, 1H), 7.32 (m, 5H), 6.93 (b, 1H), 6.85 (dd, 1H), 6.77 (m, 1H), 6.65 (m, 1H), 6.12 (dd, 1H), 5.84 (dd, 1H), 4.15 (t, 2H), 2.81 (t, 2H), 2.29 (m, 2H).

### EXAMPLE 74

### (E)-9-Phenyl-N-(4-(trifluoromethyl)phenyl)-6,7-dihydro-5H-pyrrolo[1,2-a]azepine-8-carboxamide

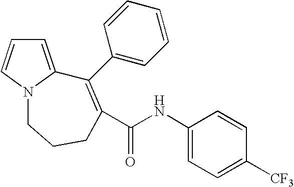

The compound (E)-9-phenyl-N-(4-(trifluoromethyl)phenyl)-6,7-dihydro-5H-pyrrolo[1,2-a]azepine-8-carboxamide was prepared following the procedures for Example 56 using 4-trifluoromethylaniline. ¹H NMR (400 MHz, CDCl₃) δ 7.41 (d, 2H), 7.38 (m, 5H), 7.07 (d, 2H), 6.88 (dd, 1H), 6.88 (s, 1H), 6.13 (dd, 1H), 5.86 (dd, 1H), 4.15 (t, 2H), 2.82 (t, 2H), 2.30 (m, 2H).

### EXAMPLE 75

### (E)-N-(3,4-Difluorophenyl)-9-phenyl-6,7-dihydro-5H-pyrrolo[1,2-a]azepine-8-carboxamide

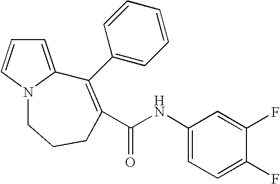

The compound (E)-N-(3,4-difluorophenyl)-9-phenyl-6,7-dihydro-5H-pyrrolo[1,2-a]azepine-8-carboxamide was prepared following the procedures for Example 56 using 3,4-difluoroaniline. ¹H NMR (400 MHz, CDCl₃) δ 7.30 (m, 5H), 7.13 (m, 1H), 6.99 (m, 1H), 6.87 (dd, 1H), 6.70 (s, 1H), 6.40 (m, 1H), 6.12 (dd, 1H), 5.85 (dd, 1H), 4.14 (t, 2H), 2.81 (t, 2H), 2.29 (m, 2H).

### EXAMPLE 76

### (E)-9-(4-tert-Butylphenyl)-6,7-dihydro-5H-imidazo[1,5-a]azepine-8-carboxylic acid

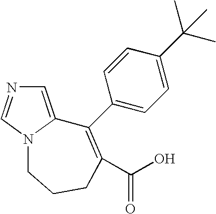

The compound (E)-9-(4-tert-butylphenyl)-6,7-dihydro-5H-imidazo[1,5-a]azepine-8-carboxylic acid was prepared following the procedures for Example 55 using imidazole and 4-tert-butylbenzoyl chloride. ¹H NMR (400 MHz, CD₃OD) δ 8.91 (s, 1H), 7.41(d, 2H), 7.24 (d, 2H), 6.95 (s, 1H), 4.48 (t, 2H), 2.74 (t, 2H), 2.34 (m, 2H), 1.32 (s, 9H).

### EXAMPLE 77

### (E)-9-(4-tert-Butylphenyl)-N-(4-chlorophenyl)-6,7-dihydro-5H-imidazo[1,5-a]azepine-8-carboxamide

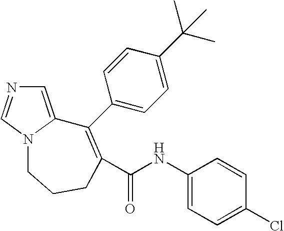

The compound (E)-9-(4-tert-butylphenyl)-N-(4-chlorophenyl)-6,7-dihydro-5H-imidazo[1,5-a]azepine-8-carboxamide was prepared following the procedures for Example 56 using (E)-9-(4-tert-butylphenyl)-6,7-dihydro-5H-imidazo[1,5-a]azepine-8-carboxylic acid and 4-chloroaniline. ¹H NMR (400 MHz, CDCl₃) δ 8.77 (s, 1H), 7.43 (d, 2H), 7.24 (d, 2H), 7.12 (d, 2H), 6.93 (s, 1H), 6.89 (s, 1H), 6.85 (d, 2H), 4.44 (t, 2H), 2.94 (t, 2H), 2.40 (m, 2H), 1.29 (s, 9H).

### EXAMPLE 78

### ((E)-9-(4-tert-Butylphenyl)-N-(2,4-difluorophenyl)-6,7-dihydro-5H-imidazo[1,5-a]azepine-8-carboxamide

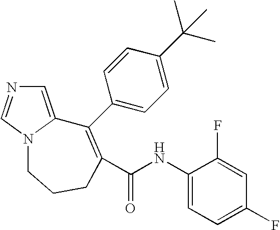

The compound ((E)-9-(4-tert-butylphenyl)-N-(2,4-difluorophenyl)-6,7-dihydro-5H-imidazo[1,5-a]azepine-8-carboxamide was prepared following the procedures for Example 56 using 2,4-difluoroaniline. ¹H NMR (400 MHz, CDCl₃) δ 8.88 (s, 1H), 7.91 (m, 1H), 7.39 (d, 2H), 7.24 (d, 2H), 7.17 (m, 2H), 6.93 (s, 1H), 6.77 (m, 1H), 4.47 (t, 2H), 2.94 (t, 2H), 2.40 (m, 2H), 1.27 (s, 9H).

### EXAMPLE 79

### (E)-9-(4-tert-Butylphenyl)-N-(3,4-dichlorophenyl)-6,7-dihydro-5H-imidazo[1,5-a]azepine-8-carboxamide

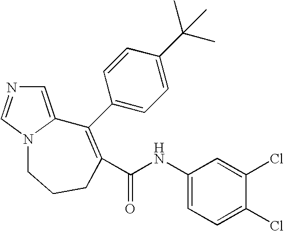

The compound (E)-9-(4-tert-butylphenyl)-N-(3,4-dichlorophenyl)-6,7-dihydro-5H-imidazo[1,5-a]azepine-8-carboxamide was prepared following the procedures for Example 56 using 3,4-dichloroaniline. ¹H NMR (400 MHz, CDCl₃) δ 8.88 (s, 1H), 7.65 (m, 1H), 7.50 (m, 1H), 7.44 (d, 2H), 7.24 (d, 2H), 7.10 (m, 1H), 7.04 (s, 1H), 6.94 (s, 1H), 4.48 (t, 2H), 2.94 (t, 2H), 2.41 (m, 2H), 1.30 (s, 9H).

### EXAMPLE 80

### (E)-9-(4-tert-Butylphenyl)-N-(2,4-dichlorophenyl)-6,7-dihydro-5H-imidazo[1,5-a]azepine-8-carboxamide

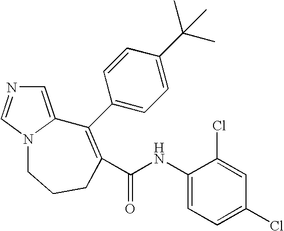

The compound (E)-9-(4-tert-butylphenyl)-N-(2,4-dichlorophenyl)-6,7-dihydro-5H-imidazo[1,5-a]azepine-8-carboxamide was prepared following the procedures for Example 56 using 2,4-dichloroaniline. ¹H NMR (400 MHz, CDCl₃) δ 9.03 (s, 1H), 8.20 (d, 1H), 7.38 (d, 2H), 7.42 (s, 1H), 7.22 (d, 2H), 7.19 (m, 2H), 7.01 (s, 1H), 4.50 (t, 2H), 2.94 (t, 2H), 2.41 (m, 2H), 1.26 (s, 9H).

### EXAMPLE 81

### (E)-9-(4-tert-Butylbenzyl)-6,7-dihydro-5H-imidazo[1,5-a]azepine-8-carboxylic acid

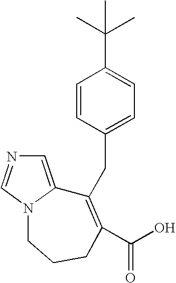

The compound (E)-9-(4-tert-butylbenzyl)-6,7-dihydro-5H-imidazo[1,5-a]azepine-8-carboxylic acid was prepared following the procedures for Example 55 using imidazole and 2-phenylacetyl chloride. ¹H NMR (400 MHz, CDCl₃) δ 8.97 (s, 1H), 7.24 (d, 2H), 7.09 (d, 2H), 6.92 (s, 1H), 4.16 (t, 2H), 3.98 (s, 2H), 2.51 (t, 2H), 2.31 (m, 2H), 1.27 (s, 9H).

### EXAMPLE 82

### (E)-9-(4-tert-Butylbenzyl)-N-(4-chlorophenyl)-6,7-dihydro-5H-imidazo[1,5-a]azepine-8-carboxamide

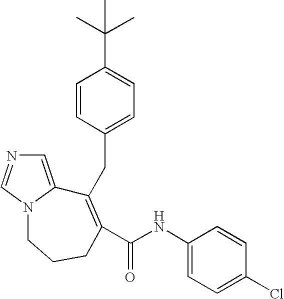

The compound (E)-9-(4-tert-butylbenzyl)-N-(4-chlorophenyl)-6,7-dihydro-5H-imidazo[1,5-a]azepine-8-carboxamide was prepared following the procedures for Example 56 using (E)-9-(4-tert-butylbenzyl)-6,7-dihydro-5H-imidazo[1,5-a]azepine-8-carboxylic acid and 4-chloroaniline. ¹H NMR (400 MHz, CDCl₃) δ 8.55 (s, 1H), 8.15 (s, 1H), 7.37 (d, 2H), 7.33 (d, 2H), 7.25 (d, 2H), 7.15 (d, 2H), 6.91 (s, 1H), 4.29 (t, 2H), 3.84 (s, 2H), 2.70 (t, 2H), 2.37 (m, 2H), 1.28 (s, 9H).

### EXAMPLE 83

### (E)-1-(9-(4-tert-Butylphenyl)-6,7-dihydro-5H-pyrrolo[1,2-a]azepin-8-yl)-3-(4-chlorophenyl)urea

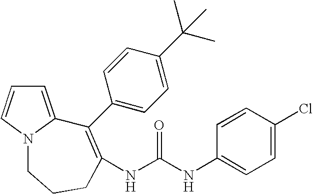

A mixture of (E)-9-(4-tert-butylphenyl)-6,7-dihydro-5H-pyrrolo[1,2-a]azepine-8-carboxylic acid (135 mg, 0.5 mmol), diphenylphosphoryl azide (130 μL, 0.6 mmol) and triethylamine (84 μL, 0.6 mmol) in benzene (2.5 mL) was stirred at room temperature overnight. p-Chloroaniline (76 mg, 0.6 mmol) was added and the mixture stirred at room temperature for 4 hours. The mixture was diluted with ethyl acetate and then washed with water, brine, dried over Na₂SO₄ and concentrated in vacuo. The crude material was purified by reverse phase HPLC to give 53 mg (25% yield) of the title compound. ¹H NMR (400 MHz, CDCl₃) δ 7.34 (d, 2H), 7.18 (d, 2H), 7.15 (d, 2H), 7.02 (d, 2H), 6.69 (dd, 1H), 6.24 (b, 1H), 6.22 (b, 1H), 6.04 (dd, 1H), 5.66 (dd, 1H), 4.12 (t, 2H), 2.79 (t, 2H), 2.35 (m, 2H), 1.32 (s, 9H).

### EXAMPLE 84

### (E)-1-(9-(4-tert-Butylphenyl)-6,7-dihydro-5H-pyrrolo[1,2-a]azepin-8-yl)-3-(2,4-difluorophenyl)urea

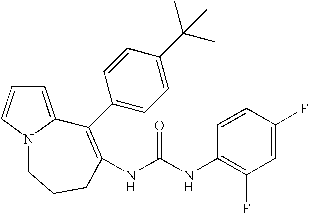

The compound (E)-1-(9-(4-tert-butylphenyl)-6,7-dihydro-5H-pyrrolo[1,2-a]azepin-8-yl)-3-(2,4-difluorophenyl)urea was prepared following the procedures for Example 83 using 2,4-difluoroaniline. ¹H NMR (400 MHz, CDCl₃) δ 7.67 (m, 1H), 7.34 (d, 2H), 7.33 (m, 1H), 7.15 (d, 2H), 6.78 (m, 1H), 6.70 (dd, 1H), 6.36 (b, 1H), 6.23 (b, 1H), 6.04 (dd, 1H), 5.67 (dd, 1H), 4.13 (t, 2H), 2.79 (t, 2H), 2.35 (m, 2H), 1.31 (s, 9H).

### EXAMPLE 85

### (E)-1-(9-(4-tert-Butylphenyl)-6,7-dihydro-5H-pyrrolo[1,2-a]azepin-8-yl)-3-(3,4-difluorophenyl)urea

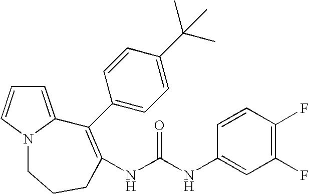

The compound (E)-1-(9-(4-tert-butylphenyl)-6,7-dihydro-5H-pyrrolo[1,2-a]azepin-8-yl)-3-(3,4-difluorophenyl)urea was prepared following the procedures for Example 83 using 3,4-difluoroaniline. ¹H NMR (400 MHz, CDCl₃) δ 7.36 (d, 2H), 7.32 (m, 1H), 7.15 (d, 2H), 6.96 (m, 2H), 6.70 (dd, 1H), 6.38 (b, 1H), 6.20 (b, 1H), 6.04 (dd, 1H), 5.67 (dd, 1H), 4.12 (t, 2H), 2.78 (t, 2H), 2.35 (m, 2H), 1.30 (s, 9H).

### EXAMPLE 86

### (E)-1-(9-(4-tert-Butylphenyl)-6,7-dihydro-5H-pyrrolo[1,2-a]azepin-8-yl)-3-(2,4-dichlorophenyl)urea

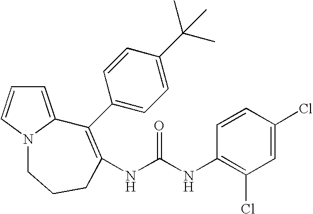

The compound (E)-1-(9-(4-tert-butylphenyl)-6,7-dihydro-5H-pyrrolo[1,2-a]azepin-8-yl)-3-(2,4-dichlorophenyl)urea was prepared following the procedures for Example 83 using 2,4-dichloroaniline. ¹H NMR (400 MHz, CDCl₃) δ 7.85 (d, 1H), 7.36 (d, 2H), 7.32 (m, 1H), 7.20 (d, 2H), 7.16 (m, 1H), 6.77 (s, 1H), 6.71 (dd, 1H), 6.16 (s, 1H), 6.04 (dd, 1H), 5.67 (dd, 1H), 4.15 (t, 2H), 2.77 (t, 2H), 2.33 (m, 2H), 1.30 (s, 9H).

### EXAMPLE 87

### (E)-1-(9-(4-tert-Butylphenyl)-6,7-dihydro-5H-pyrrolo[1,2-a]azepin-8-yl)-3-(3,4-dichlorophenyl)urea

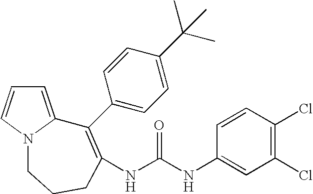

The compound (E)-1-(9-(4-tert-butylphenyl)-6,7-dihydro-5H-pyrrolo[1,2-a]azepin-8-yl)-3-(3,4-dichlorophenyl)urea was prepared following the procedures described for Example 83 using 3,4-dichloroaniline. ¹H NMR (400 MHz, CDCl₃) δ 7.35 (d, 2H), 7.34 (m, 1H), 7.23 (m, 1H), 7.18 (d, 2H), 6.93 (m, 1H), 6.70 (dd, 1H), 6.51 (b, 1H), 6.27 (b, 1H), 6.04 (dd, 1H), 5.68 (dd, 1H), 4.12 (t, 2H), 2.76 (t, 2H), 2.34 (m, 2H), 1.30 (s, 9H).

### EXAMPLE 88

### (E)-1-(9-(4-tert-Butylphenyl)-6,7-dihydro-5H-pyrrolo[1,2-a]azepin-8-yl)-3-(4-(trifluoromethyl)phenyl)urea

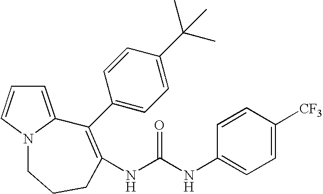

The compound (E)-1-(9-(4-tert-butylphenyl)-6,7-dihydro-5H-pyrrolo[1,2-a]azepin-8-yl)-3-(4-(trifluoromethyl)phenyl)urea was prepared following the procedures for Example 83 using 4-trifluoromethylaniline. ¹H NMR (400 MHz, CDCl₃) δ 7.46 (d, 2H), 7.36 (d, 2H), 7.27 (d, 2H), 7.19 (d, 2H), 6.71 (dd, 1H), 6.62 (b, 1H), 6.28 (b, 1H), 6.05 (dd, 1H), 5.69 (dd, 1H), 4.13 (t, 2H), 2.78 (t, 2H), 2.36 (m, 2H), 1.30 (s, 9H).

### EXAMPLE 89

### (E)-4-Methoxybenzyl 9-(4-tert-butylphenyl)-6,7-dihydro-5H-pyrrolo[1,2-a]azepin-8-ylcarbamate

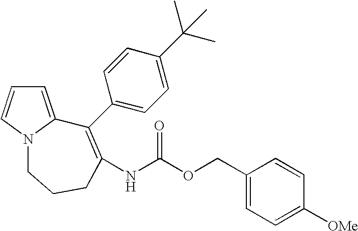

The compound (E)-4-methoxybenzyl 9-(4-tert-butylphenyl)-6,7-dihydro-5H-pyrrolo[1,2-a]azepin-8-ylcarbamate was prepared following the procedures for Example 83 using (4-methoxyphenyl)methanol. ¹H NMR (400 MHz, CDCl₃) δ 7.32 (d, 2H), 7.26 (d, 2H), 7.11 (d, 2H), 6.87 (d, 2H), 6.67 (dd, 1H), 6.45 (b, 1H), 6.02 (dd, 1H), 5.65 (dd, 1H), 5.00 (s, 2H), 4.08 (t, 2H), 3.80 (s, 3H), 2.83 (t, 2H), 2.31 (m, 2H), 1.31 (s, 9H).

### EXAMPLE 90

### 9-(4-tert-Butylphenyl)-6,7,8,9-tetrahydro-5H-pyrrolo[1,2-a]azepine-8-carboxylic acid

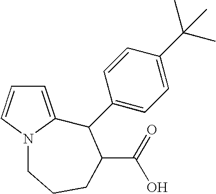

### Step 1

To a mixture of (E)-methyl 9-(4-tert-butylphenyl)-6,7-dihydro-5H-pyrrolo[1,2-a]azepine-8-carboxylate (1.07 g, 3.30 mmol) and NiCl₂.6H₂O (2.35 g, 9.91 mmol) in MeOH (110 mL) was added NaBH₄ (1.87 g, 49.5 mmol) in portions over 24 hours while stirring at room temperature. The mixture was concentrated in vacuo. The residue was taken up with 5% HCl and extracted with ethyl acetate twice. The combined organic layers were washed with saturated NaHCO₃, water, brine, dried over Na₂SO₄ and concentrated in vacuo. The crude material was purified by silica gel chromatography to give 1.05 g (98% yield) of methyl 9-(4-tert-butylphenyl)-6,7,8,9-tetrahydro-5H-pyrrolo[1,2-a]azepine-8-carboxylate as one diastereomer (presumably cis). ¹H NMR (400 MHz, CDCl₃) δ 7.25 (d, 2H), 6.80 (d, 2H), 6.56 (t, 1H), 6.04 (m, 2H), 5.05 (b, 1H), 3.90 (dd, 1H), 3.79 (s, 3H), 3.67 (t, 1H), 2.98 (td, 1H), 2.25 (m, 1H), 1.96 (m, 2H), 1.59 (m, 1H), 1.28 (s, 9H).

### Step 2

The ester from Step 1 was hydrolyzed with 1 N LiOH following the procedure for Example 55 to give a cis/trans (˜1.3:1) mixture of the title compound. ¹H NMR for presumed cis isomer (400 MHz, CDCl₃) δ 7.26 (d, 2H), 6.87 (d, 2H), 6.56 (t, 1H), 6.04 (m, 2H), 5.05 (b, 1H), 3.90 (dd, 1H), 3.66 (t, 1H), 3.02 (td, 1H), 2.24 (m, 1H), 1.96 (m, 2H), 1.59 (m, 1H), 1.28 (s, 9H). ¹H NMR for presumed trans isomer (400 MHz, CDCl₃) δ 7.30 (d, 2H), 7.25 (s, 1H), 7.13 (d, 2H), 6.56 (t, 1H), 5.92 (t, 1H), 5.47 (b, 1H), 4.37 (m, 1H), 3.99 (m, 2H), 3.16 (m, 1H), 2.07(m, 2H), 1.72 (m, 1H), 1.30 (s, 9H).

### EXAMPLE 91

### 9-(4-tert-Butylphenyl)-N-(3,4-difluorophenyl)-6,7,8,9-tetrahydro-5H-pyrrolo[1,2-a]azepine-8-carboxamide

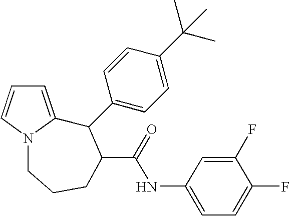

The compound 9-(4-tert-butylphenyl)-N-(3,4-difluorophenyl)-6,7,8,9-tetrahydro-5H-pyrrolo[1,2-a]azepine-8-carboxamide was prepared following the procedures for Example 56 using 3,4-difluoroaniline. Only one diastereomer (presumed trans) was isolated. ¹H NMR (400 MHz, CDCl₃) δ 7.44 (m, 2H), 7.35 (d, 2H), 7.23 (d, 2H), 6.98 (q, 1H), 6.77 (m, 2H), 6.10 (t, 1H), 5.63 (m, 1H), 4.43 (s, 1H), 4.20 (m, 1H), 4.09 (m, 1H), 3.26 (m, 1H), 2.62 (m, 1H), 2.04 (m, 1H), 1.76 (m, 1H), 1.31 (s, 9H).

### EXAMPLE 92

### 9-(4-tert-Butylphenyl)-N-(4-chlorophenyl)-6,7,8,9-tetrahydro-5H-pyrrolo[1,2-a]azepine-8-carboxamide

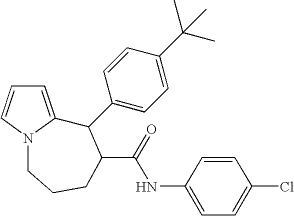

The compound 9-(4-tert-butylphenyl)-N-(4-chlorophenyl)-6,7,8,9-tetrahydro-5H-pyrrolo[1,2-a]azepine-8-carboxamide was prepared following the procedures for Example 56 using 4-chloroaniline. Only one diastereomer (presumed trans) was isolated. ¹H NMR (400 MHz, CDCl₃) δ 7.45 (s, 1H), 7.34 (d, 2H), 7.24 (m, 6H), 7.20 (m, 1H), 6.75 (t, 1H), 6.09 (t, 1H), 5.64 (m, 1H), 4.44 (s, 1H), 4.20 (m, 1H), 4.09 (m, 1H), 3.26 (dt, 1H), 2.61 (m, 1H), 2.01 (m, 1H), 1.77 (m, 1H), 1.30 (s, 9H).

### EXAMPLE 93

### 9-(4-tert-Butylphenyl)-N-(4-(trifluoromethyl)phenyl)-6,7,8,9-tetrahydro-5H-pyrrolo[1,2-a]azepine-8-carboxamide

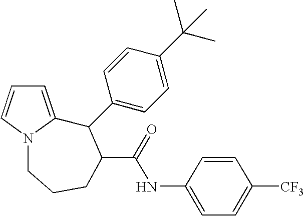

The compound 9-(4-tert-butylphenyl)-N-(4-(trifluoromethyl)phenyl)-6,7,8,9-tetrahydro-5H-pyrrolo[1,2-a]azepine-8-carboxamide was prepared following the procedures for Example 56 using 4-trifluoromethylaniline. Only one diastereomer (presumed trans) was isolated. ¹H NMR (400 MHz, CDCl₃) δ 7.60 (s, 1H), 7.49 (d, 2H), 7.40 (d, 2H), 7.37 (d, 2H), 7.23 (d, 2H), 6.77 (t, 1H), 6.11 (t, 1H), 5.66 (m, 1H), 4.45 (s, 1H), 4.20 (m, 1H), 4.09 (m, 1H), 3.28 (m, 1H), 2.62 (m, 1H), 2.04 (m, 2H), 1.74 (m, 1H), 1.30 (s, 9H).

### EXAMPLE 94

### 9-(4-tert-Butylphenyl)-N-(4-(trifluoromethoxy)phenyl)-6,7,8,9-tetrahydro-5H-pyrrolo[1,2-a]azepine-8-carboxamide

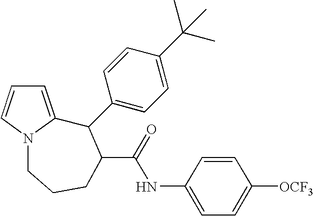

The compound 9-(4-tert-butylphenyl)-N-(4-(trifluoromethoxy)phenyl)-6,7,8,9-tetrahydro-5H-pyrrolo[1,2-a]azepine-8-carboxamide was prepared following the procedures described for Example 56 using 4-trifluoromethoxyaniline. Only one diastereomer (presumed trans) was isolated. ¹H NMR (400 MHz, CDCl₃) δ 7.50 (s, 1H), 7.35 (d, 2H), 7.31 (d, 2H), 7.24 (d, 2H), 7.09 (d, 2H), 6.76 (t, 1H), 6.09 (t, 1H), 5.64 (m, 1H), 4.44 (s, 1H), 4.20 (dd, 1H), 4.09 (m, 1H), 3.27 (m, 1H), 2.63 (m, 1H), 2.04 (m, 1H), 1.77 (m, 1H), 1.31 (s, 9H).

### EXAMPLE 95

### 8-(4-tert-Butylphenyl)-N-(3,4-difluorophenyl)-5,6,7,8-tetrahydroindolizine-7-carboxamide

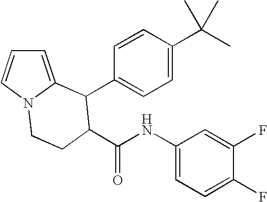

The compound 8-(4-tert-butylphenyl)-N-(3,4-difluorophenyl)-5,6,7,8-tetrahydroindolizine-7-carboxamide was prepared following the procedures for Example 56 using 3,4-difluoroaniline. A mixture of two diastereomers (cis/trans) was obtained after purification. MS (M/z, M+1): 409. Selected ¹H NMR data (400 MHz, CDCl₃) δ 6.67 (dd, 1H), 6.63 (m, 1H), 6.19 (t, 1H), 6.13 (t, 1H), 5.83 (m, 1H), 5.57 (m, 1H), 4.65 (d, 1H), 4.40 (m, 1H), 3.12 (m, 1H), 1.33 (s, 9H), 1.27 (s, 9H).

### EXAMPLE 96

### 8-(4-tert-Butylphenyl)-N-(4-(trifluoromethyl)phenyl)-5,6,7,8-tetrahydroindolizine-7-carboxamide

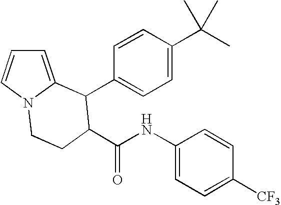

The compound 8-(4-tert-butylphenyl)-N-(4-(trifluoromethyl)phenyl)-5,6,7,8-tetrahydroindolizine-7-carboxamide was prepared following the procedures described for Example 56 using 4-trifluoromethylaniline. Only one diastereomer (presumed cis) was isolated. ¹H NMR (400 MHz, CDCl₃) δ 7.48 (d, 2H), 7.23 (m, 4H), 7.01 (d, 2H), 6.67 (dd, 1H), 6.57 (m, 1H), 6.20 (t, 1H), 5.84 (m, 1H), 4.66 (d 1H), 4.42 (m, 1H), 4.05 (m, 1H), 3.16 (m, 1H), 2.41 (m, 1H), 2.19 (m, 1H), 1.26 (s, 9H).

### EXAMPLE 97

### 8-(4-tert-Butylphenyl)-N-(4-(trifluoromethoxy)phenyl)-5,6,7,8-tetrahydroindolizine-7-carboxamide

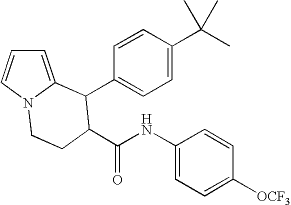

The compound 8-(4-tert-butylphenyl)-N-(4-(trifluoromethoxy)phenyl)-5,6,7,8-tetrahydroindolizine-7-carboxamide was prepared following the procedures for Example 56 using 4-trifluoromethoxyaniline. One diastereomer (presumed cis) was isolated. ¹H NMR (400 MHz, CDCl₃) δ 7.23 (d, 2H), 7.10 (m, 4H), 7.01 (d, 2H), 6.67 (dd, 1H), 6.50 (m, 1H), 6.20 (t, 1H), 5.83 (m, 1H), 4.66 (d 1H), 4.40 (m, 1H), 4.06 (m, 1H), 3.14 (m, 1H), 2.39 (m, 1H), 2.18 (m, 1H), 1.26 (s, 9H).

### EXAMPLE 98

### 8-(4-tert-Butylphenyl)-N-(4-chlorophenyl)-5,6,7,8-tetrahydroindolizine-7-carboxamide

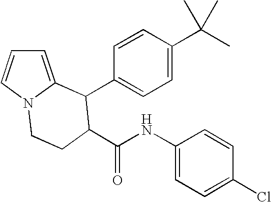

The compound 8-(4-tert-butylphenyl)-N-(4-chlorophenyl)-5,6,7,8-tetrahydroindolizine-7-carboxamide was prepared following the procedures described for Example 56 using 4-chloroaniline. A mixture of two diastereomers (cis/trans) was obtained. MS (M/z, M+1): 407. Selected ¹H NMR data (400 MHz, CDCl₃) δ 7.34 (d, 2H), 6.94 (d, 2H), 6.66 (dd, 1H), 6.63 (m, 1H), 6.47 (s, 1H), 6.29 (s, 1H), 6.19 (t, 1H), 6.13 (t, 1H), 5.83 (m, 1H), 5.57 (m, 1H), 4.65 (d, 1H), 4.40 (m, 1H), 4.13 (m, 4H), 3.12 (m, 1H), 2.64 (dt, 1H), 1.32 (s, 9H), 1.26 (s, 9H).

### EXAMPLE 99

### 8-Phenyl-5,6,7,8-tetrahydroindolizine-7-carboxylic acid

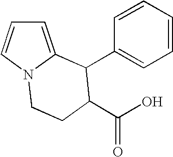

The compound 8-phenyl-5,6,7,8-tetrahydroindolizine-7-carboxylic acid was prepared following the procedures for Example 90. A mixture of two diastereomers (cis/trans ˜1:1.5) was obtained after purification. ¹H NMR for the presumed cis isomer (400 MHz, CDCl₃) δ 7.27 (m, 5H), 6.98 (d, 1H), 6.63 (dd, 1H), 6.17(t, 1H), 5.80 (m, 1H), 4.73 (d, 1H), 4.25 (m, 1H), 3.94 (m, 1H), 3.20 (m, 1H), 2.28 (m, 1H). ¹H NMR for the presumed trans isomer (400 MHz, CDCl₃) δ 7.28 (m, 5H), 7.00 (d, 1H), 6.59 (dd, 1H), 6.10 (t, 1H), 5.49 (m, 1H), 4.32 (d, 1H), 4.18 (m, 1H), 4.09 (m, 1H), 2.93 (dt, 1H), 2.28 (m, 1H).

### EXAMPLE 100

### N-(2,4-Difluorophenyl)-8-phenyl-5,6,7,8-tetrahydroindolizine-7-carboxamide

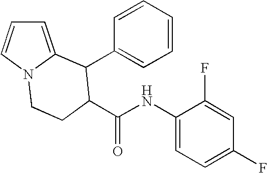

The compound N-(2,4-difluorophenyl)-8-phenyl-5,6,7,8-tetrahydroindolizine-7-carboxamide was prepared following the procedures described for Example 56 using 2,4-difluoroaniline. A mixture of two diastereomers (cis/trans) was obtained. MS (M/z, M+1): 353. Selected ¹H NMR data (400 MHz, CDCl₃) δ 8.02 (m, 2H), 6.20 (t, 1H), 6.13 (t, 1H), 5.83 (m, 1H), 5.53 (m, 1H), 4.70(d, 1H), 4.40 (m, 1H), 4.31 (d, 1H), 3.17 (m, 1H), 2.75 (dt, 1H).

### EXAMPLE 101

### N-(3,4-Difluorophenyl)-8-phenyl-5,6,7,8-tetrahydroindolizine-7-carboxamide

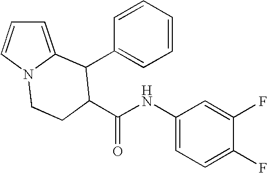

The compound N-(3,4-difluorophenyl)-8-phenyl-5,6,7,8-tetrahydroindolizine-7-carboxamide was prepared following the procedures for Example 56 using 3,4-difluoroanilline. A mixture of two diastereomers (cis/trans) was obtained. MS (M/z, M+1): 353. Selected ¹H NMR data (400 MHz, CDCl₃) δ 6.20 (t, 1H), 6.14 (t, 1H), 5.82 (m, 1H), 5.54 (m, 1H), 4.67 (d, 1H), 4.40 (m, 1H), 4.24 (d, 1H), 3.13 (m, 1H), 2.66 (dt, 1H).

### EXAMPLE 102

### N-(4-Chlorophenyl)-8-phenyl-5,6,7,8-tetrahydroindolizine-7-carboxamide

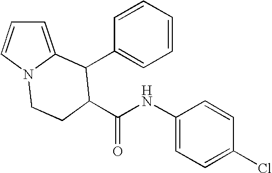

The compound N-(4-chlorophenyl)-8-phenyl-5,6,7,8-tetrahydroindolizine-7-carboxamide was prepared following the procedures for Example 56 using 4-chloroaniline. A mixture of two diastereomers (cis/trans) was obtained. MS (M/z, M+1): 351. Selected ¹H NMR data (400 MHz, CDCl₃) δ 6.67 (dd, 1H), 6.63 (m, 2H), 6.42 (s, 1H), 6.20 (t, 1H), 6.13 (t, 1H), 5.82 (m, 1H), 5.54 (m, 1H), 4.68 (d, 1H), 4.40 (m, 1H), 4.25 (d, 1H), 3.14 (m, 1H), 2.67 (dt, 1H).

### EXAMPLE 103

### 8-Phenyl-N-(4-(trifluoromethyl)phenyl)-5,6,7,8-tetrahydroindolizine-7-carboxamide

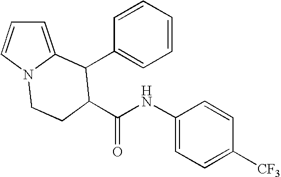

The compound 8-phenyl-N-(4-(trifluoromethyl)phenyl)-5,6,7,8-tetrahydroindolizine-7-carboxamide was prepared following the procedures described for Example 56 using 4-trifluoromethylaniline. A mixture of two diastereomers (cis/trans) was obtained. MS (M/z, M+1): 385. Selected ¹H NMR data (400 MHz, CDCl₃) δ 7.51 (d, 2H), 7.47 (d, 2H), 7.08 (d, 1H), 7.07 (d, 1H), 6.78 (m, 1H), 6.68 (dd, 1H), 6.64 (m, 1H), 6.55 (s, 1H), 6.20 (t, 1H), 6.13 (t, 1H), 5.82 (m, 1H), 5.54 (m, 1H), 4.68 (d, 1H), 4.40 (m, 1H), 4.26 (d, 1H), 3.14 (m, 1H), 2.67 (dt, 1H).

### EXAMPLE 104

### 8-Phenyl-N-(4-(trifluoromethoxy)phenyl)-5,6,7,8-tetrahydroindolizine-7-carboxamide

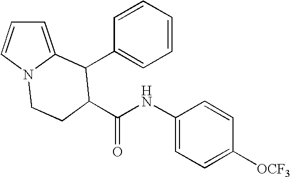

The compound 8-phenyl-N-(4-(trifluoromethoxy)phenyl)-5,6,7,8-tetrahydroindolizine-7-carboxamide was prepared following the procedures described for Example 56 using 4-trifluoromethoxyaniline. A mixture of two diastereomers (cis/trans) was obtained. MS (M/z, M+1): 401. Selected ¹H NMR data (400 MHz, CDCl₃) δ 6.68 (dd, 1H), 6.63 (m, 1H), 6.20 (t, 1H), 6.13 (t, 1H), 5.82 (m, 1H), 5.54 (m, 1H), 4.68 (d, 1H), 4.39 (m, 1H), 4.26 (m, 1H), 3.16 (m, 1H), 2.67 (dt, 1H).

### EXAMPLE 105

### (8,9-trans)-N-(2,4-Difluorophenyl)-9-phenyl-6,7,8,9-tetrahydro-5H-pyrrolo[1,2-a]azepine-8-carboxamide

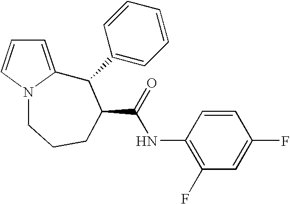

The compound (8,9-trans)-N-(2,4-difluorophenyl)-9-phenyl-6,7,8,9-tetrahydro-5H-pyrrolo[1,2-a]azepine-8-carboxamide was prepared following the procedures for Example 56 using 2,4-difluoroaniline. The cis and trans isomers were separated by prep TLC. The presumed trans isomer ¹H NMR (400 MHz, CDCl₃) δ 8.08 (m, 1H), 7.52 (b, 1H), 7.28 (m, 5H), 6.79 (m, 2H), 6.69 (t, 1H), 6.00 (t, 1H), 5.66 (m, 1H), 4.52 (s, 1H), 4.10 (m, 2H), 3.27 (m, 1H), 2.55 (m, 1H), 2.10 (m, 1H), 1.94 (m, 2H).

### EXAMPLE 106

### (8,9-cis)-N-(2,4-Difluorophenyl)-9-phenyl-6,7,8,9-tetrahydro-5H-pyrrolo[1,2-a]azepine-8-carboxamide

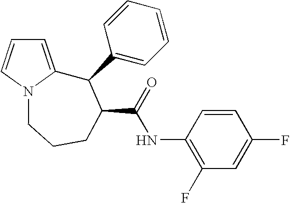

The compound (8,9-cis)-N-(2,4-difluorophenyl)-9-phenyl-6,7,8,9-tetrahydro-5H-pyrrolo[1,2-a]azepine-8-carboxamide was prepared following the procedures for Example 56 using 2,4-difluoroaniline. The cis and trans isomers were separated by prep TLC. The presumed cis isomer. ¹H NMR (400 MHz, CDCl₃) δ 7.72 (m, 1H), 7.28 (m, 5H), 7.12 (b, 1H), 6.74 (m, 2H), 6.62 (t, 1H), 5.96 (t, 1H), 5.54 (m, 1H), 4.41 (d, 1H), 4.08 (m, 2H), 3.00 (m, 1H), 2.22 (m, 2H), 2.07 (m, 1H), 1.78 (m, 1H).

### EXAMPLE 107

### (8,9-trans)-N-(3,4-Difluorophenyl)-9-phenyl-6,7,8,9-tetrahydro-5H-pyrrolo[1,2-a]azepine-8-carboxamide

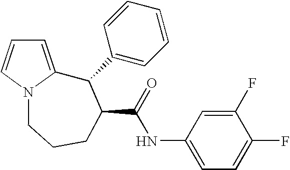

The compound (8,9-trans)-N-(3,4-difluorophenyl)-9-phenyl-6,7,8,9-tetrahydro-5H-pyrrolo[1,2-a]azepine-8-carboxamide was prepared following the procedures described for Example 56 using 3,4-difluoroaniline. The cis and trans isomers were separated by prep TLC. The presumed trans isomer ¹H NMR (400 MHz, CDCl₃) δ 7.46 (m, 1H), 7.30 (m, 5H), 6.98 (m, 2H), 6.77(m, 2H), 6.10 (t, 1H), 5.60 (m, 1H), 4.47 (s, 1H), 4.21 (dd,1H), 4.11 (m, 1H), 3.27 (m, 1H), 2.62 (m, 1H), 2.05 (m, 2H), 1.76 (m, 1H).

### EXAMPLE 108

### (8,9-cis)-N-(3,4-Difluorophenyl)-9-phenyl-6,7,8,9-tetrahydro-5H-pyrrolo[1,2-a]azepine-8-carboxamide

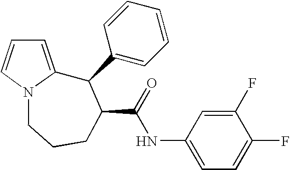

The compound (8,9-cis)-N-(3,4-difluorophenyl)-9-phenyl-6,7,8,9-tetrahydro-5H-pyrrolo[1,2-a]azepine-8-carboxamide was prepared following the procedures described for Example 56 using 3,4-difluoroaniline. The cis and trans isomers were separated by prep TLC. The presumed cis isomer. ¹H NMR (400 MHz, CDCl₃) δ 7.31 (m, 6H), 6.97 (q, 1H), 6.71 (m, 1H), 6.67 (m, 1H), 6.04 (s, 1H), 5.73 (b, 1H), 4.48 (d, 1H), 4.05 (m, 2H), 3.06 (m, 1H), 2.23 (m, 1H), 2.08 (m, 1H), 1.96 (m, 1H), 1.77 (m, 1H).

### EXAMPLE 109

### (8,9-trans)-N-(4-Chlorophenyl)-9-phenyl-6,7,8,9-tetrahydro-5H-pyrrolo[1,2-a]azepine-8-carboxamide

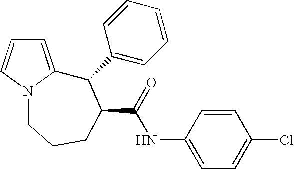

The compound (8,9-trans)-N-(4-chlorophenyl)-9-phenyl-6,7,8,9-tetrahydro-5H-pyrrolo[1,2-a]azepine-8-carboxamide was prepared following the procedures for Example 56 using 4-chloroaniline. The cis and trans isomers were separated by prep TLC. The presumed trans isomer ¹H NMR (400 MHz, CDCl₃) δ 7.42 (s, 1H), 7.30 (m, 5H), 7.10 (d, 2H), 6.76 (t, 1H), 6.61 (d, 2H), 6.09 (t, 1H), 5.60 (m, 1H), 4.47 (s, 1H), 4.20 (dd,1H), 4.10 (m, 1H), 3.26 (m, 1H), 2.62 (m, 1H), 2.05 (m, 2H), 1.78 (m, 1H).

### EXAMPLE 110

### (8,9-cis)-N-(4-Chlorophenyl)-9-phenyl-6,7,8,9-tetrahydro-5H-pyrrolo[1,2-a]azepine-8-carboxamide

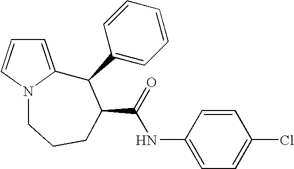

The compound (8,9-cis)-N-(4-chlorophenyl)-9-phenyl-6,7,8,9-tetrahydro-5H-pyrrolo[1,2-a]azepine-8-carboxamide was prepared following the procedures described for Example 56 using 4-chloroaniline. The cis and trans isomers were separated by prep TLC. The presumed cis isomer ¹H NMR (400 MHz, CDCl₃) δ 7.27 (m, 5H), 6.90 (d, 2H), 6.67 (t, 1H), 6.60 (d, 2H), 6.03 (m, 1H), 5.73 (b, 1H), 4.50 (d, 1H), 4.05 (m, 2H), 3.06 (m, 1H), 2.23 (m, 1H), 2.08 (m, 1H), 1.96 (m, 1H), 1.77 (m, 1H).

### EXAMPLE 111

### (8,9-trans)-9-Phenyl-N-(4-(trifluoromethyl)phenyl)-6,7,8,9-tetrahydro-5H-pyrrolo[1,2-a]azepine-8-carboxamide

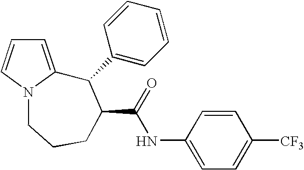

The compound (8,9-trans)-9-phenyl-N-(4-(trifluoromethyl)phenyl)-6,7,8,9-tetrahydro-5H-pyrrolo[1,2-a]azepine-8-carboxamide was prepared following the procedures described for Example 56 using 4-trifluoromethylaniline. The cis and trans isomers were separated by prep TLC. The presumed trans isomer ¹H NMR (400 MHz, CDCl₃) δ 7.56 (s, 1H), 7.49 (d, 2H), 7.40 (d, 2H), 7.30 (m, 5H), 6.77 (t, 1H), 6.11 (t, 1H), 5.62 (m, 1H), 4.48 (s, 1H), 4.21 (dd,1H), 4.12 (m, 1H), 3.29 (m, 1H), 2.62 (m, 1H), 2.05 (m, 2H), 1.78 (m, 1H).

### EXAMPLE 112

### (8,9-cis)-9-Phenyl-N-(4-(trifluoromethyl)phenyl)-6,7,8,9-tetrahydro-5H-pyrrolo[1,2-a]azepine-8-carboxamide

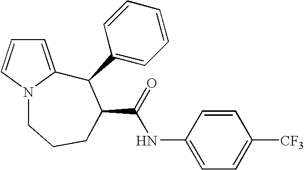

The compound (8,9-cis)-9-phenyl-N-(4-(trifluoromethyl)phenyl)-6,7,8,9-tetrahydro-5H-pyrrolo[1,2-a]azepine-8-carboxamide was prepared following the procedures described for Example 56 using 4-trifluoromethylaniline. A mixture of cis and trans were obtained. MS (M/z, M+1): 385. Selected ¹H NMR data (400 MHz, CDCl₃) δ 7.51 (d, 2H), 7.47 (d, 2H), 7.08 (d, 1H), 7.07 (d, 1H), 6.78 (m, 1H), 6.68 (dd, 1H), 6.64 (m, 1H), 6.55 (s, 1H), 6.20 (t, 1H), 6.13 (t, 1H), 5.82 (m, 1H), 5.54 (m, 1H), 4.68 (d, 1H), 4.40 (m, 1H), 4.26 (d, 1H), 3.14 (m, 1H), 2.67 (dt, 1H).

### EXAMPLE 113

### (8,9-trans)-9-Phenyl-N-(4-(trifluoromethoxy)phenyl)-6,7,8,9-tetrahydro-5H-pyrrolo[1,2-a]azepine-8-carboxamide

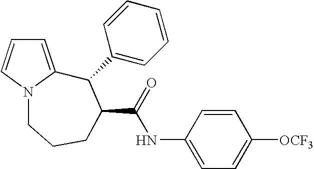

The compound (8,9-trans)-9-phenyl-N-(4-(trifluoromethoxy)phenyl)-6,7,8,9-tetrahydro-5H-pyrrolo[1,2-a]azepine-8-carboxamide was prepared following the procedures described for Example 56 using 4-trifluoromethoxyaniline. The cis and trans isomers were separated by prep TLC. The presumed trans isomer ¹H NMR (400 MHz, CDCl₃) δ 7.46 (s, 1H), 7.31 (m, 7H), 7.09 (d, 2H), 6.77 (t, 1H), 6.10 (t, 1H), 5.60 (m, 1H), 4.47 (s, 1H), 4.20 (dd,1H), 4.10 (m, 1H), 3.29 (m, 1H), 2.62 (m, 1H), 2.05 (m, 2H), 1.78 (m, 1H).

### EXAMPLE 114

### (8,9-cis)-9-Phenyl-N-(4-(trifluoromethoxy)phenyl)-6,7,8,9-tetrahydro-5H-pyrrolo[1,2-a]azepine-8-carboxamide

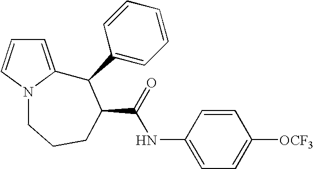

The compound (8,9-cis)-9-phenyl-N-(4-(trifluoromethoxy)phenyl)-6,7,8,9-tetrahydro-5H-pyrrolo[1,2-a]azepine-8-carboxamide was prepared following the procedures described for Example 56 using 4-trifluoromethoxyaniline. The cis and trans isomers were separated by prep TLC. ¹H NMR (400 MHz, CDCl₃) δ 7.29 (m, 7H), 7.20 (d, 2H), 7.06 (d, 2H), 6.67 (m, 1H), 6.04 (m, 1H), 5.73 (b, 1H), 4.50 (d, 1H), 4.05 (m, 2H), 3.08 (m, 1H), 2.23 (m, 1H), 2.10 (m, 1H), 1.96 (m, 1H), 1.78 (m, 1H).

### EXAMPLE 115

### 1-(9-(4-tert-Butylphenyl)-6,7,8,9-tetrahydro-5H-pyrrolo[1,2-a]azepin-8-yl)-3-(4-chlorophenyl)urea

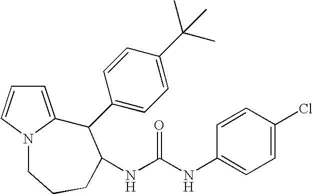

The compound 1-(9-(4-tert-butylphenyl)-6,7,8,9-tetrahydro-5H-pyrrolo[1,2-a]azepin-8-yl)-3-(4-chlorophenyl)urea was prepared following the procedures described for Example 83 using 4-chloroaniline. A mixture of two diastereomers (cis/trans) was obtained. MS (M/z, M+1): 436. Selected ¹H NMR data (400 MHz, CDCl₃) δ 7.37 (d, 1H), 7.33 (d, 2H), 7.27 (d, 2H), 7.19 (d, 2H), 6.98 (d, 2H), 6.85 (d, 2H), 6.62(m, 1H), 6.56 (t, 1H), 6.04 (m, 1H), 5.97 (dt, 1H), 1.31 (s, 9H), 1.28 (s, 9H).

### EXAMPLE 116

### 1-(9-(4-tert-Butylphenyl)-6,7,8,9-tetrahydro-5H-pyrrolo[1,2-a]azepin-8-yl)-3-(2,4-difluorophenyl)urea

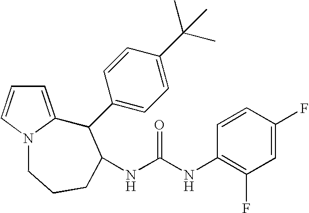

The compound 1-(9-(4-tert-butylphenyl)-6,7,8,9-tetrahydro-5H-pyrrolo[1,2-a]azepin-8-yl)-3-(2,4-difluorophenyl)urea was prepared following the procedures for Example 83 using 2,4-difluoroaniline. A mixture of two diastereomers (cis/trans) was obtained. MS (M/z, M+1): 438. Selected ¹H NMR data (400 MHz, CDCl₃) δ 7.74 (b, 1H), 7.38 (d, 1H), 7.31 (d, 2H), 6.97 (d, 2H), 6.62(m, 1H), 6.40 (b, 1H), 6.04 (m, 1H), 4.86 (s, 1H), 4.42 (d, 1H), 1.31 (s, 9H), 1.28 (s, 9H).

### EXAMPLE 117

### 1-(9-(4-tert-Butylphenyl)-6,7,8,9-tetrahydro-5H-pyrrolo[1,2-a]azepin-8-yl)-3-(3,4-difluorophenyl)urea

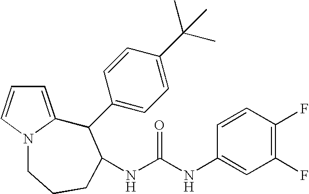

The compound 1-(9-(4-tert-butylphenyl)-6,7,8,9-tetrahydro-5H-pyrrolo[1,2-a]azepin-8-yl)-3-(3,4-difluorophenyl)urea was prepared following the procedure described for Example 83 using 3,4-difluoroaniline. A mixture of two diastereomers (cis/trans) was obtained. MS (M/z, M+1): 438. Selected ¹H NMR data (400 MHz, CDCl₃) δ 7.40 (d, 1H), 6.97 (m, 2H), 6.62(m, 1H), 6.60 (b, 1H), 6.04 (m, 1H), 1.31 (s, 9H), 1.28 (s, 9H).

### EXAMPLE 118

### 1-(9-(4-tert-Butylphenyl)-6,7,8,9-tetrahydro-5H-pyrrolo[1,2-a]azepin-8-yl)-3-(2,4-dichlorophenyl)urea

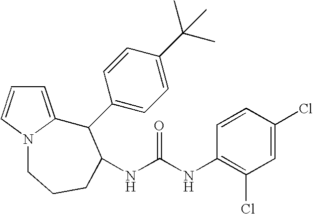

The compound 1-(9-(4-tert-butylphenyl)-6,7,8,9-tetrahydro-5H-pyrrolo[1,2-a]azepin-8-yl)-3-(2,4-dichlorophenyl)urea was prepared following the procedures described for Example 83 using 2,4-dichloroaniline. The two diastereomers (cis/trans) were separated by prep TLC. ¹H NMR (400 MHz, CDCl₃) δ 7.93 (b, 1H), 7.32 (m, 3H), 7.17 (dd, 1H), 6.98 (d, 2H), 6.66 (t, 1H), 6.60 (s, 1H), 6.07 (m, 2H), 4.91 (m, 2H), 4.44 (d, 1H), 3.94 (m, 1H), 3.77 (m, 1H), 2.01 (m, 1H), 1.88 (m, 1H), 1.72 (m, 2H), 1.30 (s, 9H).

### EXAMPLE 119

### 1-(9-(4-tert-Butylphenyl)-6,7,8,9-tetrahydro-5H-pyrrolo[1,2-a]azepin-8-yl)-3-(2,4-dichlorophenyl)urea

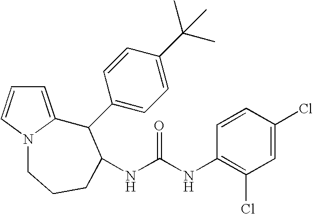

Diastereomer from Example 118. ¹H NMR (400 MHz, CDCl₃) δ 7.95 (d, 1H), 7.33 (m, 3H), 7.17 (m, 1H), 7.02 (d, 2H), 6.73 (m, 1H), 6.62 (s, 1H), 5.92 (t, 1H), 5.46 (m, 1H), 4.76 (d, 1H), 4.57 (m, 1H), 4.29 (s, 1H), 4.10 (m, 2H), 2.41 (m, 1H), 1.88 (m, 2H), 1.30 (s, 9H).

### EXAMPLE 120

### 1-(9-(4-tert-Butylphenyl)-6,7,8,9-tetrahydro-5H-pyrrolo[1,2-a]azepin-8-yl)-3-(3,4-dichlorophenyl)urea

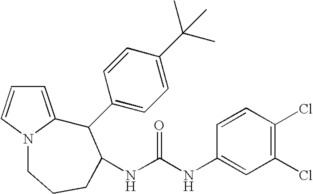

The compound 1-(9-(4-tert-butylphenyl)-6,7,8,9-tetrahydro-5H-pyrrolo[1,2-a]azepin-8-yl)-3-(3,4-dichlorophenyl)urea was prepared following the procedures described for Example 83 using 3,4-dichloroaniline. The two diastereomers (cis/trans) were separated by prep TLC. ¹H NMR (400 MHz, CDCl₃) δ 7.48 (b, 1H), 7.30 (m, 3H), 7.03 (m, 1H), 6.96 (d, 2H), 6.63 (t, 1H), 6.54 (s, 1H), 6.05 (m, 2H), 4.84 (m, 2H), 4.43 (d, 1H), 3.89 (m, 1H), 3.73 (m, 1H), 1.98 (m, 1H), 1.86 (m, 1H), 1.68 (m, 2H), 1.30 (s, 9H).

### EXAMPLE 121

### 1-(9-(4-tert-Butylphenyl)-6,7,8,9-tetrahydro-5H-pyrrolo[1,2-a]azepin-8-yl)-3-(3,4-dichlorophenyl)urea

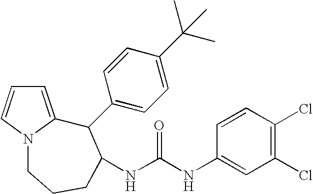

Diastereomer from Example 120. ¹H NMR (400 MHz, CDCl₃) δ 7.52 (b, 1H), 7.38 (m, 3H), 6.85 (m, 3H), 6.63 (m, 1H), 6.56 (s, 1H), 5.98 (t, 1H), 5.55 (m, 1H), 4.65 (m, 1H), 4.59 (m, 1H), 4.29 (s, 1H), 4.10 (m, 2H), 2.41 (m, 1H), 1.88 (m, 2H), 1.30 (s, 9H).

### EXAMPLE 122

### 1-(9-(4-tert-Butylphenyl)-6,7,8,9-tetrahydro-5H-pyrrolo[1,2-a]azepin-8-yl)-3-(4-(trifluoromethyl)phenyl)urea

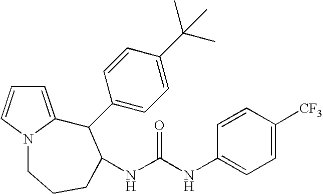

The compound 1-(9-(4-tert-Butylphenyl)-6,7,8,9-tetrahydro-5H-pyrrolo[1,2-a]azepin-8-yl)-3-(4-(trifluoromethyl)phenyl)urea was prepared following the procedures described for Example 83 using 4-trifluoromethylaniline. A mixture of two diastereomers (cis/trans) was obtained after purification. MS (M/z, M+1): 470. Selected ¹H NMR data (400 MHz, CDCl₃) δ 7.50 (d, 2H), 7.39 (m, 4H), 6.98 (m, 2H), 6.66 (m, 1H), 6.56 (b, 1H), 6.04 (m, 1H), 1.31 (s, 9H), 1.28 (s, 9H).

### EXAMPLE 123

### (9aS)-1-(4-tert-Butylbenzyl)octahydro-1H-pyrrolo[1,2-a][1,4]diazepine

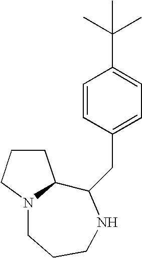

### Step 1

To a solution of (L)-N-BOC-Proline (10.8 g, 50.2 mmol) in CH₂Cl₂ (100 mL) was slowly added 1,1′-carbonyldiimidazole (9.76 g, 60.2 mmol) and stirred at room temperature for 15 minutes until CO₂ evolution ceases. N,O-Dimethylhydroxylamine hydrochloride (5.87 g, 60.2 mmol) was added and stirred at room temperature overnight. The mixture was diluted with water; the layers separated and extracted with CH₂Cl₂. The organic layers were washed with water, brine, dried over Na₂SO₄, and concentrated in vacuo to give 13.27 g (99% yield) of ((S)-tert-butyl 2-(methoxy(methyl)carbamoyl)pyrrolidine-1-carboxylate. ¹H NMR (400 MHz, CDCl₃) δ 4.65 (dd, 1H), 3.75 (d, 3H), 3.58 (m, 1H), 3.44 (m, 1H), 3.20 (s, 3H), 2.19(m, 1H), 2.00 (m, 1H), 1.86 (m, 2H), 1.44 (d, 9H).

### Step 2

To a suspension of magnesium powder (20 g, 821 mmol) in THF (50 mL) was added dibromoethane (80 μL, 82 mmol). The mixture was heated to reflux and then 4-tert-butylbenzyl bromide (15 mL, 82 mmol) was added in portions. After addition of the bromide, the resulting mixture was refluxed with stirring for 5 hours. The 4-tert-butylbenzyl magnesium bromide reagent was used directly in the next step.

### Step 3

A solution of ((S)-tert-butyl 2-(methoxy(methyl)carbamoyl)pyrrolidine-1-carboxylate (11.0 g, 42.6 mmol) in THF (50 mL) was cooled to 0° C. To the cold solution was added the 4-tert-butylbenzyl magnesium bromide solution (82 mmol) from Step 2. After addition, the cooling bath was removed and the mixture was stirred at room temperature for 6 hours. 3 N HCl was added to the mixture and extracted with ethyl acetate twice. The combined organic layers were washed with water, brine, dried over Na₂SO₄, and concentrated in vacuo. The crude material was purified by silica gel chromatography to give 7.5 g (50% yield) of (S)-tert-butyl-2-(2-(4-tert-butylphenyl)acetyl)pyrrolidine-1-carboxylate. ¹H NMR (400 MHz, CDCl₃) δ 7.35 (d, 2H), 7.14 (d, 2H), 4.40 (dd, 1H), 3.72 (s, 2H), 3.51 (m, 1H), 3.40 (t, 1H), 2.13 (m, 1H), 1.80 (m, 3H), 1.37 (s, 9H), 1.30 (s, 9H).

### Step 4

To neat (S)-tert-butyl-2-(2-(4-tert-butylphenyl)acetyl)pyrrolidine-1-carboxylate (5.33 g, 15.42 mmol) was added THF (5 mL), Ti(OiPr)₄ (5.9 mL, 19.28 mmol) and 3-aminopropan-1-ol (1.42 mL, 18.51 mmol). The mixture was stirred at room temperature under N₂ overnight. Dry MeOH (10 mL) was added to the mixture followed by excess NaBH₄ in portions. The mixture was stirred for 2 hours after addition of NaBH₄. 3 N NaOH (250 mL) was slowly added to the mixture and then filtered through Celite and washed with ethyl acetate. After separation of the filtrate, the aqueous layer was extracted with ethyl acetate and the combined organic layers were washed with brine, dried over Na₂SO₄, and concentrated in vacuo. The crude material was purified by silica gel chromatography to give 4.0 g (64% yield) of (2S)-tert-butyl-2-(2-(4-tert-butylphenyl)-1-(3-hydroxypropylamino)ethyl)pyrrolidine-1-carboxylate as a syn/anti mixture. MS (M/z, M+1): 405. Selected ¹H NMR data (400 MHz, CDCl₃) δ 7.32 (d, 2H), 7.16 (b, 2H), 4.13 (m, 1H), 3.65 (m, 2H), 2.80 (dd, 2H), 1.49(s, 9H), 1.30(s, 9H).

### Step 5

To (2S)-tert-butyl-2-(2-(4-tert-butylphenyl)-1-(3-hydroxypropylamino)ethyl)pyrrolidine-1-carboxylate (3.51 g, 8.68 mmol) in ethyl acetate (100 mL) was added concentrated HCl (˜43 mmol). The mixture was stirred at room temperature for 4 hours and then K₂CO₃ (8.6 g) was slowly added. After stirring for 30 minutes, the mixture was filtered and the filter cake was washed with ethyl acetate. The filtrate was washed with saturated NaHCO₃, dried over Na₂SO₄, and concentrated in vacuo to give 3.0 g (99% yield) of compound 3-(2-(4-tert-butylphenyl)-1-((S)-pyrrolidin-2-yl)ethylamino)propan-1-ol as a syn/anti mixture. MS (M/z, M+1): 305. Selected ¹H NMR data (400 MHz, CDCl₃) δ 7.34 (d, 2H), 7.13 (d, 2H), 3.76 (m, 2H), 3.56 (m, 1H), 3.39 (m, 1H), 2.95 (dd, 2H), 1.31 (s, 9H).

### Step 6

To a solution of 3-(2-(4-tert-butylphenyl)-1-((S)-pyrrolidin-2-yl)ethylamino)propan-1-ol (2.91 g, 9.56 mmol) in CH₂Cl₂ (50 mL) was added PPh₃ (2.5 g, 9.56 mmol) and NBS (1.7 g, 9.56 mmol). The mixture was stirred at room temperature for 4 hours. Triethylamine was added and the mixture stirred for 2 hours. The mixture was diluted with ethyl acetate and then washed with water, brine, dried over Na₂SO₄, and concentrated in vacuo. The crude material was purified by silica gel chromatography to give 4.0 g of the title compound as a cis/trans mixture. MS (M/z, M+1): 287. Selected ¹H NMR data (400 MHz, CDCl₃) δ 7.32 (d, 2H), 7.14 (d, 2H), 3.31 (m, 1H), 1.31 (s, 9H).

### EXAMPLE 124

### (9aS)-1-(4-tert-Butylbenzyl)-N-(2,4-difluorophenyl)hexahydro-1H-pyrrolo[1,2-a][1,4]diazepine-2(3H)-carboxamide

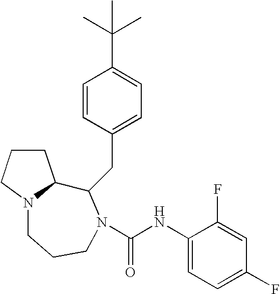

The compound (9aS)-1-(4-tert-butylbenzyl)-N-(2,4-difluorophenyl)hexahydro-1H-pyrrolo[1,2-a][1,4]diazepine-2(3H)-carboxamide was prepared following the procedures described for Example 2 using 2,4-difluoroisocyanate. A cis/trans mixture was obtained. MS (M/z, M+1): 442. Selected ¹H NMR data (400 MHz, CDCl₃) δ 7.67 (m, 1H), 7.32 (d, 2H), 7.17 (m, 1H), 7.10 (d, 2H), 6.69 (m, 1H), 6.42 (b, 1H), 4.63 (m, 1H), 4.35 (m, 1H), 3.98 (m, 1H), 3.79 (dd, 1H), 1.23 (s, 9H).

### EXAMPLE 125

### (9aS)-1-(4-tert-Butylbenzyl)-N-(4-chlorophenyl)hexahydro-1H-pyrrolo[1,2-a][1,4]diazepine-2(3H)-carboxamide

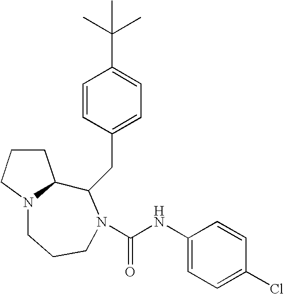

The compound (9aS)-1-(4-tert-butylbenzyl)-N-(4-chlorophenyl)hexahydro-1H-pyrrolo[1,2-a][1,4]diazepine-2(3H)-carboxamide was prepared following the procedures described for Example 2 using 4-chlorophenylisocyanate. A cis/trans mixture was obtained. MS (M/z, M+1): 440. Selected ¹H NMR data (400 MHz, CDCl₃) δ 7.34 (d, 2H), 7.30 (d, 2H), 7.18 (d, 2H), 7.15 (d, 2H), 7.09 (d, 2H), 7.05 (d, 2H), 6.94 (d, 2H), 6.81 (b, 1H), 6.78 (d, 2H), 5.95 (b, 1H), 5.00 (m, 1H), 4.62 (t, 1H), 4.45 (d, 1H), 4.09 (m, 1H), 3.91 (m, 2H), 1.29 (s, 9H), 1.23 (s, 9H).

### EXAMPLE 126

### (9aS)-1-(4-tert-Butylbenzyl)-N-phenylhexahydro-1H-pyrrolo[1,2-a][1,4]diazepine-2(3H)-carboxamide

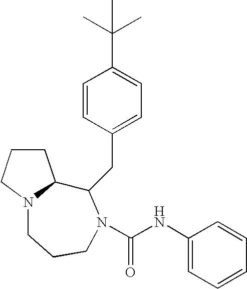

The compound (9aS)-1-(4-tert-butylbenzyl)-N-phenylhexahydro-1H-pyrrolo[1,2-a][1,4]diazepine-2(3H)-carboxamide was prepared following the procedures described for Example 2 using phenylisocyanate. A cis/trans mixture was obtained. MS (M/z, M+1): 406. Selected ¹H NMR data (400 MHz, CDCl₃) δ 7.33 (m, 2H), 7.21 (m, 2H), 7.18 (d, 2H), 7.15 (m, 3H), 6.95 (d, 2H), 6.90 (d, 2H), 6.00 (b, 1H), 5.05 (m, 1H), 4.57 (t, 1H), 4.30 (m, 1H), 4.09 (m, 1H), 3.24 (m, 2H), 1.30 (s, 9H), 1.26 (s, 9H).

### EXAMPLE 127

### (9aS)-1-(4-tert-Butylbenzyl)-N-(4-fluorophenyl)hexahydro-1H-pyrrolo[1,2-a][1,4]diazepine-2(3H)-carboxamide

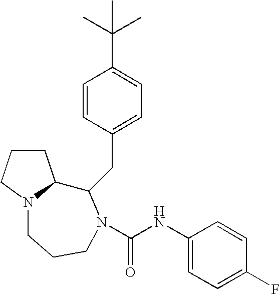

The compound (9aS)-1-(4-tert-butylbenzyl)-N-(4-fluorophenyl)hexahydro-1H-pyrrolo[1,2-a][1,4]diazepine-2(3H)-carboxamide was prepared following the procedures described for Example 2 using 4-fluorophenylisocyanate. A cis/trans mixture was obtained. MS (M/z, M+1): 424. Selected ¹H NMR data (400 MHz, CDCl₃) δ 7.34 (m, 4H), 7.18 (d, 2H), 7.13 (d, 2H), 6.90 (d, 4H), 6.79 (m, 2H), 6.72 (m, 2H), 6.51 (b, 1H), 5.89 (b, 1H), 5.05 (m, 1H), 4.61 (t, 1H), 4.42 (m, 1H), 4.11 (m, 1H), 1.29 (s, 9H), 1.25 (s, 9H).

### EXAMPLE 128

### (9aS)-1-(4-tert-Butylphenyl)octahydro-1H-pyrrolo[1,2-a][1,4]diazepine

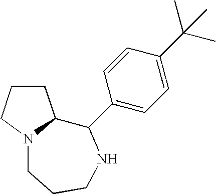

The compound (9aS)-1-(4-tert-butylphenyl)octahydro-1H-pyrrolo[1,2-a][1,4]diazepine was prepared following the procedures described for Example 123 using 4-tert-butylphenyl magnesium bromide ¹H NMR (400 MHz, CDCl₃) δ 7.34 (q, 4H), 4.02 (d, 1H), 3.42 (m, 1H), 3.23 (m, 3H), 2.82 (m, 3H), 1.97 (m, 1H), 1.84 (m, 1H), 1.60 (m, 4H), 1.32 (s, 9H).

### EXAMPLE 129

### (9aS)-1-(4-tert-Butylphenyl)-N-(2,4-difluorophenyl)hexahydro-1H-pyrrolo[1,2-a][1,4]diazepine-2(3H)-carboxamide

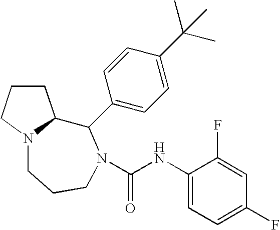

The compound (9aS)-1-(4-tert-butylphenyl)-N-(2,4-difluorophenyl)hexahydro-1H-pyrrolo[1,2-a][1,4]diazepine-2(3H)-carboxamide was prepared following the procedures described for Example 2 using 2,4-difluorophenylisocyanate. ¹H NMR (400 MHz, CDCl₃) δ 7.98 (m, 1H), 7.57 (d, 2H), 7.36 (d, 2H), 6.77 (m, 2H), 6.52 (b, 1H), 5.16 (s, 1H), 4.22 (m, 1H), 3.29 (m, 1H), 3.15 (m, 1H), 3.08 (m, 1H), 3.02 (t, 1H), 2.37 (m, 2H), 1.99 (m, 2H), 1.68 (m, 1H), 1.55 (m, 3H), 1.32 (s, 9H).

### EXAMPLE 130

### (9aS)-1-(4-tert-Butylphenyl)-N-(3,4-difluorophenyl)hexahydro-1H-pyrrolo[1,2-a][1,4]diazepine-2(3H)-carboxamide

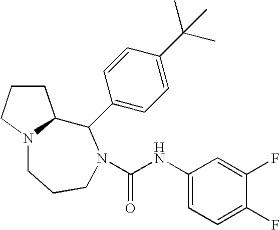

The compound (9aS)-1-(4-tert-butylphenyl)-N-(3,4-difluorophenyl)hexahydro-1H-pyrrolo[1,2-a][1,4]diazepine-2(3H)-carboxamide was prepared following the procedures described for Example 2 using 3,4-difluorophenylisocyanate. ¹H NMR (400 MHz, CDCl₃) δ 7.54 (d, 2H), 7.40 (d, 2H), 7.10 (m, 1H), 6.96 (m, 1H), 6.66 (m, 1H), 6.54 (m, 1H), 4.92 (s, 1H), 4.41 (m, 1H), 3.27 (m, 1H), 3.18 (m, 2H), 2.97 (m, 1H), 2.36 (m, 2H), 1.98 (m, 4H), 1.70 (m, 1H), 1.48 (m, 3H), 1.32 (s, 9H).

### EXAMPLE 131

### (9aS)-1-(4-tert-Butylphenyl)-N-(4-chlorophenyl)hexahydro-1H-pyrrolo[1,2-a][1,4]diazepine-2(3H)-carboxamide

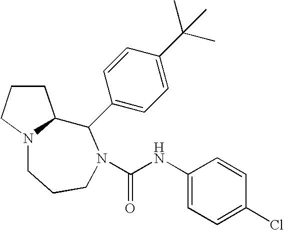

The compound (9aS)-1-(4-tert-butylphenyl)-N-(4-chlorophenyl)hexahydro-1H-pyrrolo[1,2-a][1,4]diazepine-2(3H)-carboxamide was prepared following the procedures described for Example 2 using 4-chlorophenylisocyanate. ¹H NMR (400 MHz, CDCl₃) δ 7.55 (d, 2H), 7.39 (d, 2H), 7.13 (d, 2H), 7.00 (d, 2H), 6.41 (s, 1H), 4.99 (s, 1H), 4.38 (m, 1H), 3.26 (ddd, 1H), 3.16 (m, 2H), 2.98 (t, 1H), 2.36 (m, 2H), 1.98 (m, 4H), 1.67 (m, 1H), 1.48 (m, 3H), 1.33 (s, 9H).

### EXAMPLE 132

### 8-(4-tert-Butylphenyl)-N-(4-(trifluoromethyl)phenyl)-5,6-dihydroindolizine-7-carboxamide

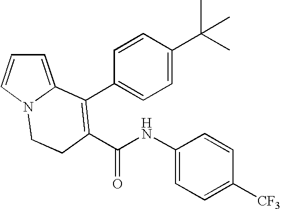

The compound 8-(4-tert-butylphenyl)-N-(4-(trifluoromethyl)phenyl)-5,6-dihydroindolizine-7-carboxamide was prepared following the procedures described for Example 56 using 4-trifluoromethylaniline. ¹H NMR (400 MHz, CDCl₃) δ 7.77 (dd, 1H), 7.54 (d, 2H), 7.43 (m, 2H), 7.36 (d, 2H), 6.97 (d, 2H), 6.81 (dd, 1H), 6.17 (dd, 1H), 5.92 (dd, 1H), 4.14 (t, 2H), 3.09 (t, 2H), 1.39 (s, 9H).

### EXAMPLE 133

### 5-(4-tert-Butylphenyl)-6,7,8,9-tetrahydro-5H-pyrido[3,2-c]azepine

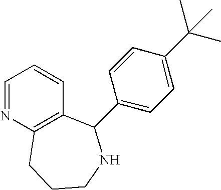

### Step 1

Isobenzofuran-1,3-dione (4.44 g, 30.0 mmol) was dissolved in toluene (50 mL) and 4-aminobutanoic acid (3.09, 30.0 mmol) added. Triethylamine (0.5 mL) was added dropwise and the mixture heated to 140° C. for 4 hours. The mixture was cooled to 0° C. and filtered. The filter cake was washed with hexane to give 4-(1,3-dioxoisoindolin-2-yl)butanoic acid (3 g, 43%).

### Step 2

Dimethyl carbonate (9.0 g, 100.0 mmol) was added to a solution of NaH (1.2 g, 50.0 mmol) in THF (250 mL) under N₂. 1-(4-tert-Butylphenyl)ethanone (8.8 g, 50.0 mmol) in THF (50 mL) was added dropwise at 65° C. The mixture was heated at that temperature for 3 hours. Then H₂O/ice (300 mL) was added. The mixture was extracted with EtOAc. The organic layers were then washed with brine, dried, and concentrated in vacuo. The crude material was purified by silica gel chromatography to give methyl 3-(4-tert-butylphenyl)-3-oxopropanoate (10 g, 85%).

### Step 3

4-(1,3-Dioxoisoindolin-2-yl)butanoic acid (466 mg, 2.0 mmol) was dissolved in SOCl₂ (10 mL) and the mixture heated to reflux for 3 hours. The mixture was cooled to room temperature and concentrated in vacuo. Methyl 3-(4-tert-butylphenyl)-3-oxopropanoate (468 mg, 2.0 mmol) was dissolved in THF (20 mL) and MgCl₂ (188 mg, 2.0 mmol) was added. The mixture was cooled to −30° C. and pyridine (316 mg, 4.0 mmol) was added dropwise. The mixture was stirred cold for 1 hour then the acid chloride prepared earlier was added in THF (10 mL). The mixture was stirred cold for 3 hours. H₂O/ice (10 mL) was added followed by 2 N HCl until the pH=5. The mixture was extracted with EtOAc. The combined organic layers were washed with brine, dried, and concentrated in vacuo. The crude material was purified by silica gel chromatography to give methyl 2-(4-tert-butylbenzoyl)-6-(1,3dioxoisoindolin-2-yl)-3-oxohexanoate (200 mg, 22%).

### Step 4

Methyl 2-(4-tert-butylbenzoyl)-6-(1,3dioxoisoindolin-2-yl)-3-oxohexanoate (20 g, 44.5 mmol) was dissolved in DMSO (200 mL). LiCl (1.87 g, 44.5 mmol) was added followed by H₂O (800 mg, 44.4 mmol). The mixture was stirred at 160° C. for 45 minutes. The mixture was cooled to room temperature and H₂O (400 mL) was added. The mixture was extracted with EtOAc three times. The combined organic layers were washed with brine, dried over Na₂SO₄, and concentrated in vacuo. The crude material was purified by silica gel chromatography to give 2-(6-(4-tert-butylphenyl)-4,6-dioxohexyl)isoindoline-1,3-dione.

### Step 5

2-(6-(4-tert-Butylphenyl)-4,6-dioxohexyl)isoindoline-1,3-dione (7 g, 17.9 mmol) was dissolved in toluene (100 mL) and ammonium acetate (13.8 g, 179.0 mmol) was added followed by acetic acid (2 mL). The mixture was heated to 140° C. for 5 hours under Dean Stark conditions. The mixture was cooled to room temperature and diluted with H₂O. The mixture was extracted with EtOAc. The combined organic layers were washed with NaHCO₃, brine, dried, and concentrated in vacuo to give (Z)-2-(4-amino-6-(4-tert-butylphenyl)-6-oxohex-4-enyl)isoindoline-1,3-dione (6 g). The crude material was used without further purification.

### Step 6

(Z)-2-(4-Amino-6-(4-tert-butylphenyl)-6-oxohex-4-enyl)isoindoline-1,3-dione (6 g, 15.4 mmol) was dissolved in toluene (72 mL) and acetic acid (17 mL). 1,1,3,3-Tetraethoxypropane (3.38 g, 15.4 mmol) was added followed by four drops of H₂O. The mixture was heated at 130° C. overnight. The mixture was cooled to room temperature and the solvent removed in vacuo. The residue was taken up in EtOAc and extracted with NaHCO₃, dried over Na₂SO₄, and concentrated. The crude material was purified by silica gel chromatography to give 2-(3-(3-(4-tert-butylbenzoyl)-pyridin-2-yl)propyl)isoindoline-1,3-dione (1.1 g).

### Step 7

6 M HCl (40 mL) was added to 2-(3-(3-(4-tert-butylbenzoyl)-pyridin-2-yl)propyl)isoindoline-1,3-dione (1.0 g, 2.35 mmol). The mixture was heated at 130° C. overnight. After cooling to room temperature; 6 M NaOH was added until the pH=9. The mixture was diluted with H₂O (30 mL) and extracted with EtOAc three times. The combined organic layers were washed with brine, dried over Na₂SO₄, and concentrated in vacuo. The crude material was purified by silica gel chromatography to give (Z)-5-(4-tert-butylphenyl)-8,9-dihydro-7H-pyrido[3,2-c]azepine (300 mg).

### Step 8

(Z)-5-(4-tert-Butylphenyl)-8,9-dihydro-7H-pyrido[3,2-c]azepine (300 mg, 1.08 mmol) was dissolved in MeOH (20 mL) and NaBH₄ (45 mg, 1.18 mmol) was added in portions at 0° C. After 1 hour, 2 N HCl (1 mL) was added. Na₂CO₃ (sat. aq.) was added until the pH=8. The mixture was diluted with H₂O and extracted with EtOAc three times. The combined organic layers were washed with brine, dried over Na₂SO₄, and concentrated in vacuo. The crude material was purified by silica gel chromatography followed by recrystallization from hexane to give the title compound (80 mg, 27%). ¹H NMR (300 MHz, CDCl₃) δ 8.31 (d, 1H), 7.41 (d, 2H), 7.24 (d, 2H), 6.93 (m, 2H), 5.10 (s, 1H), 3.30 (m, 4H), 2.16 (b, 1H), 1.90 (m, 1H), 1.75 (m, 1H), 1.34 (s, 9H).

### EXAMPLE 134

### 5-(4-tert-Butylphenyl)-N-(2,4-difluorophenyl)-8,9-dihydro-5H-pyrido[3,2-c]azepine-6(7H)-carboxamide

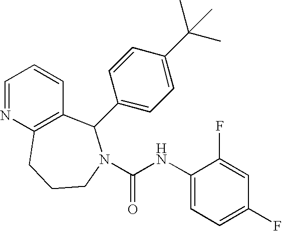

The compound 5-(4-tert-butylphenyl)-N-(2,4-difluorophenyl)-8,9-dihydro-5H-pyrido[3,2-c]azepine-6(7H)-carboxamide was prepared following the procedures described for Example 2 using 5-(4-tert-butylphenyl)-6,7,8,9-tetrahydro-5H-pyrido[3,2-c]azepine and 2,4-difluorophenylisocyanate. ¹H NMR (400 MHz, CDCl₃) δ 8.46 (d, 1H), 8.01 (m, 1H), 7.42 (d, 2H), 7.31 (m, 1H), 7.13 (m, 1H), 7.10 (d, 2H), 6.81 (m, 2H), 6.62 (s, 1H), 6.50 (b, 1H), 3.75 (m, 2H), 3.13 (m, 2H), 1.85 (m, 2H), 1.34 (s, 9H).

### EXAMPLE 135

### 5-(4-tert-Butylphenyl)-N-(3,4-difluorophenyl)-8,9-dihydro-5H-pyrido[3,2-c]azepine-6(7H)-carboxamide

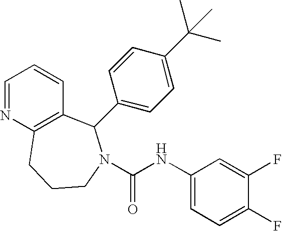

The compound 5-(4-tert-butylphenyl)-N-(3,4-difluorophenyl)-8,9-dihydro-5H-pyrido[3,2-c]azepine-6(7H)-carboxamide was prepared following the procedures described for Example 2 using 5-(4-tert-butylphenyl)-6,7,8,9-tetrahydro-5H-pyrido[3,2-c]azepine and 3,4-difluoropehnylisocyanate. ¹H NMR (400 MHz, CDCl₃) δ 8.45 (d, 1H), 7.42 (d, 2H), 7.34 (m, 2H), 7.13 (m, 1H), 7.08 (d, 2H), 7.00 (m, 1H), 6.82 (m, 1H), 6.61 (m, 1H), 6.52 (s, 1H), 3.74 (m, 2H), 3.12 (m, 2H), 1.85 (m, 2H), 1.34 (s, 9H).

### EXAMPLE 136

### 5-(4-tert-Butylphenyl)-N-(2,4-dichlorophenyl)-8,9-dihydro-5H-pyrido[3,2-c]azepine-6(7H)-carboxamide

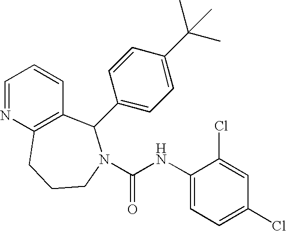

The compound 5-(4-tert-butylphenyl)-N-(2,4-dichlorophenyl)-8,9-dihydro-5H-pyrido[3,2-c]azepine-6(7H)-carboxamide was prepared following the procedures described for Example 2 using 5-(4-tert-butylphenyl)-6,7,8,9-tetrahydro-5H-pyrido[3,2-c]azepine and 2,4-dichlorophenylisocyanate. ¹H NMR (400 MHz, CDCl₃) δ 8.46 (d, 1H), 8.14 (d, 1H), 7.43 (d, 2H), 7.27 (m, 2H), 7.19 (dd, 1H), 7.12 (d, 2H), 7.09 (m, 2H), 6.47 (b, 1H), 3.75 (m, 2H), 3.13 (m, 2H), 1.85 (m, 2H), 1.34 (s, 9H).

### EXAMPLE 137

### 5-(4-tert-Butylphenyl)-6,7,8,9-tetrahydro-5H-pyrido[3,2-c]azepine

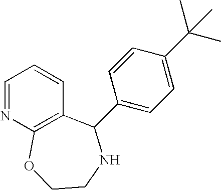

### Step 1

2-Chloronicotinic acid (10 g, 64.1 mmol) was dissolved in SOCl₂ (200 mL). The mixture was refluxed for 4 hours. The mixture was cooled to room temperature and concentrated in vacuo to give 2-chloronicotinoyl chloride. The crude material was used in the next step without further purification.

### Step 2

Dry ether (40 mL) was added to Mg (1.53 g, 63.8 mmol) under N₂. 1-Bromo-4-tert-butylbenzene (13.5 g, 63.7 mmol) was added dropwise at reflux. The mixture was stirred at reflux for 30 minutes. The mixture was cooled to −20° C. and 2-chloronicotinoyl chloride (10 g, 57.5 mmol) in THF (50 mL) was added. The mixture was stirred cold for 30 minutes, then brought to reflux for an additional hour. H₂O/ice (50 mL) was added followed by K₂CO₃ (sat. aq.) until the pH=7. The mixture was extracted with EtOAc and concentrated. The crude material was purified by silica gel chromatography to give (4-tert-butylphenyl)(2-chloropyridin-3-yl)methanone (7.5 g, 43%).

### Step 3

(4-tert-Butylphenyl)(2-chloropyridin-3-yl)methanone (5.5 g, 20.1 mmol) was dissolved in ethanol (100 mL). 2-Aminoethanol (5.0 g, 82.0 mmol) was added. The mixture was refluxed for 24 hours. The mixture was concentrated and purified by silica gel chromatography to give (E)-2-((4-tert-butylphenyl)(2-chloropyridin-3-yl)methyleneamino)ethanol (2.5 g, 39%).

### Step 4

(E)-2-((4-tert-Butylphenyl)(2-chloropyridin-3-yl)methyleneamino)ethanol (2.5 g, 7.91 mmol) was dissolved in ethanol (100 mL) and NaBH₄ (10 g, 263 mmol) was added in portions at 0° C. The mixture was warmed to room temperature for 4 hours. The mixture was concentrated and the residue taken up in H₂O. The aqueous mixture was extracted with EtOAc, dried, and concentrated in vacuo. The crude material was purified by silica gel chromatography to give 2-((4-tert-butylphenyl)(2-chloropyridin-3-yl)methylamino)ethanol (0.6 g, 24%).

### Step 5

2-((4-tert-Butylphenyl)(2-chloropyridin-3-yl)methylamino)ethanol (600 mg, 1.89 mmol) was dissolved in THF (40 mL) and NaH (370 mg, 9.25 mmol) was added. The mixture was heated to reflux for 4 hours. The mixture was concentrated and the residue taken up in H₂O. The aqueous mixture was extracted with EtOAc three times. The combined organic layers were dried and concentrated in vacuo. The crude material was purified by silica gel chromatography to give the title compound (300 mg, 56%). ¹H NMR (300 MHz, D₂O) δ 8.22 (d, 1H), 7.52 (d, 2H), 7.46 (s, 1H), 7.23 (d, 2H), 5.94 (s, 1H), 4.47 (m, 1H), 3.65 (m, 3H), 1.75 (m, 1H), 1.20 (s, 9H).

### EXAMPLE 138

### 5-(4-tert-Butylphenyl)-N-(2,4-difluorophenyl)-2,3-dihydropyrido[3,2-f][1,4]oxazepine-4(5H)-carboxamide

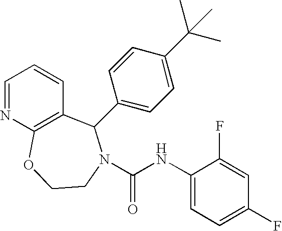

The compound 5-(4-tert-butylphenyl)-N-(2,4-difluorophenyl)-2,3-dihydropyrido[3,2-f][1,4]oxazepine-4(5H)-carboxamide was prepared following the procedures for Example 2 using 5-(4-tert-butylphenyl)-6,7,8,9-tetrahydro-5H-pyrido[3,2-c]azepine and 2,4-difluorophenylisocyanate. ¹H NMR (400 MHz, CDCl₃) δ 8.34 (m, 1H), 7.98 (m, 1H), 7.69 (d, 1H), 7.39 (d, 2H), 7.16 (m, 1H), 7.07 (d, 2H), 6.87 (m, 2H), 6.77 (s, 1H), 6.57 (d, 1H), 4.40 (m, 1H), 4.21 (m, 1H), 4.04 (m, 1H), 3.60 (m, 1H), 1.31 (s, 9H).

### EXAMPLE 139

### 5-(4-tert-Butylphenyl)-N-(3,4-difluorophenyl)-2,3-dihydropyrido[3,2-f][1,4]oxazepine-4(5H)-carboxamide

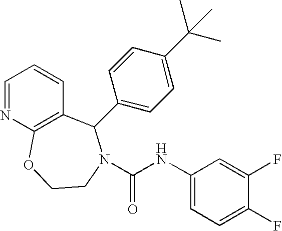

The compound 5-(4-tert-butylphenyl)-N-(3,4-difluorophenyl)-2,3-dihydropyrido[3,2-f][1,4]oxazepine-4(5H)-carboxamide was prepared following the procedures for Example 2 using 5-(4-tert-butylphenyl)-6,7,8,9-tetrahydro-5H-pyrido[3,2-c]azepine and 3,4-difluorophenylisocyanate. ¹H NMR (400 MHz, CDCl₃) δ 8.31 (dd, 1H), 7.72 (dd, 1H), 7.42 (m, 1H), 7.38 (d, 2H), 7.17 (dd, 1H), 7.05 (m, 1H), 7.03 (d, 2H), 6.90 (s, 1H), 6.83 (d, 1H), 4.36 (m, 1H), 4.16 (td, 1H), 4.06 (m, 1H), 3.83 (b, 1H), 3.54 (m, 1H), 1.31 (s, 9H).

### EXAMPLE 140

### 5-(4-tert-Butylphenyl)-N-(2,4-dichlorophenyl)-2,3-dihydropyrido[3,2-f][1,4]oxazepine-4(5H)-carboxamide

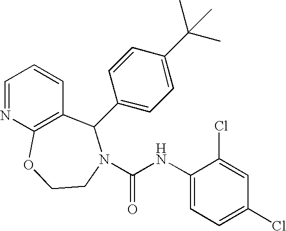

The compound 5-(4-tert-butylphenyl)-N-(2,4-dichlorophenyl)-2,3-dihydropyrido[3,2-f][1,4]oxazepine-4(5H)-carboxamide was prepared following the procedures for Example 2 using 5-(4-tert-butylphenyl)-6,7,8,9-tetrahydro-5H-pyrido[3,2-c]azepine and 2,4-dichlorophenylisocyanate. ¹H NMR (400 MHz, CDCl₃) δ 8.35 (dd, 1H), 8.14 (d, 1H), 7.70 (dd, 1H), 7.40 (d, 2H), 7.35 (d, 1H), 7.23 (dd, 1H), 7.17 (dd, 1H), 7.08 (d, 2H), 6.73 (s, 1H), 4.41 (m, 1H), 4.25 (dq, 1H), 4.08 (m, 1H), 3.73 (b, 1H), 3.62 (m, 1H), 1.31 (s, 9H).

The following compounds can generally be made using the methods described above. It is expected that these compounds when made will have activity similar to those that have been made in the examples above.

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as many IUPAC names, were generated using CambridgeSoft's ChemDraw 10.0.
Cc1ccccc1C2N(Cc3cc(cc(c3)C(F)(F)F)C(F)(F)F)CCCn4cccc24
Cc1ccccc1C2N(CCCn3cccc23)S(═O)(═O)c4cc(cc(c4)C(F)(F)F)C(F)(F)F
Cc1ccccc1C2N(CCCn3ccnc23)C(═O)c4cc(cc(c4)C(F)(F)F)C(F)(F)F
Cc1ccccc1C2N(CCCn3ccnc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4ccnc24
Cc1ccccc1C2N(CCCn3cncc23)C(═O)c4cc(cc(c4)C(F)(F)F)C(F)(F)F
Cc1ccccc1C2N(CCCn3cncc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4cncc24
Cc1ccccc1C2N(CCCn3nccc23)C(═O)c4cc(cc(c4)C(F)(F)F)C(F)(F)F
Cc1ccccc1C2N(CCCn3nccc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4nccc24
Cc1ccccc1C2N(CCCn3ncnc23)C(═O)c4cc(cc(c4)C(F)(F)F)C(F)(F)F
Cc1ccccc1C2N(CCCn3ncnc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4ncnc24
Cc1ccccc1C2N(CCCn3cnnc23)C(═O)c4cc(cc(c4)C(F)(F)F)C(F)(F)F
Cc1ccccc1C2N(CCCn3cnnc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4cnnc24
Cc1ccccc1C2N(CCCn3nnnc23)C(═O)c4cc(cc(c4)C(F)(F)F)C(F)(F)F
Cc1ccccc1C2N(CCCn3nnnc23)S(═O)(═O)c4cc(cc(c4)C(F)(F)F)C(F)(F)F
Cc1ccccc1C2N(Cc3cc(cc(c3)C(F)(F)F)C(F)(F)F)CCCn4nnnc24
FC(F)(F)c1cc(CN2CCCn3cccc3C2c4ccccc4)cc(c1)C(F)(F)F
FC(F)(F)c1cc(cc(c1)S(═O)(═O)N2CCCn3cccc3C2c4ccccc4)C(F)(F)F
FC(F)(F)c1cc(cc(c1)C(F)(F)F)C(═O)N2CCCn3ccnc3C2c4ccccc4
FC(F)(F)c1cc(cc(c1)S(═O)(═O)N2CCCn3ccnc3C2c4ccccc4)C(F)(F)F
FC(F)(F)c1cc(CN2CCCn3ccnc3C2c4ccccc4)cc(c1)C(F)(F)F
FC(F)(F)c1cc(cc(c1)C(F)(F)F)C(═O)N2CCCn3cncc3C2c4ccccc4
FC(F)(F)c1cc(cc(c1)S(═O)(═O)N2CCCn3cncc3C2c4ccccc4)C(F)(F)F
FC(F)(F)c1cc(CN2CCCn3cncc3C2c4ccccc4)cc(c1)C(F)(F)F
FC(F)(F)c1cc(cc(c1C(F)(F)F)C(═O)N2CCCn3nccc3C2c4ccccc4
FC(F)(F)c1cc(cc(c1)S(═O)(═O)N2CCCn3nccc3C2c4ccccc4)C(F)(F)F
FC(F)(F)c1cc(CN2CCCn3nccc3C2c4ccccc4)cc(c1)C(F)(F)F
FC(F)(F)c1cc(cc(c1)C(F)(F)F)C(═O)N2CCCn3ncnc3C2c4ccccc4
FC(F)(F)c1cc(cc(c1)S(═O)(═O)N2CCCn3ncnc3C2c4ccccc4)C(F)(F)F
FC(F)(F)c1cc(CN2CCCn3ncnc3C2c4ccccc4)cc(c1)C(F)(F)F
FC(F)(F)c1cc(cc(c1)C(F)(F)F)C(═O)N2CCCn3cnnc3C2c4ccccc4
FC(F)(F)c1cc(cc(c1)S(═O)(═O)N2CCCn3cnnc3C2c4ccccc4)C(F)(F)F
FC(F)(F)c1cc(CN2CCCn3cnnc3C2c4ccccc4)cc(c1)C(F)(F)F
FC(F)(F)c1cc(cc(c1)C(F)(F)F)C(═O)N2CCCn3nnnc3C2c4ccccc4
FC(F)(F)c1cc(cc(c1)S(═O)(═O)N2CCCn3nnnc3C2c4ccccc4)C(F)(F)F
FC(F)(F)c1cc(CN2CCCn3nnnc3C2c4ccccc4)cc(c1)C(F)(F)F
O═C(NC1=CC═CC═C1)N2CCCn3cccc3C2C4=CC═C(C(C)(C)C)C═C4
CC(C)(C)C(C═C1)=CC═C1C2N(S(NC3=CC═CC═C3)(═O)═O)CCCn4cccc24
O═C(CC1=CC═CC═C1)N2CCCn3cccc3C2C4=CC═C(C(C)(C)C)C═C4
O═C(NC1=CC═CC═C1)N2CCCn3nccc3C2C4=CC═C(C(C)(C)C)C═C4
CC(C)(C)C(C═C1)=CC═C1C2N(S(NC3=CC═CC═C3)(=O)═O)CCCn4nccc24
O═C(CC1=CC═CC═C1)N2CCCn3nccc3C2C4=CC═C(C(C)(C)C)C═C4
O═C(C1=CC═CC═C1)N2CCCn3nccc3C2C4=CC═C(C(C)(C)C)C═C4
O═C(NC1=CC═CC═C1)N2CCCn3cncc3C2C4=CC═C(C(C)(C)C)C═C4
CC(C)(C)C(C═C1)=CC═C1C2N(S(NC3=CC═CC═C3)(=O)═O)CCCn4cncc24
O═C(CC1=CC═CC═C1)N2CCCn3cncc3C2C4=CC═C(C(C)(C)C)C═C4
O═C(C1=CC═CC═C1)N2CCCn3cncc3C2C4=CC═C(C(C)(C)C)C═C4
O═C(NC1=CC═CC═C1)N2CCCn3ccnc3C2C4=CC═C(C(C)(C)C)C═C4
CC(C)(C)C(C═C1)=CC═C1C2N(S(NC3=CC═CC═C3)(=O)═O)CCCn4ccnc24
O═C(CC1=CC═CC═C1)N2CCCn3ccnc3C2C4=CC═C(C(C)(C)C)C═C4
O═C(C1=CC═CC═C1)N2CCCn3ccnc3C2C4=CC═C(C(C)(C)C)C═C4
O═C(NC1=CC═CC═C1)N2CCCn3ncnc3C2C4=CC═C(C(C)(C)C)C═C4
CC(C)(C)C(C═C1)=CC═C1C2N(S(NC3=CC═CC═C3)(=O)═O)CCCn4ncnc24
O═C(CC1=CC═CC═C1)N2CCCn3ncnc3C2C4=CC═C(C(C)(C)C)C═C4
O═C(C1=CC═CC═C1)N2CCCn3ncnc3C2C4=CC═C(C(C)(C)C)C═C4
O═C(NC1=CC═CC═C1)N2CCCn3ncnc3C2C4=CC═C(C(C)(C)C)C═C4
CC(C)(C)C(C═C1)=CC═C1C2N(S(NC3=CC═CC═C3)(=O)═O)CCCn4ncnc24
O═C(CC1=CC═CC═C1)N2CCCn3ncnc3C2C4=CC═C(C(C)(C)C)C═C4
O═C(C1=CC═CC═C1)N2CCCn3ncnc3C2C4=CC═C(C(C)(C)C)C═C4
O═C(NC1=CC═CC═C1)N2CCCn3ccsc3C2C4=CC═C(C(C)(C)C)C═C4
CC(C)(C)C(C═C1)=CC═C1C2N(S(NC3=CC═CC═C3)(=O)═O)CCCn4ccsc24
O═C(CC1=CC═CC═C1)N2CCCn3ccsc3C2C4=CC═C(C(C)(C)C)C═C4
O═C(C1=CC═CC═C1)N2CCCn3ccsc3C2C4=CC═C(C(C)(C)C)C═C4
O═C(NC1=CC═CC═C1)N2CCCn3cscc3C2C4=CC═C(C(C)(C)C)C═C4
CC(C)(C)C(C═C1)=CC═C1C2N(S(NC3=CC═CC═C3)(=O)═O)CCCn4cscc24
O═C(CC1=CC═CC═C1)N2CCCn3cscc3C2C4=CC═C(C(C)(C)C)C═C4
O═C(C1=CC═CC═C1)N2CCCn3cscc3C2C4=CC═C(C(C)(C)C)C═C4
O═C(NC1=CC═CC═C1)N2CCCc3ccsc3C2C4=CC═C(C(C)(C)C)C═C4
CC(C)(C)C(C═C1)=CC═C1C2N(S(NC3=CC═CC═C3)(=O)═O)CCCc4ccsc24
O═C(CC1=CC═CC═C1)N2CCCc3ccsc3C2C4=CC═C(C(C)(C)C)C═C4
O═C(C1=CC═CC═C1)N2CCCc3ccsc3C2C4=CC═C(C(C)(C)C)C═C4
O═C(NC1=CC═CC═C1)N2CCCc3sccc3C2C4=CC═C(C(C)(C)C)C═C4
CC(C)(C)C(C═C1)=CC═C1C2N(S(NC3=CC═CC═C3)(=O)═O)CCCc4sccc24
O═C(CC1=CC═CC═C1)N2CCCc3sccc3C2C4=CC═C(C(C)(C)C)C═C4
O═C(C1=CC═CC═C1)N2CCCc3sccc3C2C4=CC═C(C(C)(C)C)C═C4
O═C(NC1=CC═CC═C1)N2CCCn3cocc3C2C4=CC═C(C(C)(C)C)C═C4
O═C(NC1=CC═CC═C1)N2CCCc3cocn3C2C4=CC═C(C(C)(C)C)C═C4
O═C(NC1=CC═CC═C1)N2CCCc3ccoc3C2C4=CC═C(C(C)C)C═C4
O═C(NC1=CC═CC═C1)N2CCCc3occc3C2C4=CC═C(C(C)(C)C)C═C4
O═C(NC1=CC═CC═C1)N2CCCn3nnnc3C2C4=CC═C(C(C)(C)C)C═C4
O═C(NC1=CC═CC2=C1C═CC═C2)N3CCCn4cccc4C3C5=CC═C(C(C)(C)C)C═C5
O═C(NC1=CC═CS1)N2CCCn3cccc3C2C4=CC═C(C(C)(C)C)C═C4
O═C(NC1=NC═CN1)N2CCCn3cccc3C2C4=CC═C(C(C)(C)C)C═C4
O═C(NC1=CC═CC═C1)N2CCCn3cccc3C2C4=CC═C(C(C)C)C═C4
O═C(NC1=CC═CC═C1)N2CCCn3cccc3C2C4=CC═C(C(C)CC)C═C4
O═C(NC1=CC═CC═C1)N2CCCn3cccc3C2C4=CC═C(C)C═C4
O═C(NC1=CC═CC═C1)N2CCCn3cccc3C2C4=CC═C(C5=CC═CC═C5)C═C4
O═C(NC1=CC═CC═C1)N2CCCn3cccc3C2C4=CC═C(C(F)(F)F)C═C4
O═C(NC1=CC═CC═C1)N2CCCn3cccc3C2C4=CC═C(F)C═C4
O═C(NC1=CC═CC═C1)N2CCCn3cccc3C2C4=CC═C(OC(F)(F)F)C═C4
O═C(NC1=CC═CC═C1)N2CCCn3cccc3C2C4=CC═C(C═CC═C5)C5=C4
O═C(NC1=CC═CC═C1)N2CCCn3cccc3C2C4=CC═C(C═CS5)C5=C4
O═C(NC1=CC═CC═C1)N2CCCn3cccc3C2C4=CC═C(C═CN5)C5=C4
O═C(NC1=CC═CC═C1)N2CCCn3cccc3C2C4=CC(C═CC═C5)=C5S4
O═C(NC1=CC═CC═C1)N2CCCn3cccc3C2C4=CC(C═CC═C5)=C5N4
O═C(NC1=CC═CC═C1)N2CCCN3C(CCC3)C2C4=CC═C(C(C)(C)C)C═C4
O═C(NC1=CC═CC═C1)N2CCCn3cccc3C2C4CCC(C(C)(C)C)CC4
O═C(NC1=CC═CC═C1)N2CCCN3C(CCC3)C2C4CCC(C(C)(C)C)CC4
O═C(NC1=CC═CC═C1)N2CCCn3cccc3C2C4=CC═C(C(C)(C)C)C═N4
O═C(NC1=CC═CC═C1)N2CCCn3cccc3C2C4=CN═C(C(C)(C)C)C═C4
O═C(NC1=CC═CC═N1)N2CCCn3cccc3C2C4=CC═C(C(C)(C)C)C═C4
O═C(NC1=CN═CC═C1)N2CCCn3cccc3C2C4=CC═C(C(C)(C)C)C═C4
O═C(NC1=CC═NC═C1)N2CCCn3cccc3C2C4=CC═C(C(C)(C)C)C═C4
O═C(NC1=CC═CC═C1)C2CCCn3cccc3C2C4=CC═C(C(C)(C)C)C═C4
O═S(C1CCCn2cccc2C1C3=CC═C(C(C)(C)C)C═C3)(NC4=CC═CC═C4)=O
CC(C)(C)C(C═C1)=CC═C1C2C(CNC3=CC═CC═C3)CCCn4cccc24
CC(C)(C)C(C═C1)=CC═C1C2C(NC(C3=CC═CC═C3)=O)CCCn4cccc24
O═C(NC1=CC═CC═C1)N2CCn3cccc3C2C4=CC═C(C(C)(C)C)C═C4
O═C(NC1=CC═CC═C1)N2CCCCn3cccc3C2C4=CC═C(C(C)(C)C)C═C4
O═C(N1CCCCn2c(C1C3=CC═C(C═C3)C(C)(C#N)C)ccc2)NC4=CC═CC═C4
O═C(N1CCCCn2c(C1C3=CC═C(C═C3)CC#N)ccc2)NC4=CC═CC═C4
O═C(N1CCCCn2c(C1C3=CC═C(C═C3)C(CC)(CC)C#N)ccc2)NC4=CC═CC═C4
O═C(N1CCCCn2c(C1C3=CC═C(C═C3)C(C)(C)C(OC)═O)ccc2)NC4=CC═CC═C4
O═C(N1CCCCn2c(C1C3=CC═C(C═C3)C(C)(C)C(NC)═O)ccc2)NC4=CC═CC═C4
O═C(N1CCCCn2c(C1C3=CC═C(C═C3)C(C)(C)C(N(C)C)═O)ccc2)NC4=CC═CC═C4
O═C(N1CCCCn2c(C1C3=CC═C(C═C3)C(C)(C)C(N)═O)ccc2)NC4=CC═CC═C4
O═C(N1CCCCn2c(C1C3=CC═C(C═C3)C(C)(C)C(N)═O)ccc2)NC4=CC═CC═C4
O═C(N1CCCCn2c(C1C3=CC═C(C═C3)C(C)(C)CN(C)C)ccc2)NC4=CC═CC═C4
O═C(N1CCCCn2c(C1C3=CN═C(C═C3)C(C)(C#N)C)ccc2)NC4=CC═CC═C4
O═C(N1CCCCn2c(C1C3=NC═C(C═C3)C(C)(C#N)C)ccc2)NC4=CC═CC═C4
O═C(N1CCCCC2=NC═CC═C2C1C3=CC═C(C═C3)C(C)(C#N)C)NC4=CC═CC═C4
O═C(N1CCCCC2=CN═CC═C2C1C3=CC═C(C═C3)C(C)(C#N)C)NC4=CC═CC═C4
O═C(N1CCCCC2=CC═NC═C2C1C3=CC═C(C═C3)C(C)(C#N)C)NC4=CC═CC═C4
O═C(N1CCCCC2=NC═NC═C2C1C3=CC═C(C═C3)C(C)(C#N)C)NC4=CC═CC═C4
O═C(N1CCCCC2=NC═CN═C2C1C3=CC═C(C═C3)C(C)(C#N)C)NC4=CC═CC═C4
O═C(N1CCCCC2=CC═CN═C2C1C3=CC═C(C═C3)C(C)(C#N)C)NC4=CC═CC═C4
O═C(N1CCCCC2=NN═CC═C2C1C3=CC═C(C═C3)C(C)(C#N)C)NC4=CC═CC═C4
O═C(N1CCCN2C(C1C3=CC═C(C═C3)C(C)(C#N)C)═CC═C2)NC4=CC═CC═C4
O═C(N1CCCN2C(C1C3=CC═C(C═C3)C(C)(C#N)C)═CC═N2)NC4=CC═CC═C4
O═C(N1CCCN2C(C1C3=CC═C(C═C3)C(C)(C#N)C)═CN═C2)NC4=CC═CC═C4
O═C(N1CCCN2C(C1C3=CC═C(C═C3)C(C)(C#N)C)═NC═C2)NC4=CC═CC═C4

The activity of the compounds in Examples 1-140 as CB2 modulators is illustrated in the following assays. The other compounds listed above, which have not yet been made and/or tested, are predicted to have activity in these assays as well.

### Biological Activity Assays

### 1. Human CB2 Radioligand Binding Assay

This receptor binding filtration assay measures the receptor-ligand interactions of compounds by measuring ability to compete with a radiolabeled control ligand. Reactions were performed in 96 deep-well plate (Costar 3961) with a final volume of 600 ul. Compounds were added to each well either in single point (10 uM final) or dose-response. Compounds were delivered in DMSO or further diluted in incubation buffer to a final assay DMSO concentration of 1%. Incubation buffer (50 mM Tris-HCl pH 7.4, 2.5 mM EGTA, 5 mM MgCl₂, 5.0 mg/ml fatty acid free BSA) was added to each well. Following buffer addition, [³H] CP-55,940 was added. Tritiated CP-55,940 (Perkin Elmer) was used at the Kd determined from prior saturation binding experiments conducted on each lot of membranes. Membrane preparations from stably transfected CHO CB2 cells were added and the reaction was incubated for 90 minutes at 30° C. Unifilter GF/C filtration plates (Perkin Elmer) were pre-wet with 0.05% polyethylenimine. Samples were transferred to Unifilter plates and separation of unbound radioligand was achieved using a vacuum manifold (Millipore). Following thorough washing with 5 ml/well ice-cold wash buffer (50 mM Tris-HCl pH 7.4, 2.5 mM EGTA, 5 mM MgCl₂, 0.5 mg/ml fatty acid free BSA), filterplates were dried completely. Betaplate Scint Scintillation cocktail (25 ul, Perkin Elmer) was applied and plates were read using Microbeta Trilux (Wallac). Data analysis was performed in Spotfire. Activity of the positive control (100 nM CP-55,940) was set as 100% efficacy.

### 2. Human CB1 Radioligand Binding Assay

The CB1 binding assay was performed as described above except that HEK 293 EBNA cells expressing human cannabinoid receptor type 1 (Perkin Elmer cat #RBHCB1M) were used according the manufacturer's instructions. The incubation buffer was composed of 50 mM Tris-HCl pH 7.4, 2.5 mM EDTA, 5 mM MgCl₂, 5.0 mg/ml fatty acid free BSA and the wash buffer was composed of 50 mM Tris-HCl pH 7.4, 2.5 mM EDTA, 5 mM MgCl₂, 0.5 mg/ml fatty acid free BSA. Activity of the positive control (100 nM CP-55,940) was set as 100% efficacy.

### Membrane Preparation

Stable recombinant Human CB2-CHO cell membranes were prepared as follows: Chinese Hamster Ovarian (CHO) cells stably expressing human cannabinoid receptor type 2 were grown to ˜90% confluence in 15×100 mm culture dishes under puromycin selection (5 ug/ml) Cells were harvested from culture flasks using a cell scraper, were washed once with cold phosphate-buffered saline (calcium and magnesium free) and pelleted by centrifugation at 400 g for 5 min at 4° C. Cell pellet was washed once with cold phosphate-buffered saline and centrifuged again at 400×g for 5 min at 4° C. The pellet was suspended in ice-cold lysis buffer (10 mM Tris-HCl, 0.1 mM EDTA, containing 0.32 mM sucrose, pH 7.5) and homogenized in a chilled 7 ml glass dounce homogenizer using 50 strokes. The homogenate was centrifuged at 400 g for 15 min at 4° C. The cloudy supernatant was collected and centrifuged at 41000 g for 30 min at 4° C. The resulting pellet was washed with ice-cold sucrose-free lysis buffer and centrifuged again at 41000 g for 30 min at 4° C. The membrane pellet was suspended in sucrose-free storage buffer (10 mm Tris-HCl, 0.1 mM EDTA, pH 7.5) to a concentration of 2-3 mg protein/ml. Aliquots were flash frozen in liquid nitrogen and stored at −80° C. Concentration was determined using Dc protein assay kit (Bio-Rad).

TABLE 1
In Vitro Biological Activity Assays

| | CB2 Ligand | Selectivity, |
|---|---|---|
| | Binding Asssay | CB2 vs. CB1 |
| Example | + indicates EC50 < 1 uM | + indicates > 10-fold |
| No. | − indicates EC50 > 1 uM | − indicates < 10-fold |
| 1 | − | − |
| 2 | + | + |
| 3 | + | ND |
| 4 | NT | ND |
| 5 | − | + |
| 6 | − | ND |
| 7 | + | + |
| 8 | + | − |
| 9 | + | ND |
| 10 | − | − |
| 11 | − | − |
| 12 | − | − |
| 13 | − | − |
| 14 | NT | ND |
| 15 | + | ND |
| 16 | − | ND |
| 17 | NT | ND |
| 18 | + | + |
| 19 | − | − |
| 20 | NT | ND |
| 21 | − | − |
| 22 | NT | ND |
| 23 | + | + |
| 24 | + | − |
| 25 | + | + |
| 26 | NT | ND |
| 27 | + | + |
| 28 | + | + |
| 29 | − | − |
| 30 | NT | ND |
| 31 | + | + |
| 32 | − | + |
| 33 | + | − |
| 34 | + | + |
| 35 | + | − |
| 36 | + | + |
| 37 | + | + |
| 38 | NT | ND |
| 39 | NT | ND |
| 40 | NT | ND |
| 41 | − | − |
| 42 | NT | ND |
| 43 | NT | ND |
| 44 | NT | ND |
| 45 | + | − |
| 46 | − | − |
| 47 | + | − |
| 48 | − | − |
| 49 | + | − |
| 50 | + | − |
| 51 | − | − |
| 52 | − | − |
| 53 | − | − |
| 54 | − | − |
| 55 | NT | ND |
| 56 | − | − |
| 57 | − | − |
| 58 | − | − |
| 59 | + | + |
| 60 | + | − |
| 61 | NT | ND |
| 62 | + | − |
| 63 | + | − |
| 64 | + | − |
| 65 | + | + |
| 66 | + | + |
| 67 | NT | ND |
| 68 | − | ND |
| 69 | − | ND |
| 70 | − | ND |
| 71 | NT | ND |
| 72 | NT | ND |
| 73 | − | − |
| 74 | − | − |
| 75 | + | − |
| 76 | NT | ND |
| 77 | − | − |
| 78 | − | − |
| 79 | + | − |
| 80 | − | − |
| 81 | NT | ND |
| 82 | − | − |
| 83 | + | + |
| 84 | + | + |
| 85 | + | + |
| 86 | + | + |
| 87 | − | + |
| 88 | + | + |
| 89 | − | − |
| 90 | NT | ND |
| 91 | + | + |
| 92 | + | + |
| 93 | + | − |
| 94 | + | − |
| 95 | + | + |
| 96 | − | − |
| 97 | − | − |
| 98 | NT | ND |
| 99 | NT | ND |
| 100 | − | − |
| 101 | − | − |
| 102 | − | − |
| 103 | − | − |
| 104 | − | − |
| 105 | − | − |
| 106 | + | + |
| 107 | − | − |
| 108 | − | − |
| 109 | − | − |
| 110 | + | − |
| 111 | − | − |
| 112 | − | − |
| 113 | − | − |
| 114 | + | − |
| 115 | − | + |
| 116 | + | − |
| 117 | + | − |
| 118 | + | − |
| 119 | − | − |
| 120 | − | − |
| 121 | − | − |
| 122 | + | + |
| 123 | NT | ND |
| 124 | − | − |
| 125 | − | − |
| 126 | − | − |
| 127 | − | − |
| 128 | NT | ND |
| 129 | + | + |
| 130 | + | + |
| 131 | + | + |
| 132 | + | − |
| 133 | NT | ND |
| 134 | + | + |
| 135 | + | + |
| 136 | − | + |
| 137 | NT | ND |
| 138 | − | − |
| 139 | − | + |
| 140 | − | − |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I 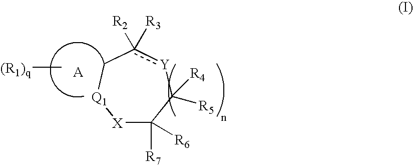
Or a salt, ester, or prodrug thereof, wherein:
A is a five- or six-membered monocyclic heterocycloalkyl or heteroaryl ring;
X is selected from the group consisting of CR₈R₉ and O;
Y is selected from the group consisting of NR₁₀ and CR₁₁R₁₂;
Q₁ is selected from the group consisting of N and CR₁₃;
n is an integer from 0 to 2;
q is an integer from 0 to 4;
each R₁ is independently selected from the group consisting of hydrogen, null, acyl, alkyl, alkenyl, alkynyl, alkoxy, amido, amino, aryl, aryloxy, carbamate, carboxy, cyano, cycloalkyl, halo, heteroalkyl, heteroaryl, heterocycloalkyl, hydroxyl, nitro, perhaloalkoxy, perhaloalkyl, and sulfonamide, any of which may be optionally substituted;
R₂ and R₃ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₄, R₅, R₆, R₇, R₈, and R₉ are each independently selected from the group consisting of hydrogen, null, alkyl, alkenyl, alkynyl, aryl, heteroaryl, cycloalkyl, and heterocycloalkyl, any of which may be optionally substituted; or R₆ and R₇ are taken together to form oxo (═O);
R₁₀ is selected from the group consisting of hydrogen, alkyl, alkenyl, alkynyl, aryl, arylalkyl, heteroaryl, cycloalkyl, heterocycloalkyl, —C(O)R₁₄, —C(O)NR₁₅R₁₆, and sulfonyl, any of which may be optionally substituted;
R₁₁ and R₁₂ are each independently selected from the group consisting of hydrogen, null, alkyl, alkenyl, alkynyl, aryl, arylalkyl, heteroaryl, cycloalkyl, heterocycloalkyl, —C(O)NR₁₅R₁₆, —NR₁₇C(O)NR₁₈R₁₉, —NR₂₀C(O)OR₂₁, and sulfonyl, any of which may be optionally substituted;
R₁₃, R₁₅, R₁₇, R₁₉ and R₂₀ are each independently selected from the group consisting of hydrogen, null, and lower alkyl; and
R₁₄, R₁₆, R₁₈ and R₂₁ are each independently selected from the group consisting of hydrogen, alkyl, alkenyl, alkynyl, aryl, heteroaryl, cycloalkyl, heterocycloalkyl, arylalkyl, heteroarylalkyl, and cycloalkylalkyl, any of which may be optionally substituted.

2. The compound as recited in claim 1, wherein said compound has structural Formula II 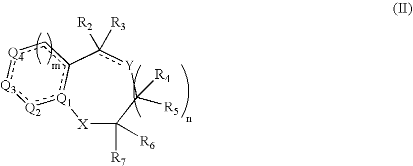
Or a salt, ester, or prodrug thereof, wherein:
X is selected from the group consisting of CR₈R₉ and O;
Y is selected from the group consisting of NR₁₀ and CR₁₁R₁₂;
Q₁ is selected from the group consisting of N and CR₁₃;
Q₂ is selected from the group consisting of N, NR₂₂, CR₂₃, and CR₂₃R₂₄;
Q₃ is selected from the group consisting of N, NR₂₅, CR₂₆, CR₂₆R₂₇, S, and O;
Q₄ is selected from the group consisting of N, NR₂₈, CR₂₉, CR₂₉R₃₀, S, and O;
n is an integer from 0 to 2;
m is an integer from 0 to 2;
R₂ and R₃ are independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₄, R₅, R₆, R₇, R₈, and R₉ are each independently selected from the group consisting of hydrogen, null, alkyl, alkenyl, alkynyl, aryl, heteroaryl, cycloalkyl, and heterocycloalkyl, any of which may be optionally substituted; or R₆ and R₇ are taken together to form oxo (═O);
R₁₀ is selected from the group consisting of hydrogen, alkyl, alkenyl, alkynyl, aryl, arylalkyl, heteroaryl, cycloalkyl, heterocycloalkyl, —C(O)R₁₄, —C(O)NR₁₅R₁₆, and sulfonyl, any of which may be optionally substituted;
R₁₁ and R₁₂ are each independently selected from the group consisting of hydrogen, null, alkyl, alkenyl, alkynyl, aryl, arylalkyl, heteroaryl, cycloalkyl, heterocycloalkyl, —C(O)NR₁₅R₁₆, —NR₁₇C(O)NR₁₈R₁₉, —NR₂₀C(O)OR₂₁, and sulfonyl, any of which may be optionally substituted;
R₁₃, R₁₅, R₁₇, R₁₉, R₂₀, R₂₂, R₂₅, and R₂₈ are each independently selected from the group consisting of hydrogen, null, and lower alkyl; and
R₁₄, R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

3. The compound as recited in claim 2, wherein:
Y is NR₁₀; and
n is 1.

4. The compound as recited in claim 3, wherein:
R₂, R₄, R₅, R₆, and R₇ are hydrogen; and
R₁₀ is —C(O)NR₁₅R₁₆.

5. The compound as recited in claim 4, wherein R₁₅ is hydrogen.

6. The compound as recited in claim 5, wherein said compound has structural Formula III 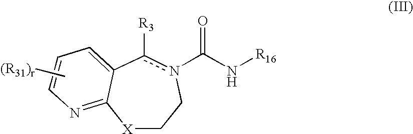
Or a salt, ester, or prodrug thereof, wherein:
X is selected from the group consisting of CR₈R₉ and O;
r is an integer from 0 to 3;
R₃ is selected from the group consisting of hydrogen alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₈ and R₉ are each independently selected from the group consisting of hydrogen, null, alkyl, alkenyl, alkynyl, aryl, heteroaryl, cycloalkyl, and heterocycloalkyl, any of which may be optionally substituted;
R₁₆ is selected from the group consisting of aryl, heteroaryl, and arylalkyl, any of which may be optionally substituted; and
each R₃₁ is independently selected from the group consisting of hydrogen, null, acyl, alkyl, alkenyl, alkynyl, alkoxy, amido, amino, aryl, aryloxy, carbamate, carboxy, cyano, cycloalkyl, halo, heteroalkyl, heteroaryl, heterocycloalkyl, hydroxyl, nitro, perhaloalkoxy, perhaloalkyl, and sulfonamide, any of which may be optionally substituted.

7. The compound as recited in claim 6, wherein R₃ is aryl, which may be optionally substituted with one or more substituents selected from the group consisting of hydrogen, lower alkyl, and halo.

8. The compound as recited in claim 7, wherein X is O.

9. The compound as recited in claim 7, wherein:
X is CR₈R₉; and
R₈ and R₉ are each independently hydrogen.

10. The compound as recited in claim 2, wherein m is 0.

11. The compound as recited in claim 10, wherein:
Q₁ is N; and
R₂, R₄, R₅, R₆, and R₇ are each independently hydrogen.

12. The compound as recited in claim 11, wherein:
X is CR₈R₉; and
R₈ and R₉ are hydrogen.

13. The compound as recited in claim 12, wherein R₃ is selected from the group consisting of aryl, cycloalkyl, and arylalkyl, any of which may be optionally substituted.

14. The compound as recited in claim 13, wherein said compound has structural Formula IV 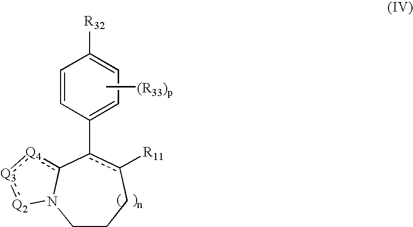
Or a salt, ester, or prodrug thereof, wherein:
Q₂ is selected from the group consisting of N, NR₂₂, CR₂₃, and CR₂₃R₂₄;
Q₃ is selected from the group consisting of N, NR₂₅, CR₂₆, CR₂₆R₂₇, S, and O;
Q₄ is selected from the group consisting of N, NR₂₈, CR₂₉, and CR₂₉R₃₀;
n is an integer from 0 to 2;
p is an integer from 0 to 4;
R₁₁ is selected from the group consisting of —C(O)NR₁₅R₁₆, —NR₁₇C(O)NR₁₈R₁₉, and —NR₂₀C(O)OR₂₁;
R₁₅, R₁₇, R₁₉, R₂₀, R₂₂, R₂₅, and R₂₈ are each independently selected from the group consisting of hydrogen, null, and lower alkyl;
R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and
each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

15. The compound as recited in claim 14, wherein:
Q₂ is selected from the group consisting of N and CR₂₃;
Q₃ is selected from the group consisting of N and CR₂₆;
Q₄ is selected from the group consisting of N and CR₂₉;
the optional double bonds between Q₂ and Q₃, and between Q₄ and the adjacent carbon, are each present;
the optional double bond between Q₃ and Q₄ is absent; and
R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

16. The compound as recited in claim 15, wherein:
n is an integer from 0 to 1; and
p is 0.

17. The compound as recited in claim 16, wherein:
R₁₅, R₁₇, and R₁₉, and R₂₀ are each independently hydrogen; and
R₁₆, R₁₈, R₂₀, and R₂₁ are each independently selected from the group consisting of aryl and arylalkyl, any of which may be optionally substituted with a substituent selected from the group consisting of hydrogen, alkoxy, lower alkyl, halo, perhaloalkoxy, and perhaloalkyl.

18. The compound as recited in claim 17, wherein:
Q₂ is CR₂₃;
Q₃ is CR₂₆;
Q₄ is CR₂₉;
R₂₃, R₂₆, and R₂₉ are each independently hydrogen; and
R₃₂ is selected from the group consisting of hydrogen, lower alkyl, alkoxy, cyanoalkyl, and haloalkyl.

19. The compound as recited in claim 17, wherein:
Q₂ is CR₂₃;
Q₃ is CR₂₆;
Q₄ is N;
R₂₃ and R₂₆ are each independently hydrogen; and
R₃₂ is selected from the group consisting of hydrogen, lower alkyl, alkoxy, cyanoalkyl, and haloalkyl.

20. The compound as recited in claim 17, wherein:
Q₂ is CR₂₃;
Q₃ is N;
Q₄ is CR₂₉;
R₂₃ and R₂₉ are each independently hydrogen; and
R₃₂ is selected from the group consisting of hydrogen, lower alkyl, alkoxy, cyanoalkyl, and haloalkyl.

21. The compound as recited in claim 17, wherein:
Q₂ is N;
Q₃ is CR₂₃;
Q₄ is CR₂₄;
R₂₃ and R₂₄ are each independently hydrogen; and
R₂₅ is selected from the group consisting of hydrogen, lower alkyl, alkoxy, cyanoalkyl, and haloalkyl.

22. The compound as recited in any one of claims 18-21, wherein n is 0.

23. The compound as recited in any one of claims 18-21, wherein n is 1.

24. A compound, as recited in claim 14, having structural formula V 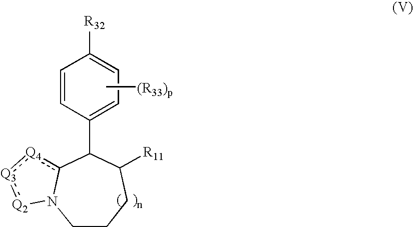
Or a salt, ester, or prodrug thereof, wherein:
Q₂ is selected from the group consisting of N, NR₂₂, CR₂₃, and CR₂₃R₂₄;
Q₃ is selected from the group consisting of N, NR₂₅, CR₂₆, CR₂₆R₂₇, S, and O;
Q₄ is selected from the group consisting of N, NR₂₈, CR₂₉, and CR₂₉R₃₀;
n is an integer from 0 to 2;
p is an integer from 0 to 4;
R₁₁ is selected from the group consisting of —C(O)NR₁₅R₁₆, —NR₁₇C(O)NR₁₈R₁₉, and, —NR₂₀C(O)OR₂₁;
R₁₅, R₁₇, R₁₉, R₂₀, R₂₂, R₂₅, and R₂₈ are each independently selected from the group consisting of hydrogen, null, and lower alkyl;
R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and
each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

25. The compound as recited in claim 24, wherein:
Q₂ is selected from the group consisting of N, and CR₂₃;
Q₃ is selected from the group consisting of N and CR₂₆;
Q₄ is selected from the group consisting of N and CR₂₉;
the optional double bonds between Q₂ and Q₃ and Q₄ and the adjacent carbon are each present;
the optional double bond between Q₃ and Q₄ is absent; and
R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

26. The compound as recited in claim 25, wherein:
n is an integer from 0 to 1; and
p is 0.

27. The compound as recited in claim 26, wherein:
R₁₅, R₁₇, and R₁₉ are each independently hydrogen; and
R₁₆, R₁₈, R₂₀, and R₂₁ are each independently selected from the group consisting of aryl and arylalkyl, any of which may be optionally substituted with a substituent selected from the group consisting of hydrogen, alkoxy, lower alkyl, halo, perhaloalkoxy, and perhaloalkyl.

28. The compound as recited in claim 27, wherein:
Q₂ is CR₂₃;
Q₃ is CR₂₆;
Q₄ is CR₂₉;
R₂₃, R₂₆, and R₂₉ are each independently hydrogen; and
R₃₂ is selected from the group consisting of hydrogen, lower alkyl, alkoxy, cyanoalkyl, and haloalkyl.

29. The compound as recited in claim 27, wherein:
Q₂ is CR₂₃;
Q₃ is CR₂₆;
Q₄ is N;
R₂₃ and R₂₆ are each independently hydrogen; and
R₃₂ is selected from the group consisting of hydrogen, lower alkyl, alkoxy, cyanoalkyl, and haloalkyl.

30. The compound as recited in claim 27, wherein:
Q₂ is CR₂₃;
Q₃ is N;
Q₄ is CR₂₉;
R₂₃ and R₂₉ are each independently hydrogen; and
R₃₂ is selected from the group consisting of hydrogen, lower alkyl, alkoxy, cyanoalkyl, and haloalkyl.

31. The compound as recited in claim 27, wherein:
Q₂ is N;
Q₃ is CR₂₃;
Q₄ is CR₂₄;
R₂₃ and R₂₄ are each independently hydrogen; and
R₂₅ is selected from the group consisting of hydrogen, lower alkyl, alkoxy, cyanoalkyl, and haloalkyl.

32. The compound as recited in any one of claims 28-31, wherein n is 0.

33. The compound as recited in any one of claims 28-31, wherein n is 1.

34. The compound as recited in claim 13, wherein:
Y is NR₁₀; and
R₁₀ is selected from the group consisting of aryl, arylalkyl, heteroaryl, cycloalkyl, heterocycloalkyl, —C(O)R₁₄, —C(O)NR₁₅R₁₆, any of which may be optionally substituted.

35. The compound as recited in claim 34, wherein R₁₀ is selected from the group consisting of aryl, —C(O)R₁₄, —C(O)NR₁₅R₁₆, any of which may be optionally substituted.

36. The compound as recited in claim 35, wherein said compound has structural Formula VI 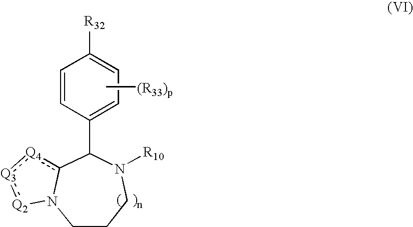
Or a salt, ester, or prodrug thereof, wherein:
Q₂ is selected from the group consisting of N, NR₂₂, CR₂₃, and CR₂₃R₂₄;
Q₃ is selected from the group consisting of N, NR₂₅, CR₂₆, CR₂₆R₂₇, S, and O;
Q₄ is selected from the group consisting of N, NR₂₈, CR₂₉, and CR₂₉R₃₀;
n is an integer from 0 to 2;
p is an integer from 0 to 4;
R₁₀ is selected from the group consisting of —C(O)R₁₄, —C(O)NR₁₅R₁₆, and aryl, which may be optionally substituted;
R₁₅, R₁₇, R₁₉, R₂₀, R₂₂, R₂₅, and R₂₈ are each independently selected from the group consisting of hydrogen, null, and lower alkyl;
R₁₄, R₁₆, R₁₈, R₂₁, R₂₃, R₂₄, R₂₆, R₂₇, R₂₉, and R₃₀ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted;
R₃₂ is independently selected from the group consisting of hydrogen, null, acyl, alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and
each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

37. The compound as recited in claim 36, wherein:
Q₂ is selected from the group consisting of N and CR₂₃;
Q₃ is selected from the group consisting of N and CR₂₆;
Q₄ is selected from the group consisting of N and CR₂₉;
the optional double bonds between Q₂ and Q₃ and Q₄ and the adjacent carbon are each present;
the optional double bond between Q₃ and Q₄ is absent; and
R₂₃, R₂₆, and R₂₉ are each independently selected from the group consisting of hydrogen, null, alkyl, alkenyl, alkynyl, aryl, heteroaryl, cycloalkyl, heterocycloalkyl, arylalkyl, heteroarylalkyl, and cycloalkylalkyl, any of which may be optionally substituted.

38. The compound as recited in claim 37, wherein:
n is an integer from 0 to 1;
p is 0;
R₁₄ and R₁₆ are each independently selected from the group consisting of aryl, arylalkyl, heteroaryl, any of which may be optionally substituted; and
R₁₅ is hydrogen;
R₃₂ is independently selected from the group consisting of hydrogen, lower alkyl, alkoxy,cyanoalkyl, halo, haloalkyl, heteroalkyl, heteroaryl, heterocycloalkyl, hydroxyl, nitro, perhaloalkoxy, perhaloalkyl, and sulfonamide, any of which may be optionally substituted; and
each R₃₃ are each independently selected from the group consisting of hydrogen, null, acyl, C₂-C₆ alkyl, alkenyl, alkynyl, alkoxy, amido, amino, aryl, aryloxy, carbamate, carboxy, cyano, cyanoalkyl, cycloalkyl, halo, haloalkyl, heteroalkyl, heteroaryl, heterocycloalkyl, hydroxyl, nitro, perhaloalkoxy, perhaloalkyl, and sulfonamide, any of which may be optionally substituted.

39. The compound as recited in claim 38, wherein n is 1.

40. The compound as recited in claim 39, wherein R₁₀ is —C(O)R₁₄.

41. The compound as recited in claim 40, wherein:
Q₂ is CR₂₃;
Q₃ is CR₂₆;
Q₄ is CR₂₉;
R₂₃, R₂₆, and R₂₉ are each independently hydrogen; and
R₃₂ is selected from the group consisting of hydrogen, lower alkyl, alkoxy, cyanoalkyl, and haloalkyl.

42. The compound as recited in claim 41, wherein:
Q₂ is CR₂₃;
Q₃ is CR₂₆;
Q₄ is N;
R₂₃ and R₂₆ are each independently hydrogen; and
R₃₂ is selected from the group consisting of hydrogen, lower alkyl, alkoxy, cyanoalkyl, and haloalkyl.

43. The compound as recited in claim 41, wherein:
Q₂ is CR₂₃;
Q₃ is N;
Q₄ is CR₂₉;
R₂₃ and R₂₉ are each independently hydrogen; and
R₃₂ is selected from the group consisting of hydrogen, lower alkyl, alkoxy, cyanoalkyl, and haloalkyl.

44. The compound as recited in claim 41, wherein:
Q₂ is N;
Q₃ is CR₂₃;
Q₄ is CR₂₄;
R₂₃ and R₂₄ are each independently hydrogen; and
R₂₅ is selected from the group consisting of hydrogen, lower alkyl, alkoxy, cyanoalkyl, and haloalkyl.

45. The compound as recited in claim 2, wherein R₂, R₄, R₅, R₆, and R₇ are hydrogen.

46. The compound as recited in claim 45, wherein:
X is CR₈R₉; and
R₈ and R₉ are each independently hydrogen.

47. The compound as recited in claim 46, wherein:
Y is NR₁₀; and
R₃ is selected from the group consisting of aryl and arylalkyl, any of which may be optionally substituted.

48. The compound as recited in claim 47, wherein Q₁ is N.

49. The compound as recited in claim 48, wherein R₃ is aryl, which may be optionally substituted in the para-position with a substituent selected from the group consisting of hydrogen, lower alkyl, alkoxy, cyanoalkyl, and haloalkyl.

50. The compound as recited in claim 49, wherein:
R₁₀ is selected from the group consisting of —C(O)R₁₄ and —C(O)NR₁₅R₁₆;
R₁₄ and R₁₆ are each independently selected from the group consisting of lower alkyl, aryl, and arylalkyl, any of which may be optionally substituted; and
R₁₅ is hydrogen.

51. The compound as recited in claim 50, wherein:
n is an integer from 0 to 1;
m is 0; and
the optional double bonds between Q₁ and Q₂, Q₂ and Q₃, and Q₃ and Q₄ are each absent.

52. The compound as recited in claim 51, wherein:
Q₂ is CR₂₃R₂₄;
Q₃ is selected from the group consisting of NR₂₂, CR₂₆R₂₇, S, and O;
Q₄ is CR₂₉R₃₀;
R₂₂ is selected from the group consisting of hydrogen and lower alkyl; and
R₂₃, R₂₄, R₂₆, R₂₇, R₂₉, and R₃₀ are each independently selected from the group consisting of hydrogen, lower alkyl, alkenyl, and alkynyl, any of which may be optionally substituted.

53. The compound as recited in claim 52, wherein:
Q₃ is NR₂₂; and
R₂₂ is selected from the group consisting of hydrogen and lower alkyl.

54. The compound as recited in claim 53, wherein n is 0.

55. The compound as recited in claim 53, wherein n is 1.

56. A compound selected from the group consisting of Examples 1 to 140.

57. A compound as recited in claim 1 for use as a medicament.

58. A compound as recited in claim 1 for use in the manufacture of a medicament for the prevention or treatment of a disease or condition ameliorated by the modulation of CB2.

59. A pharmaceutical composition comprising a compound as recited in claim 1 together with a pharmaceutically acceptable carrier.

60. A method of modulation of CB2 comprising contacting CB2 with a compound as recited in claim 1.

61. A method of treatment of a CB2-mediated disease comprising the administration of a therapeutically effective amount of a compound as recited in claim 1 to a patient in need thereof.

62. The method as recited in claim 61 wherein said disease is selected from the group consisting of acute nociceptive pain, chronic nociceptive pain, neuropathic pain, inflammatory pain, abdominal pain, acute herpes zoster, postherpetic neuralgia, fibromyalgia, ocular pain, muscle spasm, neuromuscular disorder, atherosclerosis progression, tactile allodynia, hyperalgesia, post-surgical pain, bone fracture pain, dental pain, bunionectomy, muscular pain, mastalgia, pain from dermal injuries, lower back pain, headaches, migraine, osteoarthritis, musculoskeletal conditions, cancer pain, reflex sympathetic dystrophy/causalgia, peripheral neuropathy, diabetic neuropathy, complex regional pain syndrome, entrapment neuropathy, multiple sclerosis, rheumatoid arthritis, systemic lupus erythematosus, myasthenia gravis, autoimmune disease, malabsorption syndrome, pulmonary disease, osteoporosis, atherosclerosis, diabetes mellitus type I, inflammatory bowel disease, irritable bowel syndrome, psoriasis, tissue rejection in organ transplants, celiac disease, asthma, glaucoma, Sjogren's syndrome, chronic liver disease, acute liver disease, liver fibrosis, ischemia-reperfusion injury, hepatic encephalopathy and non-alcoholic fatty liver disease (NAFLD).

63. A method of treatment of a CB2-mediated disease comprising the administration of:
a. a therapeutically effective amount of a compound as recited in claim 1; and
b. another therapeutic agent.

64. A method for achieving an effect in a patient comprising the administration of a therapeutically effective amount of a compound as recited in claim 1 to a patient, wherein the effect is selected from the group consisting of anti-emesis, enhancement of appetite, vascular hypotension, immunomudulation, analgesia, treatment of muscle spasm, treatment of neuromuscular disorders, treatment of osteoporosis, and treatment of atherosclerosis.
